(19) United States
(12) Patent Application Publication
(10) Pub. No.: US 2023/0077104 A1
(43) Pub. Date: Mar. 9, 2023

(54) L-SEPIAPTERIN AND METHODS OF USE FOR TREATING DISEASES AND DISORDERS

(71) Applicants: THE UNITED STATES GOVERNMENT AS REPRESENTED BY THE DEPARTMENT OF VETERAN AFFAIRS, Washington, DC (US); MUSC FOUNDATION FOR RESEARCH DEVELOPMENT D/B/A ZUCKER INSTITUTE FOR INNOVATION, Charleston, SC (US)

(72) Inventors: James Oates, Charleston, SC (US); Joy Jones Buie, Charleston, SC (US)

(21) Appl. No.: 17/800,207

(22) PCT Filed: Feb. 19, 2021
(86) PCT No.: PCT/US21/18723

§ 371 Date: Aug. 16, 2022

Related U.S. Application Data
(60) Provisional application No. 62/978,468, filed on Feb. 19, 2020.
(60) Provisional application No. 62/994,386, filed on Mar. 25, 2020.

(51) Int. Cl. A61K 31/519; A61K 31/04; A61K 31/345; A61K 31/275; A61P 37/06

(52) U.S. Cl. CPC A61K 31/519; A61K 31/04; A61K 31/345; A61K 31/275; A61P 37/06

(57) ABSTRACT

The present invention provides compositions and methods for increasing nitric oxide production for treating or preventing a chronic inflammatory autoimmune disease or disorder.

### CROSS REFERENCE TO RELATED APPLICATIONS

This application claims priority to U.S. Provisional Application No. 62/978,468 filed Feb. 19, 2020 and to U.S. Provisional Application No. 62/994,386, filed Mar. 25, 2020, each of which is hereby incorporated by reference herein in its entirety.

### STATEMENT REGARDING FEDERALLY SPONSORED RESEARCH OR DEVELOPMENT

This invention was made with government support under Grant Nos. AR072583 and AR0451476 awarded by the National Institutes of Health and Grant No. CX001248 awarded by the Department of Veterans Affairs. The government has certain rights in the invention.

### BACKGROUND OF THE INVENTION

Systemic lupus erythematosus (SLE) is a heterogeneous chronic autoimmune inflammatory syndrome that predominately impacts women of childbearing age. Although specific clinical and immunological criteria have been defined, its clinical course is highly variable with some patients experiencing life-threatening cardiovascular disease complications which account for one-third of deaths in patients with SLE (Lerang, K. et al., 2014, Lupus, 23:1546-52; Björnådal L. et al., 2004, J. Rheumatol., 31:713-9). A significant proportion of patients with SLE display accelerated endothelial dysfunction which precedes cardiovascular disease (CVD). The endothelium plays a pivotal role in governing vascular function and, thus, prevents the development of vascular abnormalities (van Paassen, P. et al., 2007, Ann. NY Acad. Sci., 1108:147-56; Bruce, I. N. et al., 2003, J. Rhematol., 30:288-91; Bruce, I. N. et al., 2000, J. Rheumatol., 27:2378-7). Chronic inflammation promotes endothelial cell injury leading to generation of superoxide and expression of cell adhesion markers (Castellon, X. et al., 2016, Aging Dis., 7:81-9; Gremmel, T. et al., 2015, PLoS One, 10:e122586). These perturbations in endothelial cell function promote oedema, leucocyte trafficking and organ damage. While causes of endothelial dysfunction in SLE are multifactorial, the specific biological underpinnings governing the development of endothelial dysfunction in SLE are incompletely understood and represent an important area of research.

Nitric oxide (NO) is a membrane-permeable free radical, continuously synthesized by the endothelial nitric oxide synthase (eNOS) dimeric enzyme (Alderton, W. K. et al., 2001. Biochem. J., 357:593-615). Diffusion of NO across the cellular membrane as a paracrine mediator has impacts on cellular function critical for blood vessel dilation and unobstructed blood flow (Miersch, S. et al., 2008, J. Biol. Chem., 283:18513-21; Forstermann U., 1986, J Cardiovasc Pharmacol. 8 (Suppl 10):S45-51; Ignarro, L. J. et al., 1986, J. Pharmacol. Exp. Ther., 237:893-900). Diminished release of NO and loss of eNOS expression have been consistently linked to endothelial dysfunction (Napoli, C. et al., 2001, Nitric Oxide, 5:88-97; Napoli, C. et al., 2006, Nitric Oxide, 15:265-79). Paradoxically, uncoupled eNOS, likely resulting from oxidation of tetrahydrobiopterin (BH₄), leads to eNOS enzymatic dysfunction (Shinozaki, K. et al., 1999, Diabetes, 484:2437-45; Hong, H. J. et al., 2001, Hypertension, 38:1044-8; Schumacher, M. et al., 1997, J. Am. Coll. Cardiol., 30:703-7). Biopsies from patients with severe lupus nephritis show diminished eNOS expression (Furusu, A. et al., 1998, Kidney Int., 53:1760-8; Bollain-y-Goytia J. J. et al., 2009, Inflamm. Res., 58:61-6). Accordingly, the previous works demonstrated that genetic ablation of eNOS in lupus-prone MRL/lpr mice resulted in accelerated, more severe disease with significant declines in survival (Gilkeson, G. S. et al., 2013, PLoS One, 8:e64650). MRL/lpr mice lacking eNOS display increased superoxide production associated with increased MCP1 production, increased glomerular crescentic and necrotic lesions, and reduced levels of the anti-inflammatory cytokine interleukin (IL)-10 (Al Gadban, M. M. et al., 2012, Cell Immunol., 276:42-51). Accordingly, a positive correlation between H₂O₂ and eNOS levels in vitro exists (Drummond, G. R. et al., 2000, Circ. Res., 86:347-54). NO produced in the presence of O₂.⁻ yields peroxynitrite (ONOO⁻) which oxidises the essential eNOS co-factor BH₄ to dihydrobiopterin (BH₂) and biopterin. This oxidation of BH₄ leads to uncoupling of the eNOS homodimer to monomers, resulting in reactive oxygen species (ROS) rather than NO production (Karbach, S. et al., 2014, Curr. Pharm. Des., 20:3579-94; Szelenyi, Z. et al., 2015, J. Geriatr. Cardiol., 12:1-10). ROS production itself leads to endothelial dysfunction. L-sepiapterin (L-sep) is a precursor for tetrahydrobiopterin (BH₄) synthesis, and previous studies have shown its efficacy in restoring eNOS function, possibly through a recoupling mechanism (Mata-Greenwood, E. et al., 2006, Am. J. Physiol. Lung Cell Mol. Physiol., 290:L232-L241). Thus, L-sep may serve as a viable therapeutic option when eNOS uncoupling is the predominant mechanism of endothelial dysfunction in an inflammatory microenvironment, such is present in patients with SLE.

A hallmark of endothelial inflammatory responses which coincide with diminished endothelial-NO is increased neutrophil migration and adhesion to the endothelial cell surface (Dal Secco, D. et al., 2003, Nitric Oxide, 9:153-64; Hossain, M. et al., 2012, J. Inflamm., 9:28). Neutrophil migration and adhesion are processes that promote progression of atherosclerosis through the release of myeloperoxidase, promoting ROS production, and sequestering monocytes to the intravascular space (Odobasic, D. et al., 2016, J. Immunol. Res., 2016:2349817:1-17). Recent studies link NETosis, a process of neutrophil death emerging as a potential pathogenic process in SLE, to atherosclerosis (Mozzini, C. et al., 2017, Intern. Emerg. Med., 12:13-22).

There remains a need in the art for compositions and methods for treating chronic inflammatory autoimmune diseases and disorders. The present invention satisfies this need.

### SUMMARY OF THE INVENTION

In one embodiment, the invention relates to a composition for treating or preventing a chronic inflammatory autoimmune disease or disorder comprising a stimulator of nitric oxide (NO) production.

In one embodiment, the stimulator of NO production is a small molecule chemical compound. In one embodiment, the small molecule chemical compound is 2-amino-6-[-2-hydroxypropanoyl]-7,8-dihydro-1H-pteridin-4-one (L-Sepiapterin), tetrahydrobiopterin (BH4), nitroglycerin (GTN), isosorbide dinitrate (ISDN), isosorbide mononitrate (IS-5N), amyl nitrite, sodium nitroprusside (SNP), pentaerythrityltetranitrate (PETN), nicorandil or molsidomine a derivative thereof, and a salt thereof.

In one embodiment, the composition further comprises an immunosuppressive agent.

In one embodiment, the disease or disorder is systemic lupus erythematosus (SLE), lupus nephritis, autoimmunity-related cardiovascular disease (CVD) and atherosclerosis, hypertension, systemic sclerosis, celiac disease, inflammatory bowel disease, type 1 diabetes, glomerulonephritis, Crohn's disease, psoriatic arthritis, or rheumatoid arthritis.

In one embodiment, the invention relates to a method for treating or preventing a chronic inflammatory autoimmune disease or disorder comprising administrating a composition comprising a stimulator of nitric oxide (NO) production to a subject in need thereof.

In one embodiment, the stimulator of NO production is a small molecule chemical compound. In one embodiment, the small molecule chemical compound is 2-amino-6-[-2-hydroxypropanoyl]-7,8-dihydro-1H-pteridin-4-one (L-Sepiapterin), tetrahydrobiopterin (BH4), nitroglycerin (GTN), isosorbide dinitrate (ISDN), isosorbide mononitrate (IS-5N), amyl nitrite, sodium nitroprusside (SNP), pentaerythrityltetranitrate (PETN), nicorandil or molsidomine a derivative thereof, and a salt thereof.

In one embodiment, the method further comprises administering an immunosuppressive agent.

In one embodiment, the disease or disorder is systemic lupus erythematosus (SLE), lupus nephritis, autoimmunity-related cardiovascular disease (CVD) and atherosclerosis, hypertension, systemic sclerosis, multiple sclerosis, celiac disease, inflammatory bowel disease, type 1 diabetes, glomerulonephritis, Crohn's disease, psoriatic arthritis, or rheumatoid arthritis.

In one embodiment, the disease or disorder is active.

In one embodiment, the method comprises administering the stimulator of NO production to a subject having inactive disease, whereby the method prevents the disease from becoming active.

In one embodiment, the stimulator of NO production is L-Sepiapterin.

In one embodiment, the L-Sepiapterin is administered at a dosage of between 5 and 50 mg/kg daily. In one embodiment, the L-Sepiapterin is administered at a dosage of 20 mg/kg daily.

In one embodiment, the subject is a human.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A through FIG. 1D depict the results of experiments demonstrating SLE sera-induced endothelial nitric oxide synthase (eNOS) mRNA expression in human umbilical vein endothelial cells. (FIG. 1A) eNOS mRNA levels from human umbilical vein endothelial cells (HUVECs) treated with buffer 20% control or SLE sera were as follows: for buffer controls (9), 1±0.06918; for healthy controls (14), 1.032±0.3294; for SLE (22) 1.888±0.2229 (mean±SEM). (FIG. 1B) Lupus-induced eNOS mRNA levels correlate with patient age but not control derived eNOS mRNA, p<0.01. (FIG. 1C) Lack of association between eNOS mRNA and SLE Disease Activity Index (SLEDAI) scores. (FIG. 1D) Lack of association between NOS3 expression and systolic blood pressure (BP) or diastolic BP measured in millimeter of mercury. *p<0.05, **p<0.001, ***p<0.0001. Samples were analyzed using a Kruskal-Wallis non-parametric multiple comparisons test and a Dunn's post-test and Spearman's correlation. r=rho value.

FIG. 2A through FIG. 2C depict the results of experiments demonstrating that L-sepiapterin (L-sep) restores nitric oxide (NO) production in SLE sera cultured humanumbilical vein endothelial cells (HUVECs). (FIG. 2A) Representative histograms of DAF-FM fluorescence intensity measured in HUVECs cultured in control conditions (grey) or control conditions+L-sepiapterin (black) in the following order endothelial basal medium 2 (EBM-2) buffer (far left), healthy control serum (middle) and SLE serum (far right). (FIG. 2B) A column graph representing the mean fold change of the median fluorescence intensity normalized to buffer controls±SEM. Analysis was conducted using a two-way analysis of variance (ANOVA) with Fisher's least significance difference post-hoc test. *p<0.05, **p<0.01. (FIG. 2C) A graph representing the mean fold change of L-sep/no L-sep for each serum treatment group. Analysis was conducted using a one-way ANOVA. ***p<0.001, compared with buffer control, ##p<0.01 compared with healthy control.

FIG. 3A and FIG. 3B depict schematics illustrating the postulated impact of SLE on endothelial nitric oxide (NO) synthase activity and endothelial nitric oxide synthase (eNOS) uncoupling. (FIG. 3A) In normal conditions, basal eNOS oxidises L-arginine to L-citrulline and produces NO. However, in the presence of components found in lupus serum, NAPDH oxidase is activated leading to reactive oxygen species and subsequent production of peroxynitrite. (FIG. 3B) Supplementation of low-dose L-sepiapterin in cell cultures raises intracellular BH4 levels leading to improvements in NO production in lupus serum cultured cells.

FIG. 4A and FIG. 4B depict the results of experiments demonstrating that serum does not cause mitochondrial dysfunction. (FIG. 4A) The cellular mitochondrial profile of human umbilical vein endothelial cells in different cell culture conditions defined by the use of the inhibitors, oligomycin (oligo), FCCP and antimycin A (AntiA). (FIG. 4B) The bioenergetic health index of endothelial cells in buffer (2% fetal bovine serum), healthy control serum (50% v/v), or SLE serum (50% v/v), calculated based on the cellular mitochondrial profile. Analysis was conducted using a one-way analysis of variance and Fisher's least significance difference post-test. BHI, bioenergetic health index; FCCP, trifluoromethoxy carbonylcyanide phenylhydrazone; OCR, oxygen consumption rate.

FIG. 5A and FIG. 5B depict the results of experiments demonstrating that lupus serum induces neutrophil chemotaxis. (FIG. 5A) Humanumbilical vein endothelial cells (HUVECs) were stimulated for 6 hours with 50% serum and transwell inserts containing 50 000 calcein AM neutrophils/insert were placed in the well as outlined in the ‘Materials and methods’ section. Images represent calcein am (green) stained neutrophils (first column), bright field+calcein AM stained (second column) of HUVEC cells exposed to endothelial basal medium 2 (EBM-2) (control), Interleukin (IL)-8 (positive control) healthy serum, or SLE serum (FIG. 5B) The graph represents the mean ratio of endothelial cells/neutrophils±SEM n=5 SLE, n=5 SLE+LN, n=5 healthy, n=3 buffer controls. All data were analyzed using Kruskal-Wallis analysis of variance and Dunn's post-test.++p<0.01 compared with healthy control.

FIG. 6A and FIG. 6B depict the results of experiments demonstrating that lupus serum induces neutrophil adhesion to the endothelial cell surface. (FIG. 6A) Human umbilical vein endothelial cells (HUVECs) were stimulated for 6 hours with 10% serum for 6 hours and stimulated cells were exposed to neutrophils isolated from fresh human blood as described elsewhere herein. Images are representative of 6/8 SLE sera and 4/5 control samples. Images represent calcein AM (green) stained neutrophils (first column), bright field+calcein AM stained (second column) of HUVEC cells exposed to endothelial basal medium 2 (EBM-2) (control), healthy serum, or SLE serum in the presence or absence of L-sepiapterin, 40× magnification. (FIG. 6B) Graph represents fluorescence intensity±SEM. Quantification of neutrophils are reported as means±SEM n=9 SLE, five controls. *p<0.05 compared with EBM-2, ++p<0.01 and +++p<0.001 compared with EBM-2+L-sepiapterin. All data were analyzed using two-way analysis of variance and Fisher's least significant difference post-test.

FIG. 7 depicts the Janus faces of endothelial nitric oxide synthase (eNOS). With adequate BH4 stores, eNOS homodimers are coupled. Electrons can progress from the reductase to the oxygenase domain, and NO is produced. NO modulates the activation of transcription factors such as NFκB. On the other hand, when superoxide (O2-.) from NADPH oxidase or uncoupled eNOS combines with NO to form ONOO—, this oxidizes BH4 to BH2, eNOS becomes uncoupled. In this conformation, electrons do not progress normally, and O2-. is synthesized. O2-. is reduced to hydrogen peroxide (H2O2), which activates transcription factors such as NFκB. Sepiapterin, an actively transported precursor to BH4 through the salvage pathway (sepiapterin reductase (SR) and dihydrofolate reductase (DHFR)) can restore coupling of eNOS by restoring BH4 levels.

FIG. 8 depicts the pathways of activation of NADPH oxidase in endothelial cells leading to inflammatory phenotype. Extracellular factors such as cytokines, oxidized LDL, immune complexes, TLR agonists, and angiotensin, all acting through receptor signaling to activate PKC, PIP3 kinase, Akt, Scr, and MAP kinases. This, in turn, activates NADPH oxidase, which produces O₂—. that is reduced to H₂O₂ by superoxide dismutase. H₂O₂ induces redox signaling through Akt, Src, p38 MAP Kinase, JNK/SAPK, ERK, and PKC. Redox sensitive transcription factors are activated, including NFκB, AP1, p53, Ets1, and HIF1α.

FIG. 9A through FIG. 9C depict the results of experiments demonstrating that HRGECs cultured with SLE and LN serum from patients with active disease induced increased neutrophil adherence over controls (FIG. 9A and FIG. 9B) and also increased neutrophil adherence over serum from the same SLE patients during inactive disease (FIG. 9C). n=4-5 each group, ** p<0.01.

FIG. 10A through FIG. 10C depict the results of experiments where HRGEC were treated with serum from patients with and without lupus, lupus nephritis, and hypertension. Washed cells were incubated in medium that was collected and placed in a transwell. Labeled neutrophil migration to the lower chamber was quantitated. Greater chemotaxis was seen with lupus than control serum (FIG. 10A). Lupus nephritis and non-nephritis hypertensive lupus patients serum induced greater chemotaxis (reported as cells×10⁴) (FIG. 10B). Serum from volunteers with hypertension, whether they had lupus or not, induced greater chemotaxis (FIG. 10C). * p<0.05; ** p<0.01; *** p<0.001.

FIG. 11 depicts the results of experiments illustrating eNOS dimer to monomer ratio in LN patients during inactive and active nephritis. Cell lysates from cells treated as in FIG. 9 were run on non-denaturing gel and stained for eNOS. N=5, *p>0.05.

FIG. 12 depicts the results of experiments demonstrating that NO production from LN-serum-treated HRGEC improved with sepiapterin more than VAS2870. HRGEC treated as in FIG. 9 were stained for DAF-FM (NO fluorescent probe) with urate to scavenge ONOO—. Sepiapterin added to culture increased NO production more than VAS2870 alone or in combination with sepiapterin.

FIG. 13 depicts the results of experiments illustrating eNOS ser1177 phosphorylation with control and inactive/active visit sera from SLE patients. Cell lysates from HUVEC treated as in FIG. 9 were run on a Western blot for peNOS-ser1177 and controlled eNOS stain intensity on the same blot after stripping. Subject numbers are included. C=Control, HTN=hypertension, Inactive/“−”=SLE visit from subject (ID shown) during remission, Active/“+”=SLE visit during flare. Stress=serum starved cells. Lower graph shows eNOS pSer1177 densities normalized to eNOS density.

FIG. 14 depicts the results of experiments illustrating HRGEC Sepiapterin Reductase (SPR) and Dihydrofolate Reductase (DHFR) expression with control and inactive/active visit sera from LN. Cell lysates from HRGEC treated as in FIG. 9 were divided in two and run on a Western blot for SPR (upper blot) and DHFR (middle blot) and controlled from β-actin (from SPR experiment shown in lower blot). Subject numbers are included. C=Control, HTN=hypertension, Inactive/“−”=SLE visit from subject (ID shown) during remission, Active/“+”=SLE visit during flare.

FIG. 15 depicts the results of experiments where 129 mice are aged to 10 weeks, and baseline urine is collected. Mice are injected with anti-GBM antibodies at 10 weeks, and nephritis is allowed to develop over two weeks with urine collections at 1 and 2 weeks. Dipstick urines are performed to confirm onset of nephritis. Mice are injected with enhanced GFP (eGFP) spleen cells (2×10⁶) and aged another 18 hours. Tissue and serum will be harvested.

FIG. 16 depicts the results of experiments where mice are aged until proteinuria above “trace” occurs in 1 mouse. Mice are divided into 2 groups: 1) 10 mg/day of sepiapterin and 2) vehicle. 24-hour urines is collected every 2 weeks for biomarker endpoints. When 75% of mice in any group develop proteinuria≥300 mg/dL, mice are injected with eGFP spleen cells and tissue is harvested 18 hours later.

FIG. 17 depicts the results of experiments where mice are aged until proteinuria≥300 mg/dL occurs in each mouse. Mice at the onset of proteinuria are treated with MMF and randomized into 2 groups: 1) 10 mg/kg/day of sepiapterin or 2) vehicle. 24-hour urine collections occur every 2 weeks for biomarker endpoints. When 75% of mice in any group have resolution of proteinuria, ≤30 mg/dL, mice are injected with eGFP spleen cells and tissue is harvested 18 hours later.

FIG. 18 depicts the results of experiments where mice are aged until proteinuria≥300 mg/dL occurs in each mouse. Mice at the onset of proteinuria are treated with standard-of-care MMF 100 mg/kg/day. At the time of resolution of proteinuria, MMF is withdrawn, and mice are sequentially randomized to receive either sepiapterin (10 mg/kg/day) or vehicle (n=15 per group). 24-hour urine collections occur at baseline and every 2 weeks after MMF withdrawal for biomarker endpoints. When 75% of mice in any group develop proteinuria≥300 mg/dL, mice are injected with eGFP spleen cells and tissue is harvested 18 hours later.

FIG. 19 depicts the results of experiments using HRGEC culture for RNA seq and protein phosphorylation. HRGEC are cultured with no serum, serum from healthy controls, or serum from LN patients during active flare and remission. Cells are washed and processed for RNA sequencing and protein phosphorylation assays.

FIG. 20 depicts the results of experiments using HRGEC culture for RNA seq and protein phosphorylation. HRGEC are cultured with serum from LN patients during active flare without added compounds or with added sepiapterin or VASS2970 alone or in combination. Cells are washed and processed for RNA sequencing and protein phosphorylation assays.

FIG. 21 depicts results of experiments illustrating the Ccange in systolic blood pressure from Day 7 to Day 14 following treatment with L-sep.

FIG. 22 depicts the results of experiments illustrating systolic blood pressure from Day 1 to Day 14.

FIG. 23 depicts the results of experiments demonstrating that Sepiapterin improves endothelial function in angiotensin II model of hypertension.

FIG. 24A through FIG. 24C depicts the results of experiments demonstrating that SLE serum promotes neutrophil chemotaxis, which is enhanced with hypertension (HTN), lupus nephritis (LN) and disease activity. (FIG. 24A) Neutrophil migration towards conditioned media from HRGECs treated with control (control, control HTN) or SLE with active disease (SLE, SLE HTN, SLE LN HTN) serum or (FIG. 24B) migration of neutrophils to conditioned media from HRGECs treated with serum from donor patient groups: control, control with HTN, SLE with active disease, SLE with HTN with active disease and SLE with LN and HTN with active disease. (FIG. 24C) Migration of neutrophils to conditioned media from HRGECs treated with serum from patients with SLE during paired inactive and active disease visits. Lines represent median values. Results are representative of three experiments, with each experiment using a different healthy neutrophil donor. Statistical analysis was by two-tailed unpaired t-test (FIG. 24A), one-way analysis of variance with Tukey's multiple comparisons (FIG. 24B) and two-tailed paired t-test (FIG. 24C). ***P<0.001, **p<0.01, *p<0.05.

FIG. 25A through FIG. 25C depicts the results of experiments demonstrating that cytokines secreted by SLE serum-exposed human renal glomerular endothelial cells (HRGECs) correlate with neutrophil chemotaxis. Cytokine array of conditioned medium from HRGECs treated with SLE serum collected during inactive and active disease (n=7 for paired inactive and active disease visits) revealed chemotactic factors (FIG. 25A) platelet-derived growth factor-BB (PDGF-BB), (FIG. 25B) interleukin (IL)-15 and (FIG. 25C) IL-8 that correlated with neutrophil migration to the same conditioned medium. Results are representative of two experiments. Statistical analysis was by Spearman's correlation. *P<0.05.

FIG. 26 depicts the results of experiments depicting a correlation matrix of factors in conditioned medium from SLE from serum-treated human renal glomerular endothelial cells (HRGECs). The correlation between levels of all cytokines from conditioned medium in the experiment from FIG. 25 was determined and displayed as a correlation matrix. Blue boxes reflect positive correlations, and the darkness of the box reflects the r value. Similarly, red indicates a negative correlation, and darkness of the box reflects the r value calculated by Spearman's correlation. GM-CSF, granulocyte macrophage colony stimulating factor

FIG. 27A through FIG. 27C depicts the results of experiments demonstrating that SLE serum promotes neutrophil adhesion, which is enhanced with disease activity. (FIG. 27A) Neutrophil adhesion to human renal glomerular endothelial cells (HRGECs) treated with control (control, control hypertension (HTN)) or SLE with active disease (SLE, SLE HTN, SLE lupus nephritis (LN) HTN) serum or (FIG. 27B) adhesion of neutrophils to HRGECs treated with serum from donor patient groups: control, control with HTN, SLE with active disease, SLE with HTN with active disease and SLE with LN and HTN with active disease. (FIG. 27C) Adhesion of neutrophils to HRGECs treated with serum from patients with SLE during paired inactive and active disease visits. Lines represent median values. Results are representative of three experiments, with each experiment using a different healthy neutrophil donor. Statistical analysis was by two-tailed unpaired t-test (FIG. 27A), one-way analysis of variance with Tukey's multiple comparisons (FIG. 27B) and two-tailed paired t-test (FIG. 27C). ***P<0.001, **p<0.01.

FIG. 28A through FIG. 28C depicts the results of experiments demonstrating that disease activity did not lead to a difference in (FIG. 28A) PDGF-BB, (FIG. 28B) IL-15, and (FIG. 28C) IL-8 levels with respect to disease activity.

FIG. 29A through FIG. 29C depicts the results of experiments demonstrating that HTN did not lead to a difference in (FIG. 29A) PDGF-BB, (FIG. 29B) IL-15, and (FIG. 29C) IL-8 levels with respect to disease activity.

FIG. 30A through FIG. 30C depicts the results of experiments demonstrating that renal involvement did not lead to a difference in (FIG. 30A) PDGF-BB, (FIG. 30B) IL-15, and (FIG. 30C) IL-8 levels with respect to disease activity.

FIG. 31 depicts the results of experiments, providing a table of the 25 most relevant pathways, sorted by p-value, related to IL-8, PDGFBB and IL-15 involved in neutrophil migration.

FIG. 32A through FIG. 32D depicts the results of experiments investigating neutrophil migration at differing doses of (FIG. 32A) Plaquenil, (FIG. 32B) Prednisone, (FIG. 32C) Lisinopril, and (FIG. 32D) CellCept.

FIG. 33A through FIG. 33D, depicts the results of experiments investigating cell adherence at differing doses of (FIG. 33A) Plaquenil, (FIG. 33B) Prednisone, (FIG. 33C) Lisinopril, and (FIG. 33D) CellCept.

FIG. 34A and FIG. 34B, depicts the results of experiments investigating neutrophil migration among those using (FIG. 34A) ACE inhibitors/angiotensin receptor blockers or (FIG. 34B) immunosuppressives.

FIG. 35A and FIG. 35B depicts the results of experiments investigating cell adherence among those using (FIG. 35A) ACE inhibitors/angiotensin receptor blockers or (FIG. 35B) immunosuppressives.

### DETAILED DESCRIPTION

The present invention relates to compositions comprising 2-amino-6-[-2-hydroxypropanoyl]-7,8-dihydro-1H-pteridin-4-one (L-sepiapterin or L-sep), also referred to as S-(−)-2-Amino-7,8-dihydro-6-(2-hydroxy-1-oxopropyl)-4(1H)-pteridinone, for modulating nitric oxide productions and their use for treating chronic inflammatory autoimmune diseases and disorders. The invention is based, in part, on the unexpected discovery that treatment of endothelial cells with L-sepiapterin preserved their capacity to produce NO in SLE conditions.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Generally, the nomenclature used herein and the laboratory procedures in cell culture, molecular genetics, organic chemistry, and nucleic acid chemistry and hybridization are those well-known and commonly employed in the art. Standard techniques or modifications thereof are used for chemical syntheses and chemical analyses.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

As used herein, a “peptidomimetic” is a compound containing non-peptidic structural elements that is capable of mimicking the biological action of a parent peptide. A peptidomimetic may or may not comprise peptide bonds.

As used herein, the term “inflammatory” means relating to inflammation. The term “inflammation” refers to the process by which vascular tissues responds to harmful stimuli, such as pathogens, damaged cells, or irritants. “Inflammation includes, but is not limited to secretion of and response to inflammatory factors, e.g., inflammatory cytokines.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to compositions and methods for treating chronic inflammatory autoimmune diseases and disorders.

In another aspect, the invention provides a method for treating or preventing a chronic inflammatory autoimmune disease or disorder or a chronic inflammatory autoimmune-related disease or disorder. In one embodiment, the method comprises administering a composition comprising a stimulator of nitric oxide (NO) production to a subject in need thereof.

In one embodiment, the stimulator of nitric oxide (NO) production is at least one of the group consisting of a chemical compound, a protein, a peptide, a peptidomemetic, an antibody, a ribozyme, a small molecule chemical compound, a nucleic acid, a vector, an antisense nucleic acid molecule.

Exemplary chronic inflammatory autoimmune diseases and disorders that can be treated using the compositions and methods of the invention include, but are not limited to, systemic lupus erythematosus (SLE), lupus nephritis, autoimmunity-related cardiovascular disease (CVD) and atherosclerosis, hypertension, systemic sclerosis, celiac disease, inflammatory bowel disease, type 1 diabetes, glomerulonephritis, Crohn's disease, psoriatic arthritis, or rheumatoid arthritis.

In one embodiment method further comprises administering a second therapeutic agent. In certain embodiments, the second therapeutic agent is an immunosuppressive agent.

In one embodiment, the subject is a mammal. In another embodiment, the mammal is a human.

### Stimulators of Nitric Oxide Production

In one embodiment, the present invention provides a composition comprising a stimulator of nitric oxide (NO) for treating or preventing a chronic inflammatory autoimmune disease or disorder.

In one embodiment, the composition of the invention comprises a nucleic acid, a peptide, a small molecule chemical compound, a peptidomimetic, or any combination thereof.

In various embodiments, the stimulator of NO production is a small molecule. Small molecule chemical compounds that can stimulate NO production include, but are not limited to, 2-amino-6-[-2-hydroxypropanoyl]-7,8-dihydro-1H-pteridin-4-one (L-Sepiapterin), tetrahydrobiopterin (BH4), nitroglycerin (GTN), isosorbide dinitrate (ISDN), isosorbide mononitrate (IS-5N), amyl nitrite, sodium nitroprusside (SNP), pentaerythrityltetranitrate (PETN), s-nitrosoglutathione, nicorandil and molsidomine, derivatives thereof, and salts thereof.

Where tautomeric forms may be present for any of the small molecules described herein, each and every tautomeric form is intended to be included in the present invention, even though only one or some of the tautomeric forms may be explicitly depicted.

The invention also includes any or all of the stereochemical forms, including any enantiomeric or diasteriomeric forms of the compounds described. The recitation of the structure or name herein is intended to embrace all possible stereoisomers of compounds depicted. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

In one embodiment, the small molecule compound of the invention comprises an analog or derivative of a compound described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule compounds described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule compounds described herein or can be based on a scaffold of a small molecule compound described herein, but differing from it in respect to certain components or structural makeup, which may have a similar or opposite action metabolically. An analog or derivative of any of a small molecule compound in accordance with the present invention can be used to treat a chronic inflammatory autoimmune disease or disorder.

In one embodiment, the small molecule compounds described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

In one embodiment, the stimulator of NO production is L-sepiapterin, or an analog, derivative or salt thereof. Exemplary analogs or derivatives of L-sepiapterin include, but are not limited to, formulations as described in U.S. Patent Publication No. US20200010469A1, U.S. Patent Publication No. US20190308975A1, PCT Patent Publication No. WO2019232120A1, PCT Patent Publication No. WO2019232120A1, and U.S. Patent Publication No. US20180078557A1, each of which is incorporated by reference herein in its entirety.

When the stimulator of NO is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule compound of the invention comprises an organic molecule, inorganic molecule, biomolecule, synthetic molecule, and the like.

### Combinations

In one embodiment, the composition of the present invention comprises a combination of a stimulator of NO production and a second therapeutic agent. Treatments for chronic inflammatory autoimmune diseases include immunosuppressant compounds. Additional immunosuppressive agents that can be administered in combination with a stimulator of NO production include, but are not limited to, corticosteroids (e.g., prednisone, budesonide, and prednisolone), oral disease modifying agents (e.g., sulfasalazine and hydroxychloroquine), tofacitinib, calcineurin inhibitors (e.g., tacrolimus and cyclosporine), antiproliferative agents (e.g., mycophenolate mofetil, mycophenolate sodium, leflunomide and azathioprine), mTOR inhibitors (e.g., sirolimus and everolimus), biologics (e.g., abatacept, adalimumab, anakinra, belimumab, certolizumab, etanercept, golimumab, infliximab, ixekizumab, natalizumab, rituximab, secukinumab, tocilizumab, ustekinumab, and vedolizumab) and other monoclonal antibodies (e.g., basiliximab, daclizumab, and muromonab)

In certain embodiments, a composition comprising a combination of a stimulator of NO and one or more additional therapeutic agent described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of compounds comprises individual compounds in any suitable ratio. For example, in one embodiment, the composition comprises a 1:1 ratio of two individual compounds. However, the combination is not limited to any particular ratio. Rather any ratio that is shown to be effective is encompassed.

### Therapeutic Methods

The present invention also provides methods of treating or preventing a chronic inflammatory autoimmune disease or disorder in a subject. In one embodiment, Chronic inflammatory autoimmune diseases and disorders that can be treated using the compositions and methods of the invention include, but are not limited to, SLE, lupus nephritis, autoimmunity-related CVD and atherosclerosis, hypertension, systemic sclerosis, celiac disease, inflammatory bowel disease, type 1 diabetes, glomerulonephritis, Crohn's disease, psoriatic arthritis and rheumatoid arthritis.

In one embodiment, the method comprises administering a composition of the invention to a subject who has a chronic inflammatory autoimmune disease or disorder. In one embodiment, the subject has a chronic inflammatory autoimmune disease or disorder selected from SLE, lupus nephritis, autoimmunity-related CVD and atherosclerosis, hypertension, systemic sclerosis, celiac disease, inflammatory bowel disease, type 1 diabetes, glomerulonephritis, Crohn's disease, psoriatic arthritis, and rheumatoid arthritis.

In one embodiment, the method comprises administering a composition of the invention to a subject who has an active chronic inflammatory autoimmune disease or disorder. In one embodiment, the method comprises administering a composition of the invention to a subject who is experiencing a flare of a chronic inflammatory autoimmune disease or disorder. In general, a flare is associated with a return of symptoms or an increase in severity of at least one symptom of the disease or disorder. However, some flares occur without symptoms. Common symptoms that indicate a flare include ongoing fever not due to an infection, painful or swollen joints, fatigue, rashes or sores or ulcers in the mouth or nose.

It will be appreciated by one of skill in the art, when armed with the present disclosure including the methods detailed herein, that the invention is not limited to treatment of a chronic inflammatory autoimmune disease or disorder that is already established. Particularly, the disease or disorder need not have manifested to the point of detriment to the subject; indeed, the disease or disorder need not be detected in a subject before treatment is administered. That is, significant signs or symptoms of a chronic inflammatory autoimmune disease or disorder do not have to occur before the present invention may provide benefit. Therefore, the present invention includes a method for preventing a chronic inflammatory autoimmune disease or disorder, in that a composition, as discussed elsewhere herein, can be administered to a subject prior to the onset of a chronic inflammatory autoimmune disease or disorder, thereby preventing the chronic inflammatory autoimmune disease or disorder. In one embodiment, the composition, as discussed elsewhere herein, can be administered to a subject having an inactive chronic inflammatory autoimmune disease or disorder to prevent disease activation, or a flare.

One of skill in the art, when armed with the disclosure herein, would appreciate that the prevention of a chronic inflammatory autoimmune disease or disorder, encompasses administering to a subject a composition as a preventative measure against the development of, or progression of, a chronic inflammatory autoimmune disease or disorder.

The invention encompasses administration of a stimulator of NO production for the treatment or prevention of a chronic inflammatory or autoimmune disease or disorder. In one embodiment, the stimulator of NO production is a small molecule chemical compound. In some embodiments, the small molecule chemical compound is 2-amino-6-[-2-hydroxypropanoyl]-7,8-dihydro-1H-pteridin-4-one (L-Sepiapterin), tetrahydrobiopterin (BH4), nitroglycerin (GTN), isosorbide dinitrate (ISDN), isosorbide mononitrate (IS-5N), amyl nitrite, sodium nitroprusside (SNP), pentaerythrityltetranitrate (PETN), s-nitrosoglutathione, nicorandil and molsidomine, derivatives thereof, and salts thereof. In one embodiment, the method comprises administering an effective amount of a composition comprising L-sepiapterin or an analog, derivative or salt thereof.

To practice the methods of the invention; the skilled artisan would understand, based on the disclosure provided herein, how to formulate and administer the appropriate composition to a subject. The present invention is not limited to any particular method of administration or treatment regimen.

One of ordinary skill in the art will appreciate, based on the disclosure provided herein, that the stimulator of NO production of the invention can be used to prevent or to a chronic inflammatory autoimmune disease or disorder, and that a stimulator of NO production composition can be used alone or in any combination with another immunosuppressive agent to effect a therapeutic result.

Additional immunosuppressive agents include, but are not limited to corticosteroids (e.g., prednisone, budesonide, and prednisolone), tofacitinib, calcineurin inhibitors (e.g., tacrolimus and cyclosporine), antiproliferative agents (e.g., mycophenolate mofetil, mycophenolate sodium, leflunomide and azathioprine), mTOR inhibitors (e.g., sirolimus and everolimus), biologics (e.g., abatacept, adalimumab, anakinra, certolizumab, etanercept, golimumab, infliximab, ixekizumab, natalizumab, secukinumab, tacilizumab, ustekinumab, and vedolizumab) and monoclonal antibodies (e.g., basiliximab, daclizumab, and muromonab).

One of skill in the art will appreciate that the compounds of the invention can be administered singly or in any combination. Further, compounds of the invention can be administered singly or in any combination in a temporal sense, in that a combination of a stimulator of NO production and a second therapeutic agent may be administered concurrently, or before, and/or after each other.

In one embodiment, the invention includes a method comprising administering a combination of compounds described herein. In certain embodiments, the method has an additive effect, wherein the overall effect of the administering a combination of a stimulator of NO production and a second therapeutic agent is approximately equal to the sum of the effects of administering each individual compound. In other embodiments, the method has a synergistic effect, wherein the overall effect of administering a combination of a stimulator of NO production and a second therapeutic agent is greater than the sum of the effects of administering each individual compound.

The method comprises administering a combination of a stimulator of NO production and a second therapeutic agent in any suitable ratio. For example, in one embodiment, the method comprises administering a stimulator of NO production and a second therapeutic agent at a 1:1 ratio. However, the method is not limited to any particular ratio. Rather any ratio that is shown to be effective is encompassed.

### Pharmaceutical Compositions and Formulations

The invention also encompasses the use of pharmaceutical compositions of the invention or salts thereof to practice the methods of the invention. Such a pharmaceutical composition may consist of at least one stimulator of NO production composition of the invention or a salt thereof in a form suitable for administration to a subject, or the pharmaceutical composition may comprise at least one modulator composition of the invention or a salt thereof, and one or more pharmaceutically acceptable carriers, one or more additional ingredients, or some combination of these. The compound or conjugate of the invention may be present in the pharmaceutical composition in the form of a physiologically acceptable salt, such as in combination with a physiologically acceptable cation or anion, as is well known in the art.

Exemplary stimulators of NO production that can be included in a pharmaceutical composition of the invention include, but are not limited to 2-amino-6-[-2-hydroxypropanoyl]-7,8-dihydro-1H-pteridin-4-one (L-Sepiapterin), tetrahydrobiopterin (BH4), nitroglycerin (GTN), isosorbide dinitrate (ISDN), isosorbide mononitrate (IS-5N), amyl nitrite, sodium nitroprusside (SNP), pentaerythrityltetranitrate (PETN), s-nitrosoglutathione, nicorandil and molsidomine, derivatives thereof, and salts thereof.

In an embodiment, the pharmaceutical compositions useful for practicing the methods of the invention may be administered to deliver a dose of between 1 ng/kg/day and 500 mg/kg/day. In another embodiment, the pharmaceutical compositions useful for practicing the invention may be administered to deliver a dose of between 100 ng/kg/day and 100 mg/kg/day. In another embodiment, the pharmaceutical compositions useful for practicing the invention may be administered to deliver a dose of between 500 ng/kg/day and 50 mg/kg/day. In another embodiment, the pharmaceutical compositions useful for practicing the invention may be administered to deliver a dose of between 5 mg/kg/day and 50 mg/kg/day. In another embodiment, the pharmaceutical compositions useful for practicing the invention may be administered to deliver a dose of between 10 mg/kg/day and 30 mg/kg/day. In another embodiment, the pharmaceutical compositions useful for practicing the invention may be administered to deliver a dose of between 15 mg/kg/day and 25 mg/kg/day. In another embodiment, the pharmaceutical compositions useful for practicing the invention may be administered to deliver a dose of about 20 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Accordingly, in one embodiment, the invention features a pharmaceutical composition including a stimulator of NO production, or a pharmaceutically acceptable salt, wherein the pharmaceutical composition is stable at room temperature (e.g., 25° C. or 25° C. and 60% relative humidity) for at least 6 months. For example, the level of the stimulator of NO production, or a pharmaceutically acceptable salt, in the composition decreases by less than 5% (e.g., less than 4%, less than 3%, less than 2%, less than 1%) when the composition is stored at room temperature for 6 months.

In some embodiments, the pharmaceutical composition is a solid composition (e.g., a powder, a capsule, or a tablet). In some embodiments, the pharmaceutical composition is formulated for use in a suspension. In some embodiments, the pharmaceutical composition further comprises a stabilizing agentin an amount sufficient to stabilize the stimulator of NO production, or a pharmaceutically acceptable salt thereof, in the composition for at least 6 months at room temperature (e.g., 25° C. or 25° C. and 60% relative humidity).

In one embodiment, the invention features a solid pharmaceutical composition of at least one stimulator of NO production, or a pharmaceutically acceptable salt thereof, and an antioxidant. In some embodiments, the antioxidant is present in an amount sufficient to stabilize the stimulator of NO production, or a pharmaceutically acceptable salt thereof, in the composition for at least 6 months at room temperature (e.g., 25° C. or 25° C. and 60% relative humidity).

In an aspect, the invention features a pharmaceutical composition including at least one stimulator of NO production, or a pharmaceutically acceptable salt thereof, and a stabilizing agent, wherein the ratio of the at least one stimulator of NO production, or a pharmaceutically acceptable salt thereof, to stabilizing agent is greater than 4:1 (e.g., greater than 5:1, greater than 6:1, greater than 7:1, greater than 8:1, greater than 9:1, greater than 10:1, greater than 15:1, greater than 20:1) wt/wt.

In an aspect, the invention features a pharmaceutical composition including at least one stimulator of NO production, or a pharmaceutically acceptable salt thereof, and an antioxidant (e.g., ascorbic acid), wherein the pharmaceutical composition includes more of the at least one stimulator of NO production, or pharmaceutically acceptable salt thereof, than antioxidant by weight. For example, in some embodiments, the at least one stimulator of NO production, or pharmaceutically acceptable salt thereof, and antioxidant (e.g., ascorbic acid) are present in a ratio of at least 1:1 (e.g., 2:1, 3:1, 4:1, 5:1, 6:1, 7:1, 8:1, 9:1, or 10:1) wt/wt.

Exemplary antioxidants that can be included in the compositions of the invention include, but are not limited to, 4-chloro-2,6-di-tert-butylphenol, tocopherol, alpha-tocopherol, alkylated diphenylamines, ascorbic acid, ascorbyl myristate, ascorbyl palmitate, ascorbyl stearate, beta-carotene, butylated hydroxyanisole, butylated hydroxytoluene, citric acid, cysteine, D-alpha-tocopheryl polyethylene glycol 1000 succinate, deferoxamine methanesulfonate, dodecyl gallate, ethylparaben, folic acid, fumaric acid, gallic acid, glutathione, lecithin, malic acid, methylparaben, monothioglycerol, N-acetyl cysteine, nordihydroguaiaretic acid, octyl gallate, p-phenylenediamine, potassium ascorbate, potassium metabisulfite, potassium sorbate, propionic acid, propyl gallate, retinol, sorbic acid, sodium ascorbate, sodium bisulfite, sodium hydrosulfite, sodium isoascorbate, sodium metabisulfite, sodium sulfite, sodium thiosulfate, tartaric acid, tert-butylhydroquinone, tocopheryl acetate, vitamin A, vitamin B6, vitamin B12, or vitamin E, or a combination thereof. In some embodiments of any of the foregoing pharmaceutical compositions, the antioxidant is ascorbic acid, tocopherol, retinol, ascorbyl palmitate, N-acetyl cysteine, glutathione, butylatedhydroxytoluene, and/or butylatedhydroxyanisole.

In some embodiments, the invention features a particulate pharmaceutical composition including at least one stimulator of NO production, or a pharmaceutically acceptable salt thereof, for use in a suspension.

In some embodiments, the invention features a pharmaceutical composition formulated as a suspension in a dosing vehicle including a bulking agent or anti-caking agent, wherein at least 50% (e.g., at least 60%, at least 70%, at least 80%, at least 90%, at least 95%, or at least 99%) of the at least one stimulator of NO production, or pharmaceutically acceptable salt thereof, is dissolved in the dosing vehicle.

In some embodiments, the concentration of at least one stimulator of NO production, or a pharmaceutically acceptable salt thereof, in the dosing vehicle is 1 mg/mL to 5 mg/mL (e.g., at least 1 mg/mL, at least 1.1 mg/mL, at least 1.2 mg/mL, at least 1.3 mg/mL, at least 1.4 mg/mL, at least 1.5 mg/ml, at least 1.6 mg/mL, at least 1.7 mg/mL, at least 1.8 mg/mL, at least 1.9 mg/mL, at least 2.0 mg/mL, or at least 2.1 mg/ml).

In some embodiments, the invention features a pharmaceutical composition formulated as a suspension in a dosing vehicle including a bulking agent or anti-caking agent, wherein at least 50% (e.g., at least 60%, at least 70%, at least 80%, at least 90%, at least 95%, or at least 99%) of the at least one stimulator of NO production, or a pharmaceutically acceptable salt thereof, is adsorbed on the bulking or anti-caking agent.

In some embodiments, the pharmaceutical composition includes about 20-95% (e.g., about 20%, about 21%, about 22%, about 23%, about 24%, about 25%, about 26%, about 27%, about 28%, about 29%, about 30%, about 40%, about 50%, about 60%, about 70%, about 80%, about 90%, about 95% or about 20-30%, about 25-45%, about 40-60%, about 50-75%, about 65-75%, about 70-90%, about 85-95%) of the stimulator of NO production, or a pharmaceutically acceptable salt thereof, by total weight. In some embodiments of any of the foregoing compositions, the pharmaceutical composition further includes a dispersant (e.g., a carboxymethylcellulose or a pharmaceutically acceptable salt thereof). In some embodiments of any of the foregoing compositions, the pharmaceutical composition includes 0.1-1.5% (e.g., 0.1-0.3%, 0.2-0.4%, 0.3-0.5%, 0.4-0.6%, 0.5-0.7%, 0.6-0.8%, 0.7-0.9%, 0.8-1%, 0.9-1.1%, 1-1.2%, 1.1-1.3%, 1.2-1.4%, or 1.3-1.5%) dispersant by total weight.

In some embodiments, the pharmaceutical composition includes at least one anti-caking agent or bulking agent (e.g., a bulking agent and an anti-caking agent). In some embodiments, the at least one anti-caking agent or bulking agent is colloidal silicon dioxide or microcrystalline cellulose. In some embodiments, the pharmaceutical composition includes 60-80%, e.g., 65-75%, (e.g., about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%) anti-caking agent and/or bulking agent by total weight, in some embodiments of any of the foregoing compositions, the pharmaceutical composition includes both colloidal silicon dioxide and microcrystalline cellulose, in some embodiments of any of the foregoing compositions, the pharmaceutical composition includes 60-65% (about 60%, about 61%, about 62%, about 63%, about 64%, or about 65%) microcrystalline cellulose by total weight and 2-15% (e.g., about 5-7%, about 2%, about 3%, about 4%, about 5%, about 6%, about 7%, about 8%, about 9%, about 10%, about 11%, about 12%, about 13%, about 14%, or about 15%) colloidal silicon dioxide by total weight.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples therefore, are not to be construed as limiting in any way the remainder of the disclosure.

### Example 1: L-Sepiapterin Restores SLE Serum-Induced Markers of Endothelial Function in Endothelial Cells

Multiple studies have shown that conventional cardiovascular disease risk factors cause an increase rather than decrease in eNOS expression (Guzik, T. J. et al., 2002, Circulation, 105:1656-62; Hink, U. et al., 2001, Circ. Res., 88:E14-22). Moreover, while mechanisms involved in SLE serum induced changes in eNOS gene expression are unknown, SLE serum contains large quantities of Crib receptor ligands, including anti-endothelial cell and double-stranded DNA autoantibodies (van der Zee, J. M. et al., 1991, Clin. Exp. Immunol., 84:238-44). Previous studies demonstrated that FcγRIIB receptor engagement yields disruption in eNOS enzymatic activity and NO production (Mineo, C. et al., 2005, Circ, Res., 97:1124-31; Venugopal, S. K. et al., 2002, Circulation, 106:1439-41). Whether these factors trigger activation of pathways that lead to compensatory increases eNOS expression is unknown.

Interestingly, SLE patient age negatively correlated with eNOS mRNA expression. Chronological age is an important non-modifiable cardiovascular risk factor in SLE and non-SLE populations. The relationship between SLE patient age and in vitro endothelial cell eNOS mRNA expression is paradoxical due to conflicting results showing an overall increase in eNOS mRNA expression in SLE. Age is associated with rises in oxidative stress which contributes to progressive declines in endothelium-dependent vasodilation associated with diminished NO bioavailability. Moreover, with age, traditional risk factors including hypertension and dyslipidaemia increase in prevalence (Erdozain, J. G. et al., 2017, Lupus Sci. Med., 4:e000190). Without being bound by theory, it was postulated that inflammatory cytokines such as IFN-α, the presence of oxidative stress and the prevalence of conventional cardiovascular disease risk factors could work synergistically to suppress eNOS mRNA expression with age in patients with SLE.

The role of NO in lupus is multifaceted and oppositional in specific clinical manifestations of the disease. Recent work has suggested that the imbalance between [NO]/[ONOO⁻] significantly contributes to the development of endothelial dysfunction (Huang, J. et al., 2008, J. Vasc. Surg., 47:599-607). In SLE, it is postulated that this imbalance stems from a non-resolving inflammatory response (van der Zee, J. M. et al., 1991, Clin. Exp. Immunol., 84:238-44; Mineo, C. et al., 2005, Circ, Res., 97:1124-31; Venugopal, S. K. et al., 2002, Circulation, 106:1439-41; Denny, M. F. et al., 2007, Blood, 110:2907-15) that leads to impaired eNOS protein expression and activation, enhanced ROS generation, conversion of BH₄ to BH₂ and subsequent perpetuating of eNOS uncoupling (Kuzkaya, N. et al., 2003, J. Biol. Chem., 278:22546-54). In the current study, L-sep preserved NO production in response to SLE serum. The preservation of NO production after L-sep treatment may have resulted from increased de novo BH₄ synthesis; still, future studies are needed to assess L-sep-mediated preservation of NO. However, a rise in endogenous NO production failed to prevent neutrophil adhesion. These findings contradict the established concept that NO can reduce adhesion molecule expression (Kubes, P. et al., 1991, Proceedings of the National Academy of Sciences, 88:4651-5). The reason for this discrepancy is likely multifactorial including the fact that mechanisms mediated by NO are concentration dependent and NO production quantitatively was assessed but rather qualitatively. Moreover, this studies relied on the use of endogenous rather than exogenous NO previously shown to prevent oxidation of LDL (Maggi, M. F. et al., 1995, C. R. Seances Soc. Biol. Fil., 189:375-87), smooth muscle cell migration (Gorog, P. et al., 1998, J. Vasc. Res., 35:165-9) and adhesion molecule expression (Spiecker, M. et al., 1997, J. Biol. Chem., 272:30969-74). Still, these studies did not assess the effectiveness of exogenous NO under opposing inflammatory conditions. These findings are more consistent with in vitro data showing increases in eNOS gene expression coupled with losses in NO production are indicative of dysfunctional eNOS which produces higher levels of superoxide (Kawashima, S. et al., 2004, Endothelium, 11:99-107). Peroxynitrite, a molecule generated through the interaction of NO and superoxide, oxidizes BH₄ to BH₂ which is not adequate for eNOS enzymatic activity or production of biologically effective concentrations of NO. Thus, oxidation of de novo BH₄ could also serve as a potential mechanism of impaired NO bioavailability. Alternatively, mitochondrial dysfunction may contribute to losses in viable BH₄.

Endothelial cells have a very low, mitochondrial content and predominately rely on glycolysis for glucose oxidation and fatty acid oxidation flux (Groschner, L. N. et al., 2012, Pflügers Archiv.—European Journal of Physiology, 464: 63-76). As a result, endothelial cells produce lower levels of oxphos-generated ROS and adjust more readily to hypoxic environments (Ghesquiere, B. et al., 2014, Nature, 511:167-76). Under certain conditions, however, mitochondria become dysfunctional and produce higher levels of ROS. In these studies, the basal OCR increased in response to human serum (both control and SLE). It is postulated that this is expected given the increase in fatty acids and glucose introduced into the cell culture environment. Moreover, no differences were observed in bioenergetic health indices between human serum groups, which suggests that SLE serum-specific contents do not induce mitochondrial dysfunction and that mitochondrial oxidative stress was not likely the cause of reductions in bioavailable NO in the SLE group.

This study demonstrated that SLE serum cultured endothelial cells have an increased capacity to induce migration of neutrophils to the endothelial cell surface and bind neutrophils compared with control serum or buffer cultured cells. These findings suggest that factors present in SLE serum may induce adhesion molecule expression, possibly through diminished NO. Previous studies suggest that diminished NO bioavailability further promotes leucocyte adhesion to the endothelial cell surface as NO is known to be a modulator of adhesion and emigration (Kubes, P. et al., 1991, Proceedings of the National Academy of Sciences, 88:4651-5; Tiefenbacher, C. P. et al., 2003, Pflugers Arch, 447:1-7). Surprisingly, supplementation of cultures with L-sep did not reverse neutrophil adhesion to the endothelial cell surface induced by SLE serum. This suggests that other factors present in SLE serum, including cytokines and chemokines, may promote adhesion molecule expression that cannot be overcome by endogenous NO production in an in vitro cell culture model.

The materials and methods employed in these experiments are now described.

### Patient Population

Specimens for this study were stored and collected from study visits that were part of a longitudinal observational cohort study known as the SLE Gullah Health or SLEIGH (Kamen, D. L., et al., 2008, Arthritis, Rheum., 58:1237-47). All patients classified as having SLE met 4 of the 11 classification criteria as specified by the 1997 American College of Rheumatology criteria (Hochberg, M. C., 1997, Arthritis. Rheum., 40:1725).

Clinical Characteristics of the Study Population

Patients with SLE were evaluated during regular clinical visits by clinicians trained in SLE disease activity measures. The clinical and laboratory elements of the SLE Disease Activity Index (SLEDAI) were recorded if they were attributed to SLE disease activity. The scores were recorded for each blood collection visit. Control volunteers were evaluated for autoimmune disease and the presence of cardiovascular disease or risk factors (hypertension, smoking, hypercholesterolemia or previous myocardial infarction, cardiac or brain revascularization, or stroke).

Blood Collection

Blood from healthy and participants with SLE were collected in a sterile vacutainer blood collection tube and whole blood was allowed to clot at 25° C. for 10 min. Samples were centrifuged to remove the clot and remaining serum was stored in aliquots at −80° C. for future use.

Neutrophil Isolation

Neutrophils were isolated as previously described (Oh, H. et al., 2008, Journal of Visualized Experiments). Briefly, 20 mL of human blood was acquired from healthy volunteers and cells were isolated using Lymphocyte Separation Medium (Cellgro, Manassas, Va., USA). The assay was validated based on forward scatter and side scatter using flow cytometry.

Endothelial Cell Culture and Serum Culturing

Primary human umbilical vein endothelial cells from pooled donors were purchased from Lonza (Walkersville, Md., USA) and cultured according to the manufacturer's instructions in 5% CO₂ at 37° C. in humidified air. Cells were cultured in endothelial cell basal medium-2 (EBM-2) supplemented with EBM-2 SingleQuot (Lonza), pH 7.6-8.0. Media was changed every other day until cells were 70%-80% confluent. HUVECs were sub-cultured using TryPLE Express, pH 8.0 (ThermoFisher Scientific, Waltham, Mass., USA). Cell growth was limited to 12 population doublings, and all experiments were carried out using cells between passages 3-5. For serum experiments, cells were cultured with 20%-50% serum for 6 or 24 hours prior to further analysis.

Real-Time Reverse Transcription-PCR (RT²PCR)

To detect changes in mRNA levels, HUVECs were treated with 20% SLE or healthy sera for 6 or 24 hours as specified. Cells were harvested following treatment and total RNA was extracted using a Trizol (ThermoFisher Scientific)-RNeasy kit (Qiagen, Frederick, Md., USA) hybrid protocol as previously described (Haimov-Kochman, R. et al., 2006, Clin. Chemi., 52:159-60). RNA integrity was assessed using a NanoDrop 2000c UV-Vis spectrophotometer (ThermoScientific, Wilmington, Del., USA) and samples with A_{260/280} ratios of 1.8-2.1 were used. Single strand cDNA was synthesised from 1 μg of RNA using an iScript cDNA synthesis kit (Bio-Rad, Hercules, Calif., USA). For each reaction, 1 μL of cDNA product was used for signal amplification with SsoAdvanced universal SYBR (BioRad). A CFX96 Real Time PCR Detection System (Bio-Rad) was used to assess changes in NOS3 and GAPDH, using commercially available primers (Qiagen). The relative expression was calculated using the equation 2^{-ΔΔCt} (Δ; experimental gene cycle threshold (Cₜ)-housekeeping gene (Cₜ)). The fold change gene expression of interest was calculated based on normalization to GAPDH. PCR was performed≥3 independent experiments with at least three replicates.

Measurement of Nitric Oxide Production

For real-time detection of NO production in HUVECs, 1.2×10⁵ cells were seeded in a 12-well tissue culture plate. Following adherence, cells were serum starved for 6 hours in endothelial basal media (EBM) containing 0.2% fetal bovine serum (FBS). Cells were stimulated with either 50% healthy or SLE sera±L-sep (5 μM; 6 hours), the eNOS-specific inhibitor, Nω-Nitro-L-arginine (L-NNA, 10 μM; 30 min pre-incubation (Tocris; Bristol, UK)) or the NO donor 3,3′-diamino-4′-methoxyflavone (DD1, 10 μM, Tocris). Following stimulation, cells were washed twice with phosphate buffered saline (PBS) and loaded with 1 μM DAF-FM diacetate (4-amino-5-methylamino-2′,7′-difluorofluorescein diacetate, 1 μM) (ThermoFisher Scientific) in phenol red-free EBM for 30-45 min. Cells were washed twice with PBS and dissociated from plates using phenol-red free TryPLE Express (ThermoFisher Scientific) and fixed with 2% paraformaldehyde for 3 min. A population of 2000-10 000 cells were gated to remove doublets and controls and analyzed based on their fluorescence intensities using a FACS Calibur flow cytometer (Becton Dickenson, San Diego, USA). The mean fluorescence intensity (MFI) was normalized to respective populations in unstimulated cells. In order to discriminate between NO and other gaseous molecules previously shown to augment DAF-FM fluorescence, a urate assay was performed to optimize the assay (data not shown).

Oxygen Consumption

Endothelial cells were seeded at 20,000 cells/well on a Seahorse 96-well XF Cell Culture Microplate as detailed by the manufacturer (Seashore Bioscience/Agilent Technologies, Santa Clara, Calif., USA) and allowed to adhere overnight in complete EBM-2 (EBM-2 basal media plus EBM-2 SingleQuots, Lonza, Basel, Switzerland). The following day cells were rinsed with 1×PBS and 50% control or SLE patient serum was added to wells and allowed to incubate for 24 hours (six samples per group with five replicates per patient sample). The Seahorse XF Analyzer (Seashore Bioscience/Agilent Technologies) was used to determine basal oxygen consumption rate (OCR). Four basal rate measurements were followed by four measurement cycles following each injection (1 μM oligomycin, 1 μM carbonyl cyanide 4-(trifluoromethoxy) phenylhydrazone and 2 μM AA rotenone). Consumption rates were calculated as previously described (Beeson, C. C. et al., 2010, Anal Biochem., 404:75-81). The bioenergetic health index (BHI) was calculated using the following formula: BHI=(ATP-linked×Reserve Capcity)/(Proton Leak×Non-mitochondrial OCR) (National Center for Health Statistics (US), 2016, Health, United States, 2015: with special feature on racial and ethnic health disparities. Hyattsville (Md.): National Center for Health Statistics (US)).

Neutrophil Adhesion Assay

HUVECs were plated at 5.0×10⁴ cells/mL in a 24-well plate (Costar) and allowed to adhere overnight. HUVECs were serum starved for 3 hours in phenol-red free 0.2% FBS EBM media (Lonza) prior to activation with 10% sera for 4 hours. Tumor necrosis factor-α (100 ng/mL) was used as the positive control. Neutrophils isolated from healthy human blood as outlined previously were labelled with Calcein AM (Life Technologies) at 5×10⁵ cells/mL. Neutrophils were washed gently four times in warm serum-free EBM culture media prior to co-culturing with HUVECs for 60 min after which non-adherent cells were removed by repeated gentle washing (four times) with EBM culture media. Fluorescence intensity was measured at 520 nM with a FLUOStar Omega microplate reader (Cary, N.C., USA), and images were captured using confocal microscopy. Data are reported as ratios of the number of neutrophils to the number of endothelial cells as averages from three different visual fields.

Neutrophil Migration Assay

Transwell migration assays were performed as described elsewhere (Jestus, C. R. et al., 2014, J. Vis. Exp.). Briefly, transwell inserts (3 μm pore) were pre-coated with fibrinogen and allowed to incubate overnight for 24 hours. HUVECs were seeded in 24-well plates at 1.0×10⁴ cells per well and allowed to adhere overnight. Cells were activated with 50% sera from healthy and SLE controls for 6 hours and washed once in PBS. Neutrophils were added to chambers and inserted into media±IL-8 (1.25 nM, Cell Signaling). After 60 min, the number of neutrophils in the lower chambers was visualized using 4× magnification and quantified using confocal microscopy. All values were normalized to untreated controls.

Statistical Analysis and Data Handling

Descriptive statistics are reported as mean±SD or IQR for continuous variables. Gaussian distribution was determined using the D'Agostino-Pearson omnibus normality test and the Shapiro-Wilk normality test. Paired and unpaired (where appropriate) two-tailed Student's t-test and non-parametric Mann-Whitney test were used on non-parametric data analysis on lupus and controls. Correlations were determined using Pearson's or Spearman's correlation analysis and are reported accordingly. Standardized univariate regression analysis was performed to adjust for lupus-associated indicators of disease activity and β-coefficients and p values were reported. Analysis of variance test with Tukey' Fisher's probable least significance post-test was used to analyses NO and neutrophil adhesion data. No mathematical correction was made for multiple comparisons. Data are presented as mean±SEM. Differences were considered significant if the p value was <0.05. Statistical analysis was performed using IBM SPSS Software V.25 or GraphPad Prism V.6.0f (San Diego, Calif., USA).

The results of the experiments are now described.

Demographic and Clinical Characteristics of Study Participants

SLE (n=25) and healthy controls (n=14) did not differ in sex, race or age. Mean±SD disease duration for patients with SLE was 9.4±6.4 and the age at diagnosis was 22.6±9.1. SLEDAI (Tial et al., 2016, J Am Acad Dermatol 2016; 74:862-9; Romero-Diaz et al., 2011, Arthritis Care Res 2011; 63(Suppl 11):537-46) was 4.6±3.5, reflecting overall mild lupus disease activity. Complement C3 levels were 104.7±37.4 while C4 levels were 24.3±13.8 further supporting the notion that patients with lupus in this study had mild disease activity. Patients with SLE had normal blood pressures and an overall body mass index of 26±5, indicating a group of participants that were slightly overweight. Most patients with SLE were taking prednisone and/or antimalarial medications (76%, 68%, respectively) at the time of the study visit (Table 1 and Table 2).

TABLE 1
Demographics

| | | |
|---|---|---|
| Healthy | SLE | P value |

Demographics

| | | | |
|---|---|---|---|
| All, n | 14 | 25 | |
| Age (years ± SD) | 33 ± 13 | 31 ± 6 | 0.5142 |

| | | | | | |
|---|---|---|---|---|---|
| Female sex, n (%) | 13 | (92%) | 23 | (92%) | 0.99 |

Race

| | | | | | |
|---|---|---|---|---|---|
| African-American | 12 | (86%) | 19 | (76%) | 0.6857 |
| Other | 2 | (14%) | 6 | (24%) | |

Values represent mean ± SD. A student's t-test was used for parametric data and a X² test was performed for categorical variables.

TABLE 2
Demographics and clinical characteristics
Lupus clinical characteristics

| Disease duration | 9.4 ± 6.4 | |
|---|---|---|
| Age at diagnosis | 22.6 ± 9.1 | |
| SLE Disease Activity Index | 4.6 ± 3.5 | |
| SLICC | 0.7 | (0.2-1.2) |
| Anti-dsDNA antibodies (mg/dL) | 123.7 ± 106.3 | |
| C3 complement (mg/dL) | 104.7 ± 37.4 | |
| C4 complement (mg/dL) | 24.3 ± 13.8 | |
| Urine creatinine (mg/dL) | 184 | (132-236) |
| Urine protein (mg/dL) | 123 | (85-161) |
| Urine protein/creatinine ratio | 0.5 | (0.2-0.7) |
| White blood cell count | 4.9 | (3.9-5.9) |

Medications

| | | |
|---|---|---|
| Hydroxychloroquine | 17 | (68%) |
| Azathioprine | 6 | (24%) |
| Methotrexate | 1 | (4%) |
| Humira | 1 | (4%) |
| Benlysta | 1 | (4%) |
| Mycophenalate mofetil | 10 | (40%) |
| Prednisone | 19 | (76%) |

General history

| | |
|---|---|
| Body mass index | 26 ± 5 |

Blood pressure

| | | |
|---|---|---|
| Systolic (mmHg) | 121 | (±18) |
| Diastolic (mmHg) | 73 | (±14) |

Serum from Patients with SLE Alters eNOS mRNA Expression

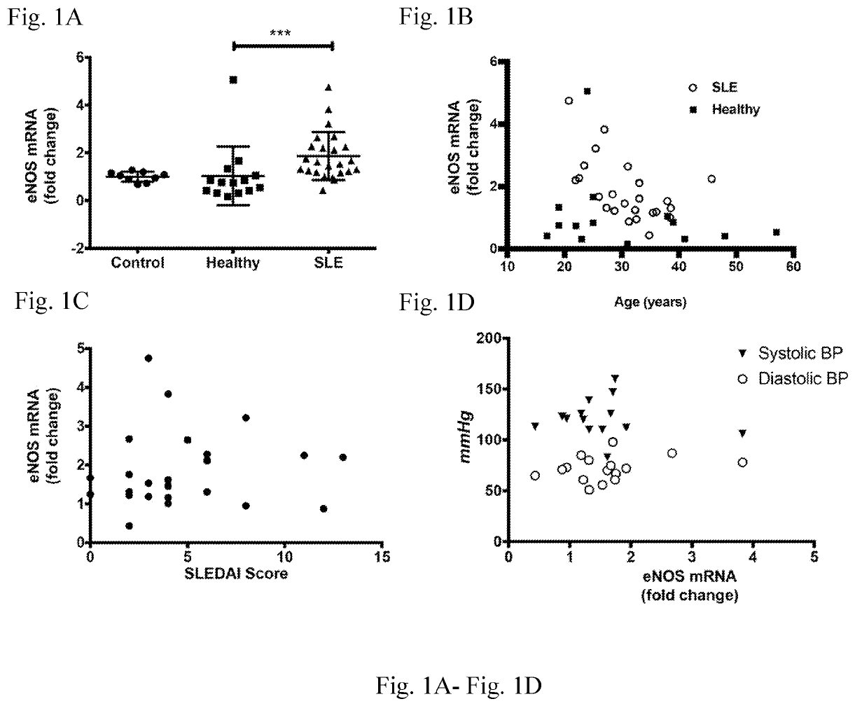

It was determined whether eNOS mRNA expression changed in primary HUVECs in response to 50% (v/v) serum of EBM-2, control patients or patients with SLE. SLE serum increased eNOS mRNA expression compared with healthy patient serum (1.88±0.22-fold, p<0.05; FIG. 1A). SLE patient age but not control patient age negatively correlated with eNOS mRNA expression in SLE serum cultured HUVECs (r=−0.59, p=−0.003; FIG. 1B). The correlation persisted in multivariate regression analysis adjusting for lupus disease activity as measured by SLEDAI (FIG. 1C), disease duration, dsDNA, and C3 and C4 levels (data not shown). None of these disease activity covariables themselves independently associated with eNOS mRNA expression. No significant correlation was found between eNOS mRNA and SLE patient blood pressures (FIG. 1D). No other correlations with eNOS mRNA expression were observed.

SLE Serum Reduced NO in Endothelial Cells

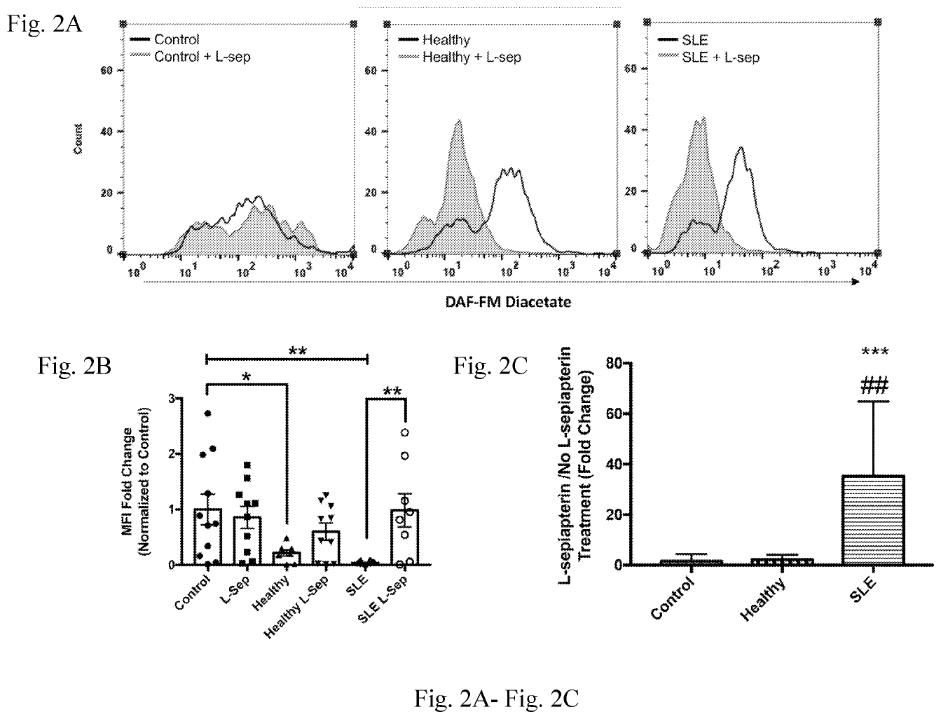

As shown in FIG. 2A and FIG. 2B, exposure to SLE serum resulted in reduced synthesis of intracellular basal NO as evidenced by reduced DAF-FM diacetate MFI (p<0.01; 23.6-fold) compared with EBM-2 cultured endothelial cells. Moreover, HUVEC exposure to healthy serum caused a reductions in NO compared with EBM-2 but these differences were not as pronounced as changes observed with SLE serum (p<0.05; 4.56-fold).

L-Sep Restores NO Production in SLE Serum Cultured Endothelial Cells

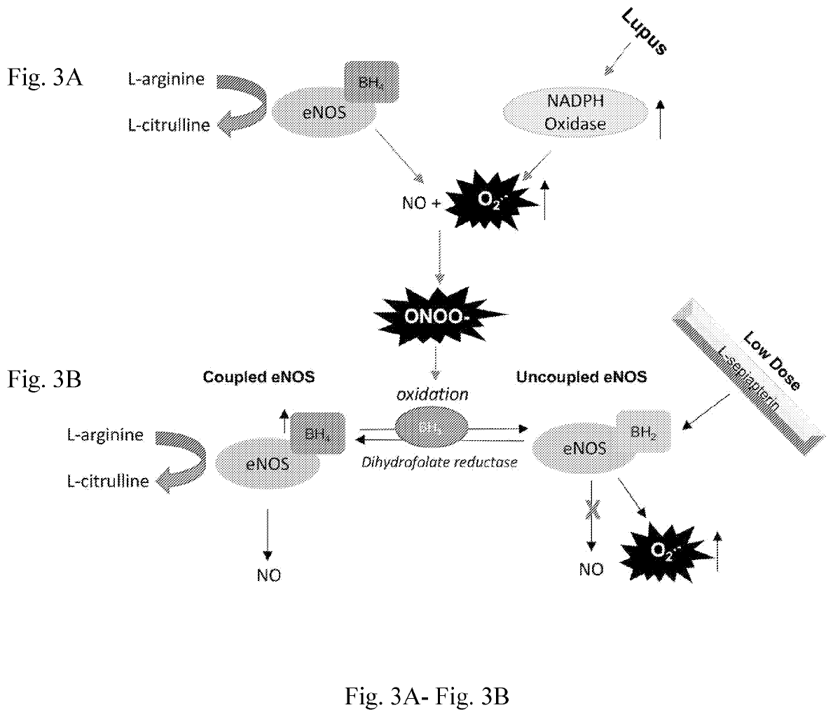

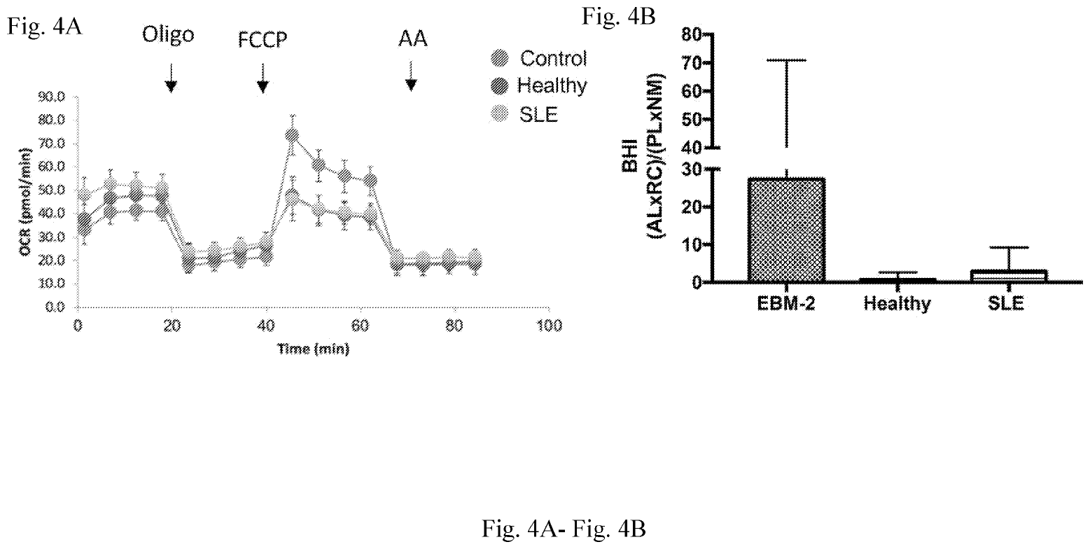

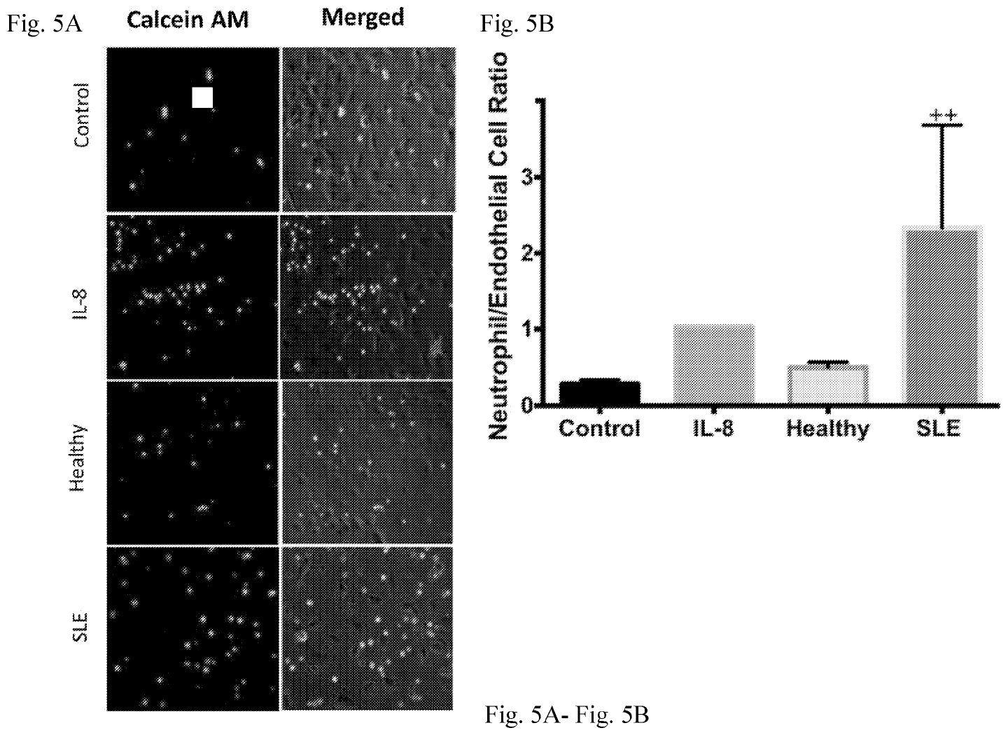

Treatment with L-sep, important for tetrahydrobiopterin synthesis (see FIG. 3), significantly increased DAF-FM MFI, a marker of NO production, in SLE serum cultured HUVECs compared with SLE cultured cells without L-sepiapterin (p<0.01; FIG. 2B). These changes were not observed in control serum cultured HUVECs. Moreover, it was observed that the ratio in MFI-fold change between SLE serum+L-sepiapterin and SLE serum was significantly different from ratios in control and healthy serum culture conditions (p<0.001 and <0.01, respectively; FIG. 2C).

SLE Serum does not Alter Cellular Bioenergetics in Endothelial Cells Compared with Healthy Serum

Mitochondrial dysfunction is reflective of changes in mitochondrial membrane potential, a reduction in the ATP level and the inhibition of mitochondrial OCR (Ren, J. et al., 2010, J. Mol. Med., 88:993-1001). In addition, excessive ROS contributes to mitochondrial dysfunction as assessed by increased maximal respiration (Zorov, D. B. et al., 2006, Biochimica et Biophysica Acta (BBA)-Bioenergetics, 1757:509-17). Thus, it was sought to determine whether cellular bioenergetics were impacted in response to SLE serum in order to determine the role of mitochondrial oxidative stress in diminished NO production. FIG. 4A illustrates the profile of endothelial cells from the three cell culture conditions (EBM-2, healthy serum, SLE serum) after 24 hours. The BHI has previously been shown to be a sensitive measure of oxidative stress and inflammation in various cell types (citation). On calculating the BHI (FIG. 4B), no statistically significant differences in BHI was found, indicating that changes in mitochondrial metabolism do not explain differences in NO production between treatment groups.

SLE Serum Induced Neutrophil Migration

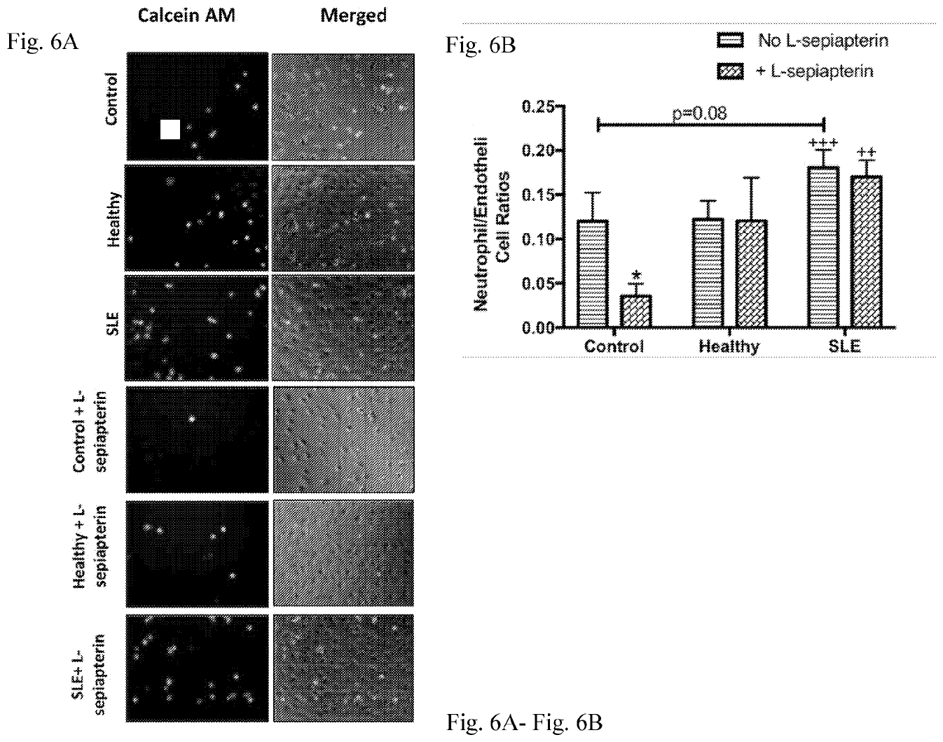

SLE serum (50% v/v) cultured HUVECs displayed an increased capacity for induction of neutrophil migration as assessed using flow cytometry and confocal microscopy compared with healthy control serum cultured cells (0.50±0.15 vs 1.00±0.54, p<0.01; FIG. 5A and FIG. 5B). SLE cultured cells also enhanced neutrophil adhesion to the endothelial cell surface compared with cells cultured in EBM-2 media but not healthy control serum. However, these differences were not statistically significant (p=0.07; FIG. 6A and FIG. 6B). It should be noted that 83% of the SLE patient serum samples used for these experiments were derived from patients who were taking stable doses of prednisone and at least one additional immunosuppressive therapy medication which may have impacted the robustness of neutrophil adherence to the endothelial cell surface under SLE culture conditions.

To determine whether the observed changes in neutrophil adhesion were due to eNOS uncoupling and subsequent declines in NO production, cell cultures were supplemented with L-sep to further understand the role of NO in the observed paradigm. L-sep reversed neutrophil adhesion in cell cultured in EBM-2 media (p<0.05); however, L-sep did not reverse neutrophil adhesion induced by SLE serum (FIG. 6A and FIG. 6B). These results suggest that other factors beyond NO might be responsible for changes in neutrophil adhesion to the endothelial cell surface.

In this study, it was observed that SLE serum greatly enhanced eNOS mRNA expression, a phenomenon negatively associated with age. In addition, it was observed that SLE serum diminished basal NO in endothelial cells and that L-sep restored levels to normal. While cellular bioenergetics contribute to changes in cell metabolism and signaling pathways, no difference was observed between the impact of SLE and healthy serum on cellular bioenergetics. Increased levels of neutrophil migration were observed under SLE serum versus healthy serum culture conditions. Moreover, neutrophil adhesion also increased; however, L-sep did not reverse the impacts of SLE on neutrophil adhesion to the endothelial cell surface. Together, these data provide the rationale for the hypothesis that preservation of eNOS function and subsequent NO production may play an important role in protecting against SLE-mediated endothelial dysfunction.

In summary, endothelial dysfunction is due, in part, to dysfunctional eNOS and diminished NO bioavailability. Lupus serves as an independent risk factor for endothelial dysfunction and contributes to accelerated atherosclerosis (El-Magadmi, M. et al., 2004, Circulation, 110:399-404). This data provide evidence for dysfunctional eNOS in vitro in the presence of SLE serum.

### Example 2: Targeting eNOS Uncoupling as a Common Modulator of ECD in LN

Systemic lupus erythematosus (SLE) is a disease in which tissue-targeted immune complexes induce clinical disease through secondary innate immune responses. Endothelial cells (EC) are conditional effector cells that act as biosensors to respond to inflammatory signals by inducing chemotaxis, adhesion, rolling, and diapedesis of inflammatory cells into tissue during acute infection. When chronically stimulated by circulating, tissue-tropic, or tissue-resident factors, endothelial cell dysfunction (ECD) occurs (Mai, J. et al., 2013, J. Hematol. Oncol., 6:61). This phenomenon is seen in SLE to a much greater degree than in health (El-Magadmi, M. et al., 2004, Circulation, 110:399-404). Clinical manifestations traditionally associated with chronic ECD are atherosclerosis and vasculopathy, which are both more prevalent in SLE (Roman, M. J. et al., 2007, Arthritis Rheum., 56:3412-9) and associate with lupus nephritis (LN) (Doria, A. et al., 2003, Ann. Rheum. Dis., 62:1071-7). It is proposed to target ECD as a mechanism of tissue inflammation in LN (Lopes-Virella, M. F. et al., 2003, Front Biosci., 8:s750-68; Jacobi, J. et al., 2005, Free Radic. Biol. Med., 39:1238-48).

Endothelial cells act as a barrier that prevents circulating immune cells from entering tissue. During homeostasis, endothelial nitric oxide synthase (eNOS) modulates the expression of inflammatory molecules. The product of functional eNOS is nitric oxide (NO), which is a membrane-permeable gas that reversibly modifies protein thiols through s-nitrosylation in a fashion similar to phosphorylation. This s-nitrosylation can modify inflammatory transcription factors such as NF-κB, HIF1α, and AP1 to reduce transcriptional activity (Sha, Y. et al., 2012, Biochem. Biophys. Acta, 1820:701-11). Endothelial cells are among the first to be activated by circulating factors through membrane receptors (CD36 and toll-like receptors (TLR) 2 and 4) or endosome receptors (TLRs 3, 7, and 9). This stimulation leads to expression of chemokines/cytokines (IL1β, IL6, IL8, MCP1, RANTES, and TNFα), growth factors (GCSF, GMCSF, PDGF, and VEGF) and adhesion molecules (VCAM1 and ICAM1) that lead to tissue infiltration of immune cells.

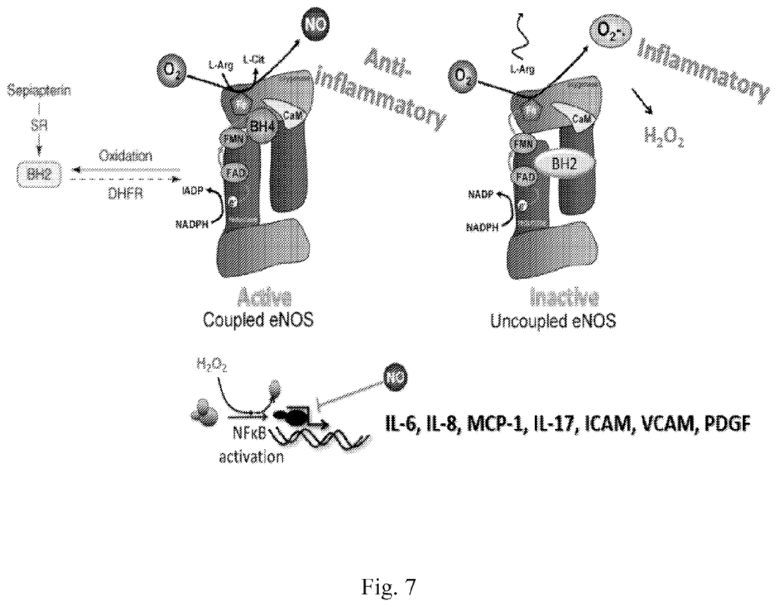

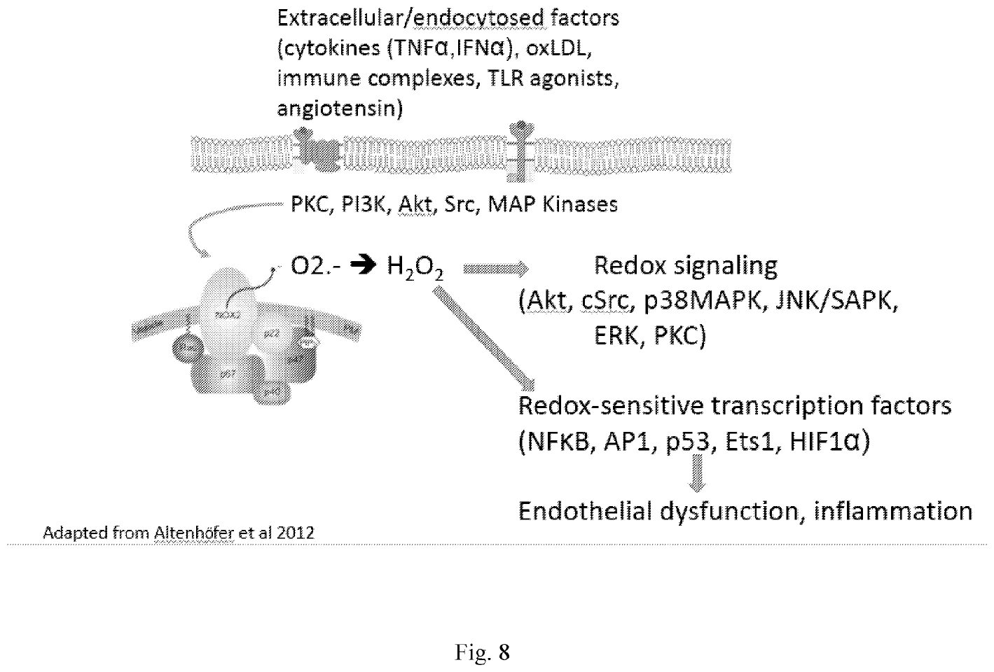

The published (Jones, Buie, J. N. et al., 2019, Medicine, 6:e000294; Buie, J. J. et al., 2017, J. Immunol, 199:1979-88) and preliminary studies suggest that there are multiple mechanisms through which SLE serum induces endothelial dysfunction. Clinically, targeting any one at the membrane receptor may be insufficient to restore endothelial function. However, multiple receptors on endothelial cells stimulate NADPH oxidase-dependent reactive oxygen production (see FIG. 8). Because eNOS is a redox-regulated enzyme, a final common pathway for eNOS dysfunction is oxidation of the cofactor tetrahydrobiopterin (BH₄) to BH₂. Without BH₄, eNOS homodimers uncouple electron transfer from the reductase to the oxygen domain (FIG. 7, adapted from (Kern, M. et al., 2011, PCOM Biomedical Studies Student Scholarship: Philadelphia College of Osteopathic Medicine; Li, J. M. et al., 2004, Am J. Physiol. Regul. Integr. Comp. Physiol., 287:R1014-30)). This uncoupling feeds forward to pro-inflammatory ROS production by eNOS monomers rather than anti-inflammatory NO production by eNOS homodimers (Bendall, J. K. et al., 2014, Antioxidants & redox signaling, 20:3040-77).

Several lines of evidence support mechanisms and manifestations of chronic ECD in SLE. It is previously published that type I interferon response genes can be induced in endothelial cells by lupus serum in vitro. Interferon alpha, when applied to endothelial cells, induced an ECD phenotype with reduced NO production (Buie, J. J. et al., 2017, J. Immunol., 199:1979-88). Furthermore, preliminary data suggest that IgG in lupus serum also contributes to ECD and reduced NO production. These combined data support the notion that multiple circulating factors lead to chronic ECD in SLE. For instance, receptor activation that induces ECD in other models are also prominent in SLE, such as ligation of the type I interferon receptors (Buie, J. J. et al., 2017, J. Immunol., 199:1979-88), FcRγ receptors (Tanigaki, K. et al., 2009, Circ. Res., 104:1275-82), LOX1 (receptor for oxidized LDL) (Liao, J. K. et al., 1995, J. Biol. Chem., 270:319-24; Searles, C. D. et al., 2006, Am. J. Physiol. Cell Physiol., 291:C803-16; Smirnova, I. V. et al., 2004, Am. J. Physiol. Renal, Physiol, 287:F25-32), toll-like receptors (cell surface and intracellular) (Stark, R. J. et al., 2018, J. FASEB, 32:945-56), and the angiotensin II receptor (Oak, J. H. et al., 2007, Diabetes, 56:118-26; Outdot, A. et al., 2006, Free Radic, Biol. Med., 40:2214-22). However, multiple pathways converge on activation of NADPH oxidase (Li, J. M. et al., 2004, Am. J. Physiol. Regul. Integr. Comp. Physiol., 287:R1014-30; Altenhofer, S. et al., 2012, Cell Mol. Life Sci., 69:2327-43), production of reactive oxygen, and subsequent uncoupling of eNOS (El-Magadmi, M. et al., 2004, Circulation, 110:399-404). Intracellular ROS signals through redox-sensitive transcription factors such as NFκB, AP1, Ets1, p53, and HIF1α and kinases such as p38 MAP kinase, ERK, and JNK (Drummond, G. R. et al., 2014, Trends in Endocrinology & Metabolism, 25:452-63) (FIG. 8) to induce synthesis of inflammatory chemokines and adhesion molecules such as IL6, IL8, and MCP1 (Volk, T. et al., 2000, Mol. Cell Biochem., 206:105-12; Miyoshi, T. et al., 2010, Immunology, 131:331-9). NO, on the other hand, modulates this process through nitrosylation of, for instance, NFκB (Grumbach, I. M. et al., 2005, J. Mol. Cell Cardiol., 39:595-603; Wung, B. S. et al., 2001, Arterioscler Thromb Vasc. Biol., 21:1941-7). Relevant to lupus, this laboratory has demonstrated that eNOS modulates MCP1 production in murine lupus LN, which confirms relevance of these pathways to SLE (Gilkeson, G. S. et al., 2013, PLoS One, 8(5)). Thus, targeting downstream pathways from ROS production may reduce the number of pathways to target in SLE. Effective, orally available, antioxidant scavengers are not currently available for human use but are a future potential means for targeting this final common pathway (Forman, H. J. et al., 2015, Free Radic. Biol. Med., 78:233-5). Targeting eNOS uncoupling as a common modulator of ECD is effective in vitro and in vivo in other model systems. It has not been tested in LN as a prototype inflammatory disease in vivo.

In preliminary studies, serum from SLE patients with active proliferative lupus nephritis (LN) selectively induced chemotaxis and adhesion of neutrophils to human glomerular endothelial cells (Jones, Buie, J. N. et al., 2019, Medicine, 6:e000294). This is an example of induction of an inflammatory EC phenotype with factors unique to active more than inactive LN. It is also shown that uncoupling of endothelial NO synthase (eNOS) is a major factor leading to endothelial ECD induced by lupus serum. It is recently published that ECD induced by lupus serum can be reversed by targeting eNOS uncoupling in vitro with sepiapterin (Jones, Buie, J. N. et al., 2019, Medicine, 6:e000294).

Sepiapterin is a viable candidate for in vivo targeting of ECD. Sepiapterin is transported into cells and is resistant to oxidation; whereas BH₄ is rapidly oxidized and has variable effectiveness in treating chronic ECD in vivo (Bendall, J. K. et al., 2014, Antioxidants & Redox Signaling, 20:3040-777; Ohashi, A. et al., 2011, Mol. Genet. Metab., 102:18-28). Once in EC, sepiapterin is metabolized through the salvage pathway to active BH₄ via sepiapterin reductase (SR) and dihydrofolate reductase (FIG. 7). It increases intracellular BH₄ levels more effectively than exogenous BH₄ (Crabtree, M. J. et al., 2011, Nitric Oxide: Biol. and Chemis., 25:81-8). There is a large literature demonstrating successful improvement of vascular function using sepiapterin in diseases such as hypertension and diabetes (Pannirselvam, M. et al., 2003, Br. J. Pharmacol., 140:701-6; Keller, A. C. et al., 2018, Oxid. Med. Cell Longev., 2018:7363485; Schmidt, T. S. et al., 2010, Clin. Sci., 119:131-42). The safety profile in humans with phenylketonuria and tetrahydrobiopterin deficiencies is favorable (Smith, N. et al., 2019, Mol. Genet. Metab., 126(4):406-12).

Taken together, these findings strongly suggest that targeting uncoupling of eNOS with sepiapterin is a novel approach to targeting ECD in SLE to address tissue-targeted chronic inflammation.

Several decades ago, Hashimoto et al. demonstrated that neutrophils treated with serum from patients with active lupus adhered to endothelial cells in greater numbers, had more aggregation, and produced increased reactive oxygen (Hashimoto, Y. et al., 1982, Arthritis Rheum., 25:1409-18). This was an important study, as it elevated the role of neutrophils as pathogenic mediators and lupus. However, while SLE is a systemic disease, it often selectively affects organs at sites of immune complex deposition/formation. Glomerular endothelial cells are activated by LN serum, and a cytokine mix can activate STAT1, STAT2, and NFκB (Dimou et al., 2019, Sci Rep, 9(1):8348). Transcriptional regulation in response to SLE serum was not described, which leaves a gap in the literature. Because redox signaling mechanisms were not explored, another gap remains. Therefore, to understand why neutrophils migrate and adhere to vascular endothelium, it is rational to investigate the process through which local ECs (in this case glomerular endothelial cells) are activated at sites of inflammation. This represents a paradigm shift in the treatment of inflammatory disease through targeting pathogenic redox signaling in EC as effector cells. This is the first study to compare directly gene expression and phosphorylation pathways in endothelial cells induced by serum from patients with active versus inactive LN. These studies enhance our understanding of organ-specific endothelial activation in LN. Finally, this is the first evaluation of pathways leading to and resulting from redox signaling in EC induced by active LN serum. The findings of these studies inform and provide the rationale for studies that focus on therapeutic targeting of endothelial dysfunction and chronic inflammation in human LN.

### Veteran Health Relevance

The results of these studies contribute to the knowledge of how to target ECD in LN as a therapy. It enhances understanding of mechanisms behind pathogenic redox signaling in EC so that it may more effectively be targeted. Women, particularly African-American women, are affected disproportionately by SLE and are a growing demographic in the armed services. Therefore, effective therapy with low toxicity could reduce disparities in SLE. Discoveries from this project can provide the rationale for novel therapy of high-impact diseases affecting veterans such as hypertension, diabetes, and atherosclerosis (Bendall, J. K. et al., 2014, Antioxidants & redox signaling, 20:3040-77; Schmidt, T. S. et al., 2010, Clin. Sci., 119:131-42).

Lupus Nephritis Serum Induces an Adhesion and Migration EC Phenotype

It is published that lupus serum induces endothelial cell dysfunction (adhesion and migration of neutrophils and reduced NO production) in human umbilical vein endothelial cells (Jones Buie, J. N. et al., 2019, Medicine, 6:e000294). To provide relevance to lupus nephritis, additional experiments has been conducted in human renal glomerular endothelial cells (HRGECs). To determine if circulating factors that induce endothelial dysfunction are unique to patients with nephritis, serum samples from patients with and without nephritis were studied. Because it was observed that, even in controls, hypertensive serum induced endothelial dysfunction by these assays, lupus and control patients with and without hypertension were studied. Finally, to determine if disease activity resulted in changes in endothelial function, individual lupus patients were studied longitudinally during periods of disease inactivity and disease activity (SLE Disease Activity Index scores differing by more than 4 points). Active lupus nephritis was defined as a flare with a rise in urine protein/creatinine greater than 0.5 g/g that required a change in immunosuppressant therapy. In both the adhesion and migration experiments, sera from lupus and control patients were incubated with HRGECs in the third or fourth passage. For the adhesion experiments, washed cells previously treated with serum were co-cultured with Calcein AM-labeled neutrophils from normal, healthy controls. Adhesion of neutrophils was quantified by fluorescence intensity after removal of non-adherent cells. For the migration experiments, washed cells previously treated with serum were cultured in medium that was then collected and placed into the lower chamber of a transwell apparatus. Labeled neutrophils were placed in the upper chamber, and migration to the lower chamber was quantified by fluorescence intensity.

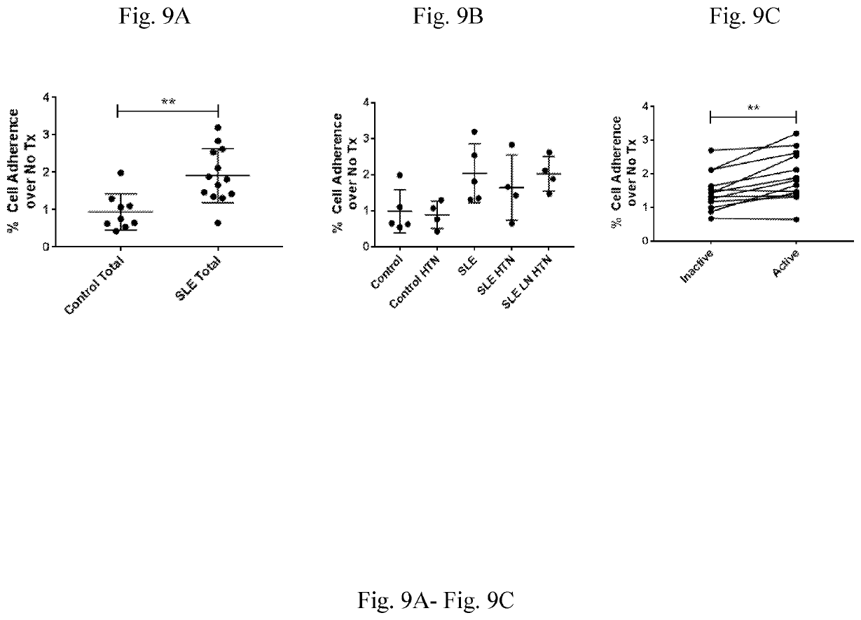

Lupus serum induced greater adhesion than control serum (FIG. 9A). Serum from all lupus phenotypes (LN and HTN) induced the most neutrophil adhesion (FIG. 9B). Serum from SLE patients during active disease induced greater adhesion than serum of the same patients during inactive disease (FIG. 9C). This suggests that circulating factors that induce neutrophil adhesion are increased during disease activity and provide some insight into how ECD-mediated local inflammation occurs during disease activity.

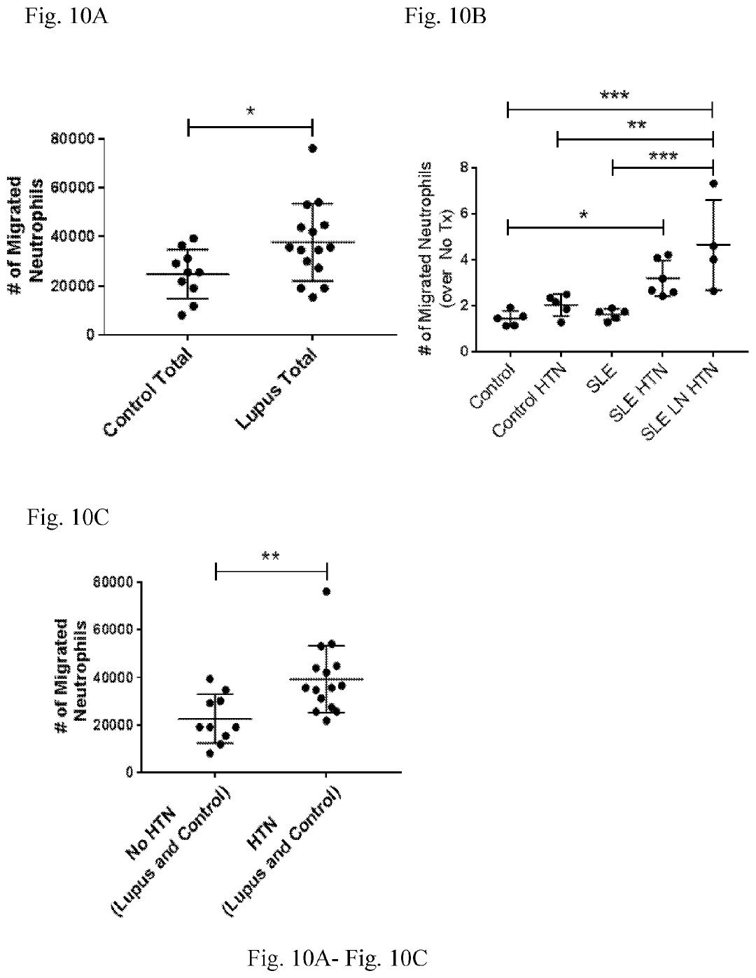

In the migration experiments, HRGECs treated with SLE serum induced more neutrophil chemotaxis than control serum (FIG. 10A). Chemotaxis to HRGECs treated with serum from patients with lupus and hypertension and patients with LN was significantly greater than that from controls or lupus patients without hypertension (FIG. 10B). Of interest, hypertension itself, whether in patients with lupus or without lupus, associated significantly with a migration endothelial cell phenotype (FIG. 10C). This suggests that there may be common mechanisms leading to chemotaxis in LN and hypertension and provides the rationale for using sepiapterin, which is effective in treating the angiotensin model of hypertension, as a prototype agent for targeting endothelial dysfunction in LN.

### Active LN Serum Induces PDGF-BB Production in HRGECs.

To explore potential mechanisms through which endothelial cell activation induces migration of neutrophils to endothelium that is selective to lupus nephritis, conditioned medium from the trans-well experiments for multiple chemokines, cytokines, and growth factors (including GM-CSF, CXCL1 (GROα), CCL2 (MCP1), CXCL8 (IL8), CCLS (RANTES), IL6, and platelet-derived growth factor (PDGF-BB)) were examined in the same sample by the Luminex bead array. The only chemotactic factor that increased with active lupus nephritis was PDGF-BB. While PDGF-BB is labeled as a growth factor, it is a chemokine for neutrophils with similar potency to that of complement split product C5a (Deuel, T. F. et al., 1982, The J. of Clin. Inv., 69:1046-9). This suggests that, as in other model systems, LN serum induces signaling in HRGEC that leads to changes in expression of chemokines, cytokines, and growth factors. Levels of factors in individual samples were correlated with each other across lupus and control samples. Several of the factors correlated highly with each other (p<0.05, M-CSF, CXCL1, MCP3, PDGF BB, IL-15, IL6, CXC8, CCL 2, and CCCL5). This correlation suggests common signaling pathways. Of note, transcription of each of these is regulated by either redox-sensitive transcription factors or kinases (FIG. 8). Taken together, these data support the notion that targeting redox signaling may be effective in reducing chronic ECD in LN.

LN Serum Induces eNOS Uncoupling

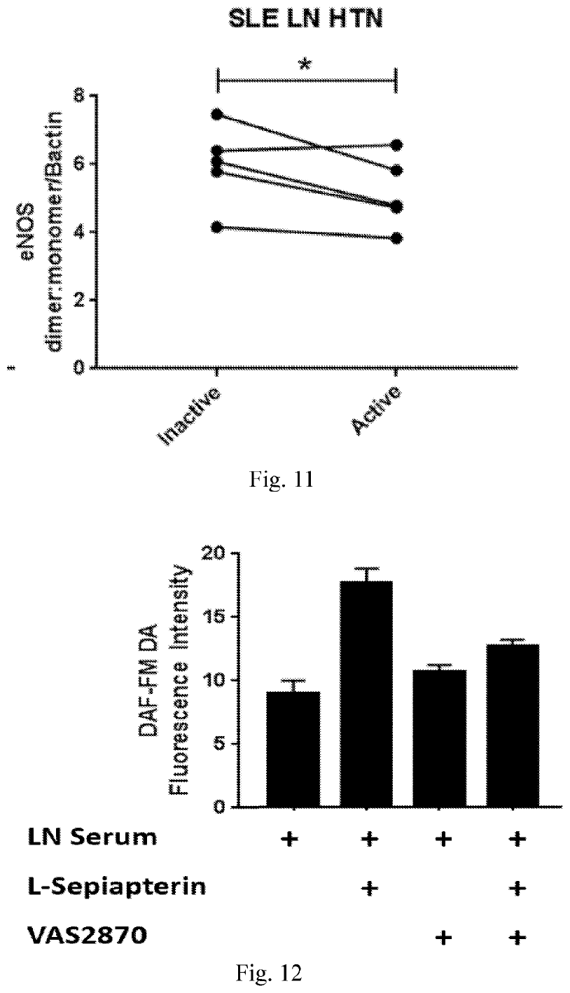

To determine the effect of LN serum on eNOS uncoupling, protein from cells treated as in FIG. 10 was separated on a non-denaturing gel, transferred to a blot, and immune-stained for eNOS. The ratio of dimers to monomers was reported. For only cells treated with LN serum, the ratio of dimers to monomers reduced during flares of LN (FIG. 11).

NO Production by HRGEC Treated with LN Serum is Restored with Sepiapterin

The previously published study (demonstrating that sepiapterin improved EC NO production induced by SLE serum) was performed on HUVEC. The inflammatory phenotype of HUVECs differs from microvascular endothelial cells and may not reflect mechanisms in glomerular endothelial cells. To confirm the uncoupling effects lead to functional changes in uncoupling and NO production by EC treated with LN serum in HRGEC, HRGEC as in FIG. 10 was treated with LN serum in a black walled 96-well plate. Sepiapterin was added to culture alone or in combination with VAS2870, a validated NADPH oxidase specific inhibitor (Altenhofer, S. et al., 2012, Cell Mol. Life Sci. 69:2327-43). VAS2870 alone was added to culture to determine if reducing superoxide production from NADPH oxidase alone was sufficient to prevent uncoupling of eNOS. NO production was quantified using DAF-FM fluorescence with urate added to prevent ONOO— fluorescence. LN serum-treated HRGEC NO production was increased more with sepiapterin than VAS2870 or VAS270+sepiapterin (FIG. 12). These results suggest that sepiapterin is superior in restoring coupling of eNOS. It also suggests that VAS2870 alone is not effective in restoring endothelial function/and NO production. While not wishing to be bound to any particular theory, one possible explanation for this finding is that uncoupled NOS is a significant source of ROS in LN (Njoku, C. J. et al., 2005, J. Investig. Med., 53:347-52).

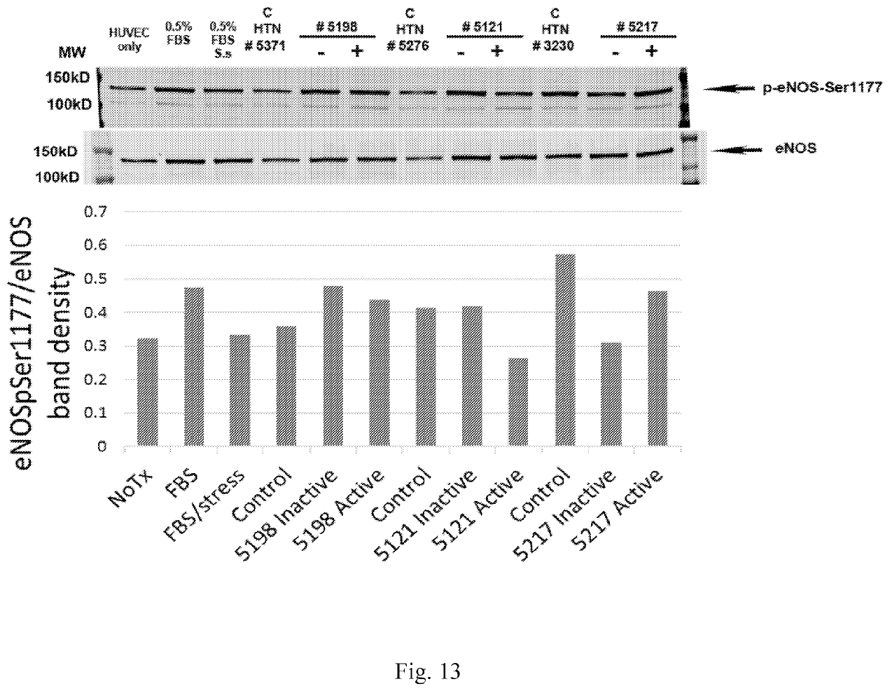

Endothelial NOS activity is regulated through a variety of mechanisms. Phosphorylation at Ser1177 increases activity. To address the effect of lupus serum on eNOS phosphorylation, Western blots were performed on cell lysates from cells treated as in FIG. 9 and FIG. 10. Blots were immune-stained for eNOS-pSer1177, stripped, and re-probed for eNOS. The ratio of eNOS-pSer1177/eNOS was reported. A consistent trend of reduced phosphorylation of eNOS at Ser1177 with lupus versus control lysates was not observed. In fact, some hypertensive patients have quite low levels as well (FIG. 13). Therefore, the current work focuses on targeting uncoupling of eNOS for therapy. These studies demonstrate the ability to study EC NO and ROS production with inhibitors/agonists in a high-throughput format.

Sepiapterin (SPR) and Dihydrofolate Reductase (DHFR) are Expressed in HRGEC Treated with LN Serum

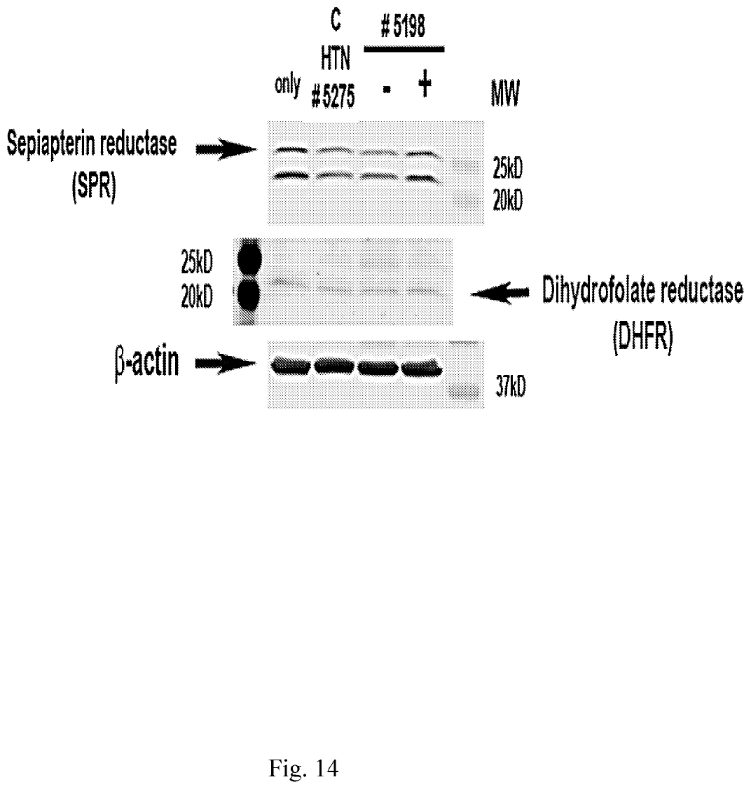

For sepiapterin to be effective, it must be transported into cells and metabolized through the salvage pathway to BH₄ by SPR and DHFR (FIG. 7). To demonstrate that these enzymes are present in HRGECs under LN conditions, cells were cultured as in FIG. 10 with LN serum and Western blot were performed on lysates from cells for SPR and DHFR. The amount of SPR and DHR did not differ between cells treated with control or LN (subject 5198) active or inactive serum (FIG. 14).

### Example 3: Effect of Therapy to Recouple eNOS on Renal Inflammatory Infiltrates, Endothelial Function, and Pharmacodynamic Markers of Response in an Acute Model of Immune Complex-Mediated Nephritis

These studies demonstrate that therapy known to restore endothelial homeostasis in vascular diseases reduces inflammatory infiltrates in the kidney downstream of immune complex formation. Vascular function and biomarkers of EC inflammatory activation that are redox regulated change significantly with therapy. Because LN is a heterogeneous disease, the effect of targeted therapy on inflammatory infiltrates downstream of immune complex deposition is studied. The anti-glomerular basement membrane (GBM) model of immune complex-mediated nephritis is ideal for such studies, as it removes variability in immune complex deposition and avidity seen in spontaneous models (Du, Y. et al., 2008, Arch. Immunol. Ther. Exp. (Warsz), 56:31-40). The resulting effects on biomarkers and vascular function is a reflection of secondary innate and cellular responses. Of interest, NFκB, a redox-regulated transcription factor, is highly expressed in this model, making it relevant to the study of redox-targeted therapy (Kim, J. H. et al., 2004, Kidney Int., 66:1826-37).

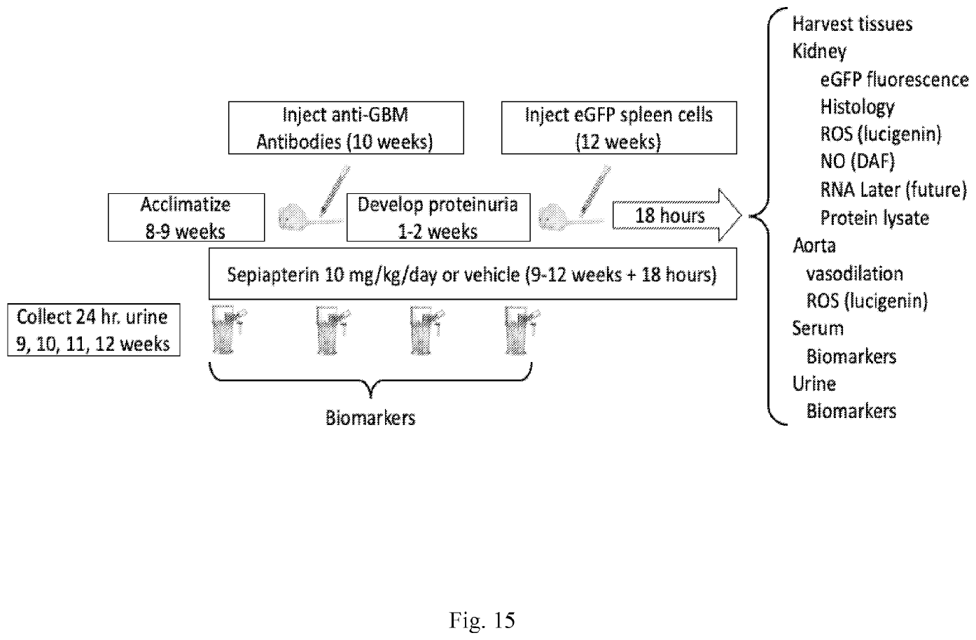

The anti-glomerular basement membrane (GBM) model is used as a prototypic immune complex nephritis ideal for the study of secondary innate and cellular immune responses (Du, Y. et al., 2008, Arch. Immunol. Ther. Exp. (Warsz), 56:31-40). For these studies, thirty mice are divided into two groups. The first group is treated with vehicle, while the second is treated with sepiapterin (10 mg/kg/day orally in powdered chow) for one week before inducing nephritis with injection of anti-GBM antibodies as described (Xie, C. et al., 2004, The J. of Immunology, 172:5047). The start date for anti-GBM injection is staggered to accommodate 10 mice per week given the rate-limiting steps of performing the aortic ring and tissue harvest experiments. Nephritis onset occurs between 7 and 14 days after injection, so treatment continues for 14 days and then enhanced green fluorescent protein (eGFP) labeled spleen cells are injected into the tail vein of all mice. After 18 hours, renal tissue is harvested for study of endpoints. Aortas are harvested to study vascular function ex vivo. Urine is collected in metabolic cages at 0, 1, and 2 weeks after anti-GBM treatment for the study of urine biomarkers of response. Serum is collected at euthanasia for the study of serum biomarkers of response and systemic oxidant stress in response to therapy (FIG. 15).

Induction of Anti-GBM Nephritis

Immune complex-mediated nephritis was chosen as a prototype for inflammation associated with endothelial cell dysfunction. Others have shown that anti-GBM nephritis is exacerbated when eNOS is genetically inactivated (Heeringa, P. et al., 2000, Am. J. Pathol., 156:879-88). Therefore, this model studies the restoration of eNOS function with sepiapterin. 129 mice have robust downstream inflammatory responses to anti-GBM antibodies when compared to other strains (Xie, C. et al., 2004, The J. of Immunology, 172:5047). Therefore, 129 mice at 10 weeks of age are used and injected with 200 μg of anti-GBM nephrotoxic antibodies into the tail vein. Because clinical glomerulonephritis progresses between 7 and 14 days in this model, the primary endpoint is studied at 14 weeks after induction with anti-GBM toxic serum. If significant proteinuria (>100 mg/dL by dipstick, does not occur in at least 75% of mice in any group by 14 days, the experiment is extended to achieve that endpoint.

Primary Endpoint—Renal Cortical Cellular Infiltration

It is examined whether targeting ECD by recoupling eNOS with sepiapterin reduces inflammatory infiltrates in cortical tissue in immune complex glomerulonephritis. Cellular infiltrate into the kidney is determined by the fluorescence intensity of eGFP cells infiltrating the cortex. To accomplish this, eGFP mice is generated. Spleen cells from 10-14 week-old eGFP mice are isolated as described (Jadeja, R. N. et al., 2015, J. Vis. Exp., e50997), or alternatively from Jackson Laboratories (haplotype b). GBM-treated mice (haplotype b) are injected by tail vein with 2×10⁶ spleen cells from eGFP mice. 20×10⁶ million spleen cells are isolated from one eGFP mouse. After 18 hours, mice are euthanized, and harvested kidney sections are analyzed for interstitial and glomerular fluorescence intensity as described (Sato, S. et al., 2014, Clin. Exp. Immunol., 177:102-9). Three-micron sections of formalin-fixed cortical tissue are stained with H&E and PAS as described (Sundararaj, K. et al., 2018, Am. J. Physiol. Renal Physiol, 314:F630-F42). The extent of glomerular and interstitial pathology is graded using the revised ISN/RPS activity and chronicity scores (Bajema, I. M. et al., 2018, Kidney Int., 93:789-96). Cortical sections are immuno-stained for IgG and C3, and fluorescence intensity are determined by using semi-quantitative methods.

Ex Vivo Vascular Function

It is examined whether sepiapterin improves eNOS-dependent endothelial function as measured by endothelium-dependent vasodilation of the aorta ex vivo. Immune-complex nephritis induces endothelial dysfunction as is seen in human SLE. Because LN is an independent risk factor for atherosclerosis in SLE patients, restoration of endothelial function may also reduce the burden of cardiovascular disease in this at-risk population. In addition, results of experiments described above under primary endpoint-renal cortical cellular infiltration can be interpreted only in light of the extent to which ECD is reduced in individual mice treated with sepiapterin as a pharmacodynamic marker of adequate dosing. Therefore, endothelium-dependent vascular smooth muscle relaxation is determined at the time of euthanasia, after the onset of clinical nephritis. The extent of restoration of endothelial dysfunction is correlated with the extent of cellular influx into glomeruli and the interstitial spaces.

These experiments are performed as described (Wheeler, J. B. et al., 2015, J. Am. Heart Assoc., 4:e001744; Jadeja, R. N. et al., 2015, J. Vis. Exp., e50997; Akerman, A. W. et al., 2019, J. Am. Heart Assoc., 8:e010332; Ruddy, J. M. et al., 2010, J. Surg. Res., 160:333-9; Ruddy, J. M. et al., 2009, Circulation, 120:S262-8; Keller, A. C. et al., 2018, Oxid. Med. Cell Longev., 2018:7363485). The descending thoracic aorta from mice in above described methods is harvested and immediately placed in cold Krebs-Hanseleit buffer. The aorta is divided into 3 mm long segments (typically 2 segments per mouse) which is mounted on parallel wires in a water jacketed tissue myograph system (Radnoti, Monrovia, Calif.; 25 ml), maintained at 37° C. and connected to an isometric force transducer (Radnoti, Monrovia, Calif.). The vessel segments is then equilibrated for 30 minutes, in the absence of tension, and washed every 15 minutes with warm Krebs-Hanseleit solution aerated with 95% O₂/5% CO₂, and supplemented with EGTA (5 mM) to minimize calcium-induced smooth muscle cell contraction. Optimal tension is determined as previously described using aortas from control animals without GBM nephritis (Jadeja, R. N. et al., 2015, J. Vis. Exp., e50997; Akerman, A. W. et al., 2019, J. Am. Heart Assoc., 8:e010332; Ruddy, J. M. et al., 2010, J. Surg. Res., 160:333-9; Ruddy, J. M. et al., 2009, Circulation, 120:S262-8).

Briefly, stress relaxation of the aortic segments is quantified by sequentially stretching vessel segments in 0.1 g increments (0.2 g-1.2 g of applied tension) and measuring residual tension after 3 minutes to calculate percent relaxation. During the equilibration period, the ECM microfibrils adapts and relaxes, demonstrating a decay in the transduced vessel tension. When the difference between the initial applied tension and the value recorded at the end of the equilibration period is significantly less than 10%, the applied tension value was equivalent to the “passive tension” of the vessel. Mean percent decline at each level of applied tension is compared to a standard of 10% using a one-sample t-test. Measurements are recorded using Biobench software (National Instruments, Austin, Tex.).

In similar fashion, to determine the contractile properties of the aorta, rings harvested from mice is equilibrated at 0.4 g of tension for 30 minutes in standard Krebs-Hanseleit buffer without EGTA. Contraction is stimulated with the addition of KCl to a final concentration of 100 mM in the tissue bath, and the peak contractile force (g) generated over the ensuing 8 minutes is recorded. The rings are then washed with Krebs-Hanseleit solution to remove KCl. These steps are then repeated as above from 0.1 g-1.2 g of applied tension as previously described. The optimal tension of the aortic ring is an equilibration point where all passive tension has been overcome and maximum active (contractile) tension can be generated by the medial smooth muscle cells. Aortic contractility and relaxation is measured in control and GBM mice, treated without and with sepiapterin. Aortic rings is equilibrated at optimal tension for 30 minutes in Krebs-Hanseleit buffer (without EGTA). The rings are then induced to contract using increasing concentrations of phenylephrine (PE) (from 10⁻¹⁰ M to 10⁻⁵ M). The endothelium-derived relaxation is investigated by precontracting the aortic rings to 70% of the maximum contraction with PE, and then inducing relaxation with increasing concentrations of acetylcholine (Ach) (from 10⁻⁹ M to 10⁻⁶ M). Endothelium-independent relaxation is tested by contracting the aortic rings to 70% of the maximum contraction with PE, and then relaxing the rings with increasing concentrations of sodium nitroprusside (SNP) (10⁻⁸M to 10⁻⁵ M). Percent relaxations to Ach and SNP is calculated, respectively, by: 1) ΔForce_{[Ach]}/ΔForce_{[70%} contraction]×100, and 2) ΔForce_{[SNP]}/ΔForce_{[70%} contraction]×100.

A second set of studies examine the ability of sepiapterin to improve endothelium-dependent vasorelaxation with added sepiapterin in vitro. In these studies, the aortic rings from mice is precontracted to 70% of the maximal contraction with PE, and relaxation is induced with either increasing concentrations of a nonspecific NOS-inhibitor N-Nitro-l-arginine-methylester (L-NAME, 100 μM) or with sepiapterin (5 μM). Percent relaxations to L-NAME and sepiapterin is calculated, respectively, by: 1) ΔForce_{[L-NAME]}/ΔForce_{[70%} contraction]×100, and 2) ΔForce_{[sepiapterin]}/ΔForce_{[70%} contraction]×100. These endpoint studies are essential and serve as a pharmacodynamic marker of the effectiveness of in vivo treatment with sepiapterin relative to the maximal in vitro response in the second experiments. The results of these experiments inform future work on using sepiapterin to improve vascular function and prevent cardiovascular disease in patients with lupus, as well as higher impact diseases like chronic kidney disease, diabetes, and hypertension. This study is the first description of eNOS-targeted therapy to improve endothelial function in immune complex-mediated nephritis.

Biomarkers

Serum and Urine Biomarkers of Redox-Regulated Renal Inflammation

It is examined whether Redox regulated EC-derived factors induced by LN serum are reduced with sepiapterin therapy. The preliminary studies indicate that LN serum induces the production of PDGF-BB by EC. Furthermore, the studies of eNOS−/− mice on a lupus background indicate that eNOS modulates MCP1 (CCL2) production. In previously published work, several urine biomarkers serve to indicate the response to therapy in human LN, specifically osteoprotegerin (OPG), interleukin 8 (IL8), monocyte chemotactic protein (MCP1/CCL2), and tumor necrosis factor-like weak inducer of apoptosis (TWEAK/Fn14), all of which can be expressed in endothelial cells (Lai, K. N. et al., 1997, Scand. J. Rheumatol., 26:461-7; Donohue, P. J. et al., 2003, Arteroscler Thromb. Vasc. Biol., 23:594-600; Cross, S. S. et al., 2006, Int. J. Cancer, 118:1901-8; Lou, N. et al., 2017, Mol. Immunol., 81:59-66). Of interest, the expression of many of these factors is regulated by NFκB, itself a redox-regulated transcription factor (Kabe, Y. et al., 2005, Antioxid. Redox Signal, 7:395-403). Therefore, the levels of biomarkers above in the urine (before and after sepiapterin therapy and induction of GBM nephritis) and serum (at the time of euthanasia) are studied by Luminex bead array as described (Wolf, B. J. et al., 2016, Arthritis & Rheumatology, 68:1955-63; Taylor, T. P. et al., 2012, Biomark. Insights, 7:1-8) using the MUSC Immune Monitoring and Discovery Core as in the preliminary studies. Biomarkers that are significantly different between groups are analyzed by ELISA in the cortical lysates as a confirmation of renal source.

Biomarkers of Systemic Oxidative Stress

Additional biomarkers of systemic oxidative stress are studied in the urine and serum. Specifically, indirect systemic biomarkers of oxidative stress are chosen that are readily measurable with commercial assays to accelerate the translation to human studies. Malondialdehyde is an oxidative modification of proteins that can be measured by ELISA. 8-OHdG is an oxidative modification of DNA that reflects mitochondrial ROS production that is excreted in the urine. Both oxidation products are increased in SLE (Shah, D> et al., 2014, Biomed. Sci., 21:23; Olivares, D. et al., 2018, Clin. Sci., 132:569-79; Kurien, B. T. et al., 2003, Life Sci. 73:1655-66). Therefore, urine and serum levels of these systemic markers of ROS production in both groups using commercial assays (Sigma lipid peroxidation (MDA) and R&D Systems 8-oxo-dG ELISA kits) are compared. To validate them as systemic markers of renal and vascular SO production in the setting of endothelium-dependent inflammation, these markers are correlated with GFP cellular infiltration and glomerular scores, urine and serum biomarkers, renal cortical and aorta superoxide production, and aortic ring endothelium-dependent relaxation as discussed above.

Lucigenin Assay to Detect Renal and Aorta Superoxide Production

Superoxide production from renal cortical and aorta lysates from snap frozen tissue is measured as described (Oates, J. C. et al., 2013, Lupus, 22:1361-70). This assay detects spontaneous superoxide production through chemiluminescence. The following controls are used to determine the enzyme source of superoxide: No treatment, treatment with L-NAME to inhibit uncoupled pan-NOS production, L-NIL (10 μM) to inhibit uncoupled eNOS-specific ROS production, DPI (10 μM) and VAS2870 (25 μM) to inhibit NADPH oxidase production, allopurinol (50 μM) to inhibit xanthine oxidase production, and rotenone (20 μM) to reduce mitochondrial nitric oxide production. A positive control consists of added xanthine and xanthine oxidase (Oates, J. C. et al., 2013, Lupus, 22:1361-70). The results of this assay is correlated with the systemic biomarkers as a validation of the commercial assays. In addition, they are correlated with the urine and serum biomarkers to inform possible redox regulation of biomarker expression. Redox regulation of gene expression are addressed in vitro and described below.

The results of these studies provide minimally invasive systemic markers that reflect the in vivo inflammatory renal cortical infiltration and endothelium-dependent vascular function. These results provide the rationale for human studies of these as pharmacodynamic markers of response to therapy.

### Example 4: Effects of Therapy to Recouple eNOS on Renal Inflammatory Infiltrates, Endothelial Function, Pharmacodynamic Markers of Response, and Clinical and Histologic Indicators of LN

A validated model (NZM2410 mouse model) for anti-double-stranded DNA antibody positive immune complex-mediated proliferative LN and SLE is used for experiments to determine the effects of therapy to recouple eNOS on renal inflammatory infiltrates, endothelial function, pharmacodynamic markers of response, and clinical and histologic indicators of LN. This model also has the type I interferon signature (Der, E. et al., 2019, Nat Immunol., 20(7):915-27) seen in human SLE (AMP project cortical biopsy single cell RNA seq (Der, E. et al., 2019, Nat Immunol., 20(7):915-27)) that was demonstrated to be sufficient to induce ECD in vitro (Buie, J. J. et al., 2017, J. Immunol., 199:1979-88). The parent strain, (New Zealand Black×New Zealand White)_{F1} mice, have endothelial dysfunction as in human SLE (Thacker, S. G. et al., 2010, Lupus, 19-288-99)(67). The mice used for this study is taken from an ongoing VA colony; or alternately, are purchased from Jackson (stock number 002676 from Cryo Recovery). Using this colony with the water supply and clean facility, about half of these mice develop proteinuria by 22 weeks, and half die at about 25 weeks untreated. Each of three groups are divided into 15 mice each to compare groups with (n=15) and without (n=15) sepiapterin treatment. Its effect is then determined on three different endpoints in these groups. 1) prevention/prolonging LN onset with sepiapterin monotherapy, 2) in mycophenolate-treated mice, improving/hastening induction of remission with sepiapterin as an adjunctive therapy, and 3) after inducing remission, prolonging/preventing LN flare with sepiapterin monotherapy after mycophenolate withdrawal.

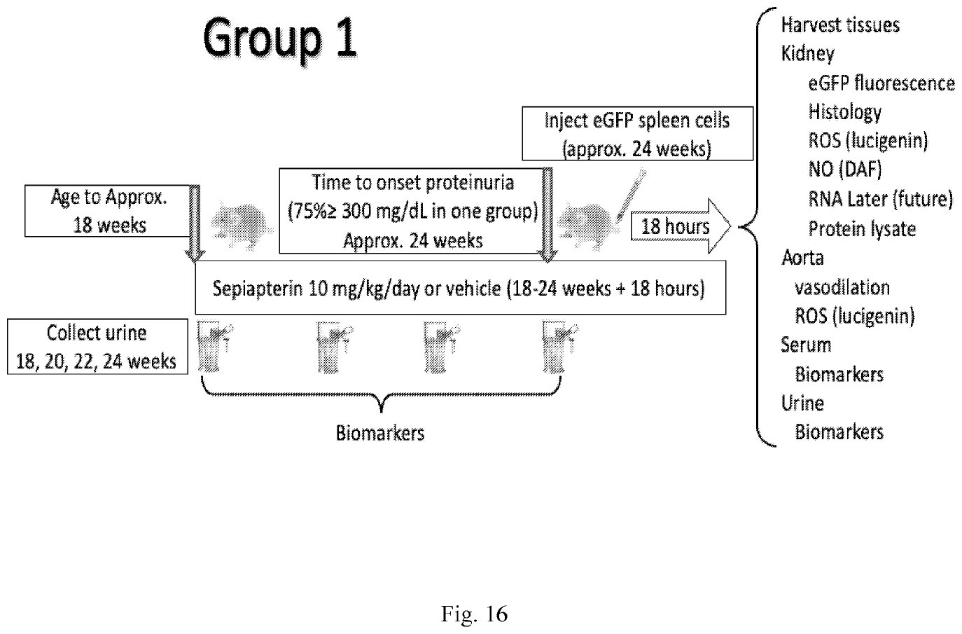

Group 1 (FIG. 16)

The clinical scenario this group is intended to mimic is prevention or delay of nephritis in high-risk individuals before the onset of clinical nephritis. Therefore, NZM2410 mice are monitored weekly by urine dipstick for proteinuria after 12 weeks of age. When at least one mouse develops dipstick proteinuria that is trace or greater, mice are divided into two groups of 15 mice each. One group receives 10 mg/kilogram/day sepiapterin in powdered chow, while the other group receives vehicle in powdered chow. Mice are monitored weekly for dipstick proteinuria. Time to the onset of proteinuria≥300 mg/dL is an additional clinical endpoint. When at least 75% of mice develop dipstick proteinuria≥300 mg/dL in one group, mice are injected with eGFP spleen cells. Eighteen hours later, endpoints are measured as Example 3. Mice meeting clinical endpoints of weight loss, hunched back, or ruffled fur receive eGFP injections early and euthanized for endpoints as in Example 3. 24-hour urine collections are performed at baseline and every two weeks for biomarker analysis.

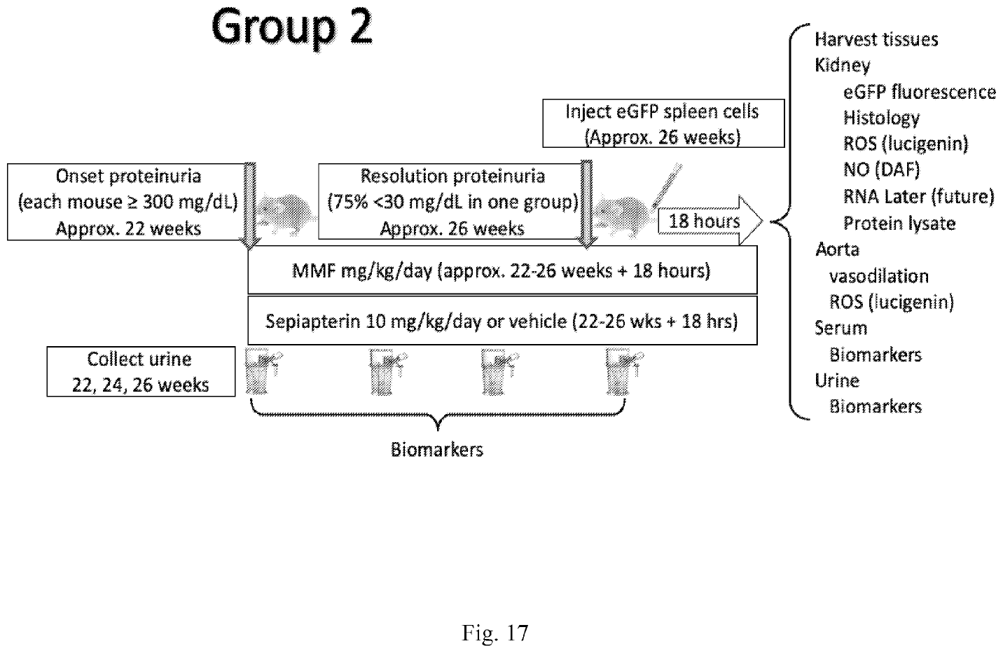

Group 2 (FIG. 17)

The clinical scenario this group is intended to mimic is the use of sepiapterin as adjuvant therapy to standard-of-care mycophenolate mofetil (MMF, 100 mg/kg/day) induction therapy. Individual mice are treated with MMF as clinically, when proteinuria develops at ≥300 mg/dL on two occasions at least two days apart (68). At this time, individual mice are randomized into either sepiapterin treatment (10 mg/kg/day in powdered chow, n=15) or vehicle (n=15) adjuvant treatment. These treatments continue until tissue harvest. At the time that at least 75% of mice in one group resolve proteinuria (≤30 mg/dL), mice are injected with eGFP spleen cells, and tissues are harvested 18 hours later as per Example 3. The number of mice achieving resolution of proteinuria and time to resolution of proteinuria from the start of therapy are clinical endpoints. 24-hour urine collections are performed at baseline and every 2 weeks for the biomarker analyses.

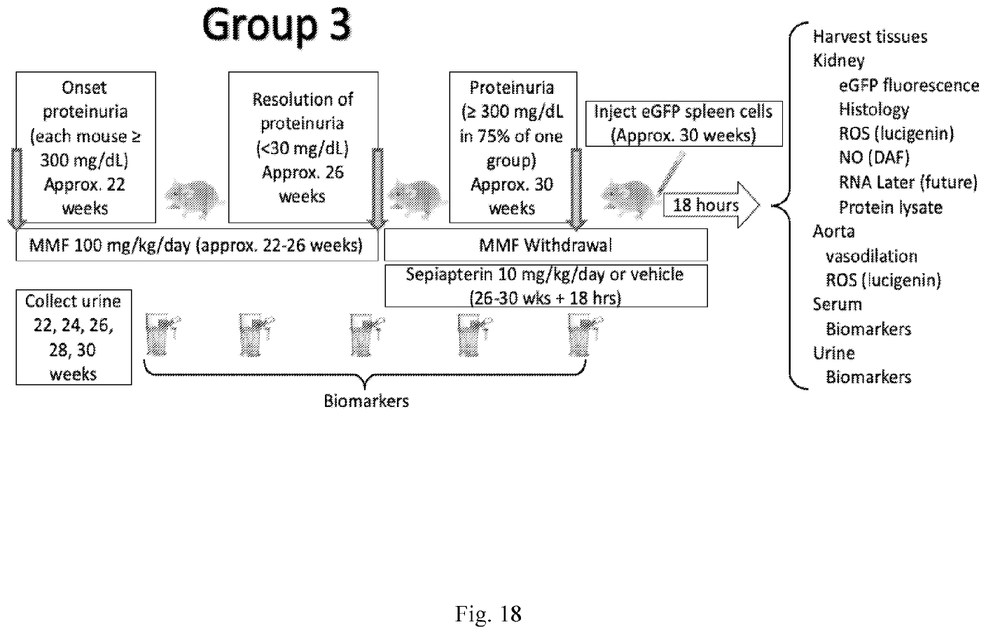

Group 3 (FIG. 18)

The clinical scenario this group is intended to mimic is the prevention of recurrent flare with sepiapterin monotherapy after successful MMF induction therapy. The beginning of this experiment is staggered in anticipation of starting 10 mice every week. Mice are aged until they develop≥300 mg/dL proteinuria by dipstick on two or more occasions at least two days apart. Mice are treated with MMF 100 mg/kilogram/day until resolution of proteinuria to less than 30 mg/dL. As individual mice achieve remission for this endpoint, MMF are withdrawn, and they are randomized alternately to receive either sepiapterin (10 mg/kilogram/day, n=15) or vehicle (n=15). This treatment continues until at least 75% of mice in one group develop proteinuria>300 mg/dL by dipstick on two occasions at least two days apart. When this endpoint is achieved, mice are injected with eGFP spleen cells, and tissues harvested 18 hours later as per Example 3. Two clinical endpoints are measured): 1) time to the onset of proteinuria after MMF withdrawal and 2) number of mice reaching this endpoint when at least 75% of mice in one group achieve it. Mice meeting clinical endpoints of weight loss, hunched back, or ruffled fur receive eGFP injections early and euthanized for endpoints as in Example 3. 24-hour urine collections are performed at baseline and every 2 weeks until the endpoint for the biomarker analyses.

Analysis Plan

The primary outcome for the experiments above is measured via fluorescence intensity of infiltrated cells. Differences between the groups are evaluated using a two-sample t-test or a Mann-Whitney U test as appropriate. Assuming that a 50% reduction from 70 to 35 would be clinical meaningful, 13 animals per group provides 80% power to detect a decrease from 70 to 35 using a 2-sided test at significance p=0.05. Preliminary studies suggest that vehicle treated mice with have mean fluorescence of 70±37 compare to treated mice with have mean fluorescence of 25±18 (Sato, S. et al., 2014, Clin. Exp. Immunol., 177:102-9), indicating the sufficiently for the studies. Time to occurrence of flare is a secondary outcome for groups 1 and 3 and time to resolution of proteinuria in group 2. Difference in time to flare or resolution is evaluated using a log-rank test approach. Thirteen animals per group provides 80% power to detect a 62.5% difference in the proportion of animals that experience either endpoint. Two is added in each group to account for disease-related deaths.

### Example 5: LN-Serum-Induced Mechanisms Leading to NADPH Oxidase-ROS Production and eNOS Dysfunction in Glomerular Endothelial Cells

It is examined whether membrane receptors activated by the heterogeneity of ligands in active lupus nephritis serum induce redox-regulated pathways to inflammatory endothelial cell dysfunction. Knowledge of pathways differentially regulated by active lupus nephritis serum that are modulated by targeting redox-regulated pathways is important for rational development of therapies to target ECD in heterogeneous diseases such as LN.

General Protocol

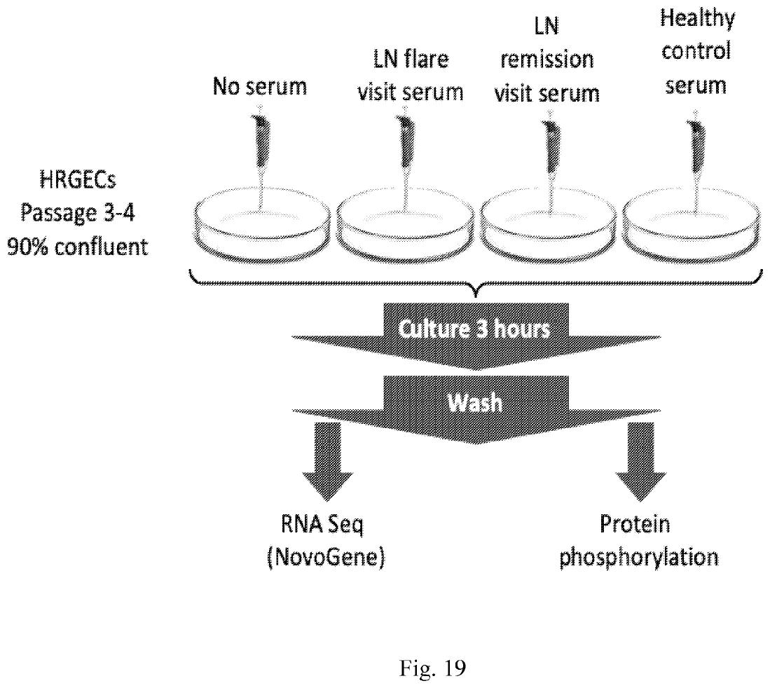

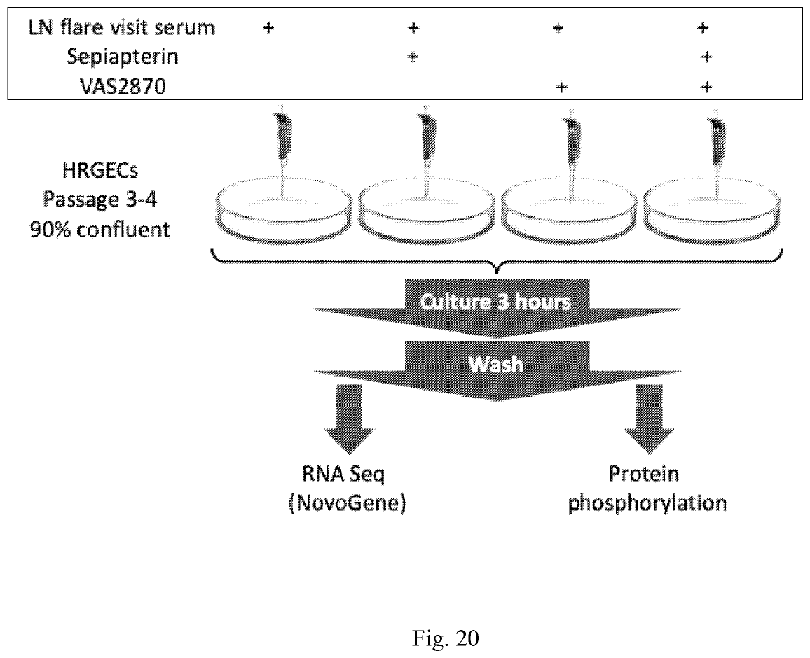

The studies described demonstrate that serum from LN patients with active disease induce an inflammatory endothelial cell phenotype with increased neutrophil migration and adhesion. It is also published that sepiapterin effectively improves endothelial function by increasing EC NO (Jones Buie, J. N. et al., 2019, Medicine, 6:e000294). The primary source of NO in EC is endothelial NO synthase (eNOS), which is demonstrated as important for modulating crescentic and necrotic glomerular disease in the previously published work (Gilkeson, G. S. et al., 2013, PLoS One, 8(5)). Therefore, these studies are designed to differentiate pathways activated by lupus serum during disease activity over disease inactivity. Pooled serum from at least 10 patients followed longitudinally during visits with LN flare and during remission as well as pooled serum from at least 10 healthy, non-connective tissue disease hypertension-free controls is used for experiments. HRGEC is treated with active LN, remission LN, or control serum as in FIG. 9 and FIG. 10. Cell lysates are collected for RNA seq analysis and candidate protein phosphorylation (FIG. 19). The following experiments secondarily determine which of these pathways are redox-regulated (targeted by compounds that improve eNOS function (sepiapterin) and reduce NADPH oxidase activity (VAS2870) or a combination of both compounds. Pooled serum from at least five patients in LN flare is cultured with added sepiapterin or VAS2870 per published protocols and preliminary studies (Jones Buie, J. N. et al., 2019, Medicine, 6:e000294), and lysates are collected for RNA seq and protein phosphorylation as above (FIG. 20).

Some of the pathways activated in FIG. 8 are modulated by existing medications (hydroxychloroquine and angiotensin-converting enzyme or angiotensin receptor blocker therapy, for instance). Knowledge of pathways activated and which of these pathways are redox-regulated is important in determining specific therapies for these targeted pathways. Using a high throughput 96 well format, the importance of activated pathways is confirmed with pharmacologic inhibition and siRNA experiments with cells cultured with pooled active LN serum as in FIG. 19. NO and SO production with each treatment is determined as below. The studies provide the specific mechanistic rationale for targeting redox pathways to improve endothelial dysfunction. They also offer important information on which drugs may be re-purposed to specifically to improve endothelial function.

LN Case and Control Populations

In the preliminary and published studies, patients with proliferative (ISN/RPS class III or IV) LN were identified during active disease at the time of blood collection and have been observed in prospective longitudinal cohorts until disease is inactive. Active disease was defined by proteinuria above 0.5 g/g of creatinine or LN by renal biopsy within one month of blood collection. Disease was deemed active if the treating provider prescribed induction therapy for active LN. Inactive disease was defined as a urine protein/creatinine level of 0.5 g/g or less in the absence of increases in glucocorticoid or immunosuppressive therapy or end-stage renal disease or chronic kidney disease stage III or above. Unrelated healthy controls were screened using the connective tissue disease questionnaire (CSQ (Karlson, E. W. et al., 2005, Lupus, 14:832-6)). Those that screened negative by survey and a negative chart review for connective tissue disease were included in this study. They were not included if they had a history of cardiovascular disease or active infection. Serum from these individuals was collected and stored in aliquots at −80° C. until the time of culture as in the preliminary studies.

Human Renal Glomerular Endothelial Cell (HRGEC) Culture

Primary human glomerular endothelial cells are used to mimic the in vivo LN environment as much as possible. Limited numbers of passages prevents excessive phenotype drift. Human Renal Glomerular Endothelial Cells (HRGECs) (ScienCell, Carlsbad, Calif.) are cultured according to manufacturer's protocol in fibronectin-coated flasks in complete growth media supplemented with 5% fetal bovine serum (FBS), 1% endothelial cell growth supplement, and 1% penicillin/streptomycin solution. Cells are incubated at 37° C. in a humidified atmosphere in the presence of 5% CO₂ and sub-cultured upon reaching 90% confluency. HRGECs are used at passage 3-4.

Serum Co-Culture

HRGECs are plated at 4×10⁴ cells per well of a 48-well fibronectin-coated plate and cultured in complete growth media at 37° C. in a humidified atmosphere in the presence of 5% CO₂. Upon confluence, complete growth media is removed, and cells washed three times in ECM. After completely aspirating media, cells are treated with 10% serum (SLE or control, no heat inactivation) in ECM for 3 hours at 37° C. in a humidified atmosphere in the presence of 5% CO₂. Before endpoints, cells are washed to remove serum.

Phosphorylation

In the published and preliminary studies, many of the changes in redox status in endothelial cells occurs within a few minutes of treatment. Therefore, it is suspected that redox sensitive changes in phosphorylation in pathways that activate NADPH oxidase activity. Cells treated as in FIG. 19 and FIG. 20 are analyzed for protein phosphorylation using the Proteome Profiler Human Phospho-Kinase Array Kit from R&D Systems. This membrane-based sandwich immunoassay array allows detection of relative levels of human protein kinase phosphorylase and in parallel, thus reducing inter-assay variability. The assay detects phosphorylation of 44 proteins, including Src, Akt, p38 MAPK, ERK1/2, cJun, eNOS (ser1177), PDGFRβ, and PLCγ1 that are relevant to redox-sensitive pathways.

RNA Seq Analysis

HRGECs are plated at 2×10⁵ cells per 100 mm dish and cultured in complete growth media at 37° C. in a humidified atmosphere in the presence of 5% CO₂. Upon confluence, complete growth media is removed, and cells washed three times in ECM. After completely aspirating media, cells undergo treatments per the general protocol (FIG. 19 and FIG. 20), Cells are washed and treated with trypsin. Cells are scraped, pelleted, washed in PBS, and 200 μl RNA later is added and samples are snap frozen and stored at −80° C. RNA are isolated later using and RNAeasy Mini Kit per the manufacturer's instructions. RNA concentration and quality are read using a NanoDrop spectrophotometer at 260/280 nm and gel of electrophoresis to ensure no degradation or DNA/protein contamination per NovoGene requirements. Isolated RNA (1-2 μg in 20 μL) are sent to Novogene for analysis. The services are performed on contract for transcriptome sequencing and quantification using the Illumina NovoSEQ and HiSEQ platforms with a paired-end 150 bp sequencing strategy. A bioinformatics pipeline is used to ensure quality, targeting greater than 80% with a sequencing quality score above Q30. After data quality checks are complete, sequences are mapped to the reference genome and gene expression quantified. Differential expression analysis includes a protein-protein interaction analysis and a transcription factor analysis as described (Zhao, X. et al., 2017, Nat. Med., 23:337-46). As an alternative or complimentary assay to confirm protein expression of differentially expressed proteins, a bead array or ELISA analysis of cell lysate material is performed as described (Lou, N. et al., 2017, Mol. Immunol., 81:59-66; Lennard Richard, M. L. et al., 2014, J. Immunol., 193:2661-8; Lennard Richard, M. L. et al., 2015, Mol. Immunol., 63:566-73; Sato, S. et al., 2014, Arthritis & Rheumatology, 66:3436-44; Lelievre, E. et al., 2002, J. Biol. Chem., 277:25143-51; Wilson, L. A. et al., 2005, FASEB, 19:2085-7). Biomarkers identified that increase/decrease with LN flare serum and sepiapterin treatment are targeted. Candidate RTPCR is performed as described (Jones Buie, J. N. et al., 2019, Medicine, 6:e000294; Lennard Richard, M. L. et al., 2014, J. Immunol., 193:2661-8).

Statistical Analysis Plan and Sample Size Justification

The primary goal is to conduct DE (differential expression) analysis of gene expression profiles measured as fold change (FC) between healthy controls, LN patients during flare, and LN patients during remission. Differential expression that appears to be redox regulated are also tested. The combined comparisons are informative to discover factors associated with an inflammatory LN EC phenotype that are redox regulated. Samples for LN patients with active flare and during remission are pooled (10 patient samples/pooled sample). For RNAseq analysis, samples from 10 healthy controls, 10 pooled LN in remission, and 10 pooled LN in active flare are included. The sequencing depth include approximately 10 million reads per sample as a depth of 10 million reads has been shown to provide coverage of at least 10 reads for 90% of all genes (Hart, S. N. et al., 2013, J. Comput. Biol., 20:970-8). The detectable FC for these sample sizes was estimated using the RNASeqPower library available in Bioconductor (Hart, S. N. et al., 2013, J. Comput. Biol., 20:970-8). Sample size in RNA seq experiments depends on the read count for a transcript, the coefficient of variation (CV) of counts within experimental condition, FC (effect size), and type I and type II error rates. The CV is set at 0.4 which has been shown to be an appropriate estimate for human studies) (McCarthy, D. J, et al., 2012, Nucleic Acids Res., 40:4288-97; Robinson, M. D> et al., 2010, Bioinformatics, 26:139-40) and is assumed to be equal across groups. Power is fixed at 80% and type I error rate is conservatively fixed at α=0.05/20,000=2.5×10⁻⁶. Among transcripts with 10 reads, FC values of 3.54 or greater are detectable with 80% power. For transcripts with 200 read counts FC values 2.74 are detectable with 80% power. The power analysis is conservative since identification of genes of interest are based on a Benjamini-Hochberg FDR approach. For validation, quantitative PCR (qPCR) is performed to confirm changes in the expression pattern in all LN remission vs. LN flare, LN remission vs. control, and LN flare vs. control samples.

Targeting Redox-Regulated Pathways to Improve Endothelial Cell Function

Pathways revealed by pathway analysis of data from Example 3 are validated as functional pathways leading to changes in NO and SO production. First, pharmacologic inhibition experiments are performed in a high-throughput assay as in the preliminary studies and as described in per Example 3. It is expected that the p38 MAPK pathway activates to induce NADPH oxidase SO production. Upstream of this activation, several pathways are interrogated with added inhibitors (Table 3). Significant results are confirmed with siRNA studies (Lou, N. et al., 2017, Mol. Immunol., 81:59-66; Lennard Richard, M. L. et al., 2015, Mol. Immunol., 63:566-73; Sato, S. et al., 2014, Arthritis & Rheumatology, 66:3436-44). The effect of NADPH oxidase activity on NO and SO production is determined with use of a specific inhibitor VAS2870. Similarly, the effect of NOS uncoupling on NO and SO production is determined by adding sepiapterin.

TABLE 3
Potential inhibitors to target identified pathways

| Target | inhibitor | IC50 | |
|---|---|---|---|
| NADPH Oxidase | VAS2870 | 1-2 | μM |
| i/e/nNOS | L-NAME | 4.4 μM (iNOS) | |
| iNOS | L-NIL | 3.3 | μM |
| uncoupled eNOS | sepiapterin | 5 μM sufficient | |
| p38α | AL 8697 | 6 | nM |
| p38α and p38β2 | EO1428 | 74 | nM |
| myD88 | MyD88 peptide | ~100 | μM |
| TLR 7/9 | hydroxychloroquine | ~4 | μM |
| Syk (in Fc receptor complex) | Tamatinib/R406 | 41 | nM |

Quantification of Intracellular NO with DAF-FM Diacetate

HRGECs are plated at 3×10⁴ cells per well in a 96-well black-walled plate and cultured in complete growth media at 37° C. in a humidified atmosphere with 5% CO₂. Upon confluence, complete growth medium is removed, and cells washed in ECM-prf (phenol red free). Cells are incubated with 2 μM DAF-FM Diacetate for 60 minutes at 37° C., then cells washed in ECM-prf. After aspirating medium, cells undergo treatment with 10% serum (lupus or control) with or without 5 μM L-Sepiapterin (SantaCruz), 25 μM VAS2870 (Sigma Aldrich) or both. 0.6 mM DPTA NONOate (Abcam) is a positive control, while L-NAME (100 μM) is a negative control. Because DAF-FM also fluoresces with peroxynitrite, a urate control (5 μM) is added to each experimental condition to scavenge peroxynitrite. Plates are incubated for 30 minutes at 37° C. and read at 485/528 excitation/emission on a microplate reader (BioTek HT Synergy). Pharmacologic and siRNA inhibition experiments are also performed to confirm pathways identified in Example 3.

Quantification of ROS Levels with CM-H₂DCFDA

HRGECs are plated, cultured to confluence, and washed as in the DAF experiments above. NO and ROS expression are correlated with to RNAseq results to suggest redox regulation of gene expression. After completely aspirating media, cells undergo various treatments: 10% serum (LN (active and inactive time points in the same subjects) or non-hypertensive control), 1 mM uric acid (Alfa Aesar), or 4.5 mU/mL xanthine/xanthine oxidase (Millipore Sigma) positive control, or 10 μM allopurinol (CaymanChem) negative control in ECM-prf for 30 minutes at 37° C. in a humidified atmosphere in the presence of 5% CO2. Cells are washed three times in ECM-prf, then 5 μM CM-H2DCFDA (Invitrogen) is added followed by incubation for 30 minutes at 37° C. Cells are washed again three times in ECM-prf, then re-suspended in ECM-prf. Excitation/emission is read at 485/528 nm on a microplate reader (BioTek HT Synergy). Treatment with serum and inhibitors/sepiapterin are as described above.

siRNA Transfections

These experiments are used to confirm pathways identified in pathway analysis and inhibitor studies. SiRNA transections are carried out per the previously described methods (Lou, N. et al., 2017, Mol. Immunol., 81:59-66). Briefly, lipofectamine is used to transect endothelial cells following the manufacturer's instructions. Cells are cultured for 24 hours before culture with serum.

These Examples support targeting endothelial dysfunction with sepiapterin to reducing end organ inflammation in immune complex-mediated renal disease. Inflammatory infiltrates are reduced by sepiapterin therapy without changes in IgG and C3 stain. In keeping with the eNOS genetic ablation studies (Gilkeson, G. S. et al., 2013, PLoS One, 8:e64650), restoration of EC function improved histopathologic features of immune complex nephritis such as crescents and necrosis.

### Example 6: Effects of CSNA-001 (Sepiapterin) on Systolic Blood Pressure (SBP)

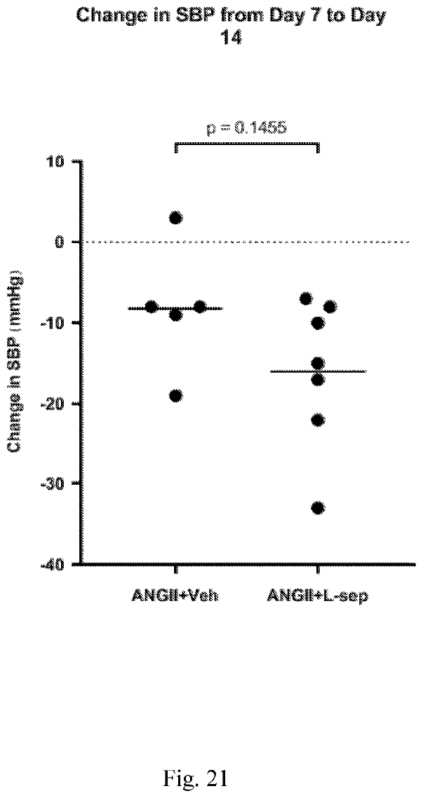

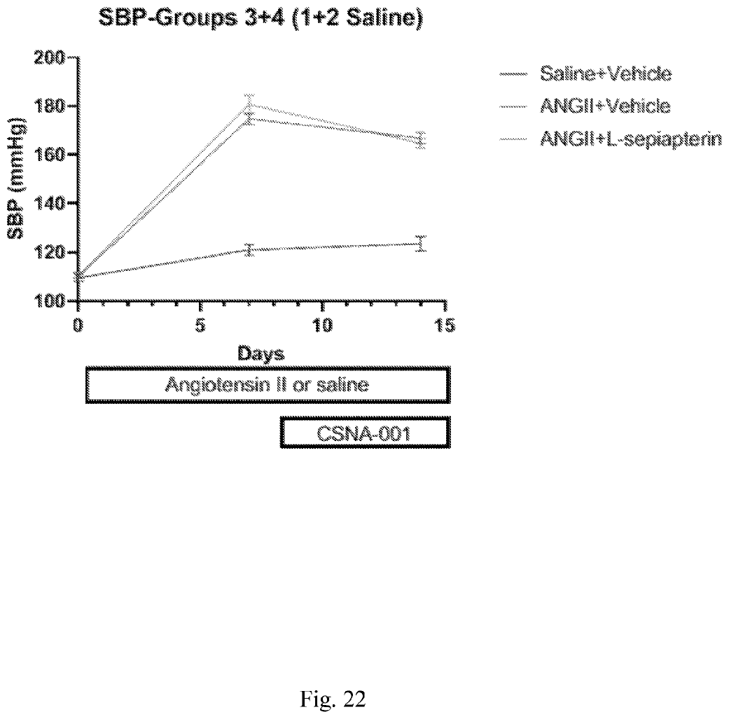

Hypertension was created in some BALB/c mice with Angiotensin II minipumps. Mice were treated half with CSNA-001 (L-sep) 8 days after the pumps were placed and baseline SBP checked. The decrease in systolic BP was significant in the treated mice but not the untreated (FIG. 21 and FIG. 22). In addition, there were significant differences in mean blood pressure responses to acetyl choline between treated and untreated mice. This suggests that 20 mg/kg daily is sufficient to have a vascular endothelial effect on blood pressure. This study demonstrates the ability to TREAT hypertension rather than simply PREVENT it in such a model, and demonstrates the use of cuff pressure as an endpoint (rather than isolated vessels (as in Keller A C, Knaub L A, Scalzo R L, et al. Sepiapterin Improves Vascular Reactivity and Insulin-Stimulated Glucose in Wistar Rats. Oxid Med Cell Longev. 2018; 2018:7363485). The differences in reduction were about 8 mmHg, which is clinically significant.

### Example 7: Dosage Effects for Treatment of Lupus Nephritis

Clinical trials have been developed to determine the effective dosage for treatment of lupus nephritis. Of 512 subjects to meet ACR criteria in a longitudinal cohort that have weight data on the first visit, the mean weight is 77 kg with a standard deviation of 22. Similar results were obtained if only the 466 females were included.

Of 643 patients who meet ACR criteria in the cohort, 308 have a history of renal disease. 268 are still living. Most of the patients in this cohort have agreed to contact.

Using a strict e-phenotype in the research data warehouse of all patients, 720 meet criteria for lupus nephritis and are still alive. 530 were also coded as having glomerular disease. Of note, the same number are on some immunosuppressant (mycophenolate, azathioprine, cyclosporine, or tacrolimus) typically used for lupus nephritis. The historic arrival rate (last 3 years) of new patients that fit these criteria is approximately nine per month.

### Study Design:

### Endpoints

Effect size—
Primary endpoints:
   flow mediated dilation (Endopat).
      Do 2 baseline measures to reduce the effect of familiarity with the device and setting rather than using crossover design
   Circulating cell BH2/BH4 pre and post
Secondary endpoints
   Blood pressure reduction
   Biomarkers urine and serum of ROS and cytokine/chemokines
   Flow T cell subsets (data on NO in T cell function)
SLEDAI—flares (SFI)

Dose Finding
Dose escalation with adaptive design if effect not seen in first group of patients IF pharmacodynamic endpoint of BH2/BH4 not met.

Safety
Blood pressure, hypotension

Controlled before-and-after Phase II proof of concept trial

Study population (n=10)

ACR or SLICC Criteria for SLE

Lupus nephritis history
Urine prot/creat>0.5 g/g in two readings at least 1 month apart OR ISN/RPS histopathologic criteria for lupus

Stable disease with no BILAG A or B in any system for the past 4 weeks

Stable prednisone (or equivalent corticosteroid) dose≤20 mg/day for ≥4 weeks

prior to study entry

Age 18-85 years

Ability to give informed consent

### Exclusion

Malignancy (except nonmelanoma skin cancer)

Active infection

Tobacco smoking (active)

Pregnancy

Receiving nitroglycerin or other vasodilating nitrates

### Intervention

20 mg/kg/day CNSA-001 or placebo in crossover design (consider not doing this due to lag in effect).

Duration 2 weeks

Compound estimate:

At 20 mg/kg daily for two weeks at an estimated 77 kg average weight for 10 patients=15.4 grams. Add additional 50% waste=23.1 grams.

500 mg capsules: If this were assumed, the maximum variation would be in those of lower weight (60 kg round down would result in −20% of drug). Another option is to round up. Then, a 60 kg or 80 kg person would receive a 25% increase (see Table 4 below).

TABLE 4

| | | round | differ- | % | round | differ- | % |
|---|---|---|---|---|---|---|---|
| weight | dose | down | ence | difference | up | ence | difference |
| 60 | 1200 | 1000 | −200 | −20.00% | 1500 | 300 | 25.00% |
| 65 | 1300 | 1500 | 200 | 13.33% | 1500 | 200 | 15.38% |
| 70 | 1400 | 1500 | 100 | 6.67% | 1500 | 100 | 7.14% |
| 75 | 1500 | 1500 | 0 | 0.00% | 1500 | 0 | 0.00% |
| 80 | 1600 | 1500 | −100 | −6.67% | 2000 | 400 | 25.00% |
| 85 | 1700 | 1500 | −200 | −13.33% | 2000 | 300 | 17.65% |
| 90 | 1800 | 1600 | −200 | −12.50% | 2000 | 200 | 11.11% |

TABLE 5
Draft study visit document

| | | | | study drug | | blood | urine | cells | cells |
|---|---|---|---|---|---|---|---|---|---|
| day | visit | screen | SLEDAI | dispensed | EndoPat | (biomarkers) | (biomarkers) | (BH2/4) | (Tcell) |
| “−(1-28)” | screen | x | x | | | | x | | |
| 0 | 1 | | x | 7 days | x | x | x | x | x |
| 7 | 2 | | | 7 days | | | | | |
| 14 | 3 | | x | pill count | x | x | x | x | x |
| 28 | 4 | | x | | x | x | x | x | x |

| day | CMP | C3 | C4 | dsDNA | CBC | UA | UrPr/Cr | vitals | room |
|---|---|---|---|---|---|---|---|---|---|
| “−(1-28)” | x | x | x | x | x | x | x | x | x |
| 0 | x | x | x | x | x | x | x | x | x |
| 7 | | | | | | | | x | x |
| 14 | x | x | x | x | x | x | x | x | x |
| 28 | x | x | x | x | x | x | x | x | x |

### Example 8: Effect of Sepiapterin on Hypertension

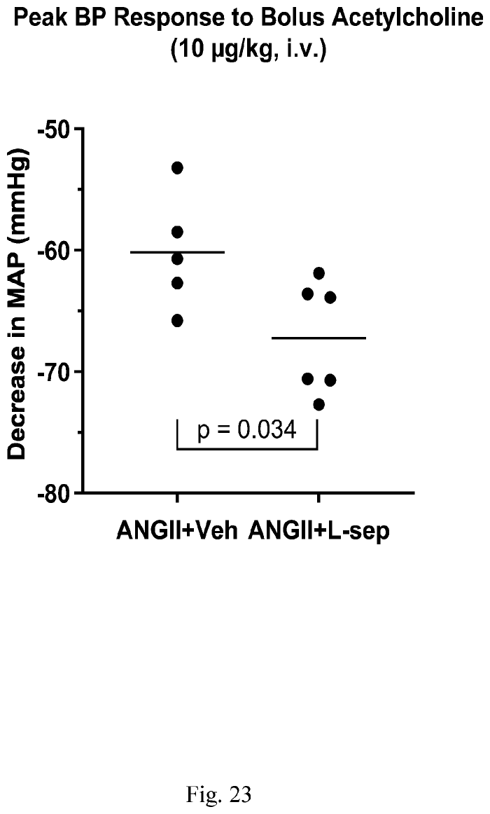

To determine the effect of sepiapterin in a mouse model of hypertension, C57B6/J mice were implanted with an angiotensin II minipump for 2 weeks to induce hypertension. The animals were then treated with either sepiapterin 10 mg/kg/d by gavage or vehicle. A tail cuff measure of blood pressure was taken followed by injection of acetylcholine and a second tail cuff measure of blood pressure. FIG. 23 shows the difference in pressure, and demonstrates that sepiapterin improves endothelial function in the angiotensin II model of hypertension.

### Example 9: Lupus Serum Induces Inflammatory Interaction with Neutrophils in Human Glomerular Endothelial Cells

SLE is associated with endothelial cell dysfunction (ECD). Understanding how ECD leads to neutrophil infiltration into glomeruli is essential to finding therapeutic targets for SLE. The experiments presented herein were conducted to determine the effect of SLE serum from patients with active disease to induce neutrophil adhesion to and chemotaxis towards glomerular endothelial cells and factors induced by serum that associate with neutrophil chemotaxis. Patients with SLE had serum collected during paired longitudinal visits with lower and higher activity. 13 patients with SLE (5 SLE, 5 SLE with hypertension (HTN) and 3 SLE lupus nephritis (LN) and HTN), and 10 healthy controls (5 with and 5 without HTN) were examined. The adhesion of neutrophils to serum-treated human renal glomerular endothelial cells (HRGECs) or chemotaxis of neutrophils towards conditioned media from serum-treated HRGECs was determined, and levels of cytokines in this conditioned medium were quantified. Pathway analysis of cytokines induced by SLE and LN serum that associated with neutrophil migration was performed.

It is demonstrated herein that HRGECs treated with SLE serum induced significantly greater neutrophil chemotaxis and adhesion compared with control serum. When examining specific cohorts, SLE HTN and LN HTN promoted greater neutrophil chemotaxis than control serum, while SLE HTN and LN HTN promoted greater chemotaxis than SLE serum. Serum from active disease visits promoted neutrophil chemotaxis and adhesion over paired inactive visits. Levels of platelet-derived growth factor-BB, interleukin (IL)-15 and IL-8 secreted by SLE serum-treated HRGECs positively correlated with neutrophil chemotaxis. Pathway analysis suggested that LN serum induced pathways important in endoplasmic reticulum and oxidative stress. Thus, the experiments show that SLE serum induces expression of mediators by HRGECs that promote neutrophil chemotaxis and adhesion, which increases during disease activity, and associates with factors common to pathways of endoplasmic reticulum and oxidative stress. These findings highlight the potential importance of serum factor-induced ECD in SLE and LN.

SLE is a chronic autoimmune disease characterized by humoral autoimmunity, immune complex formation and innate and cellular immune responses. Lupus nephritis (LN) and renal failure in LN are important predictors of mortality in patients with SLE (Almaani et al., Clin J Am Soc Nephrol 2017; 12:825-35; Danila et al., Rheumatology 2009; 48:542-5; Rijnink et al., Clin J Am Soc Nephrol 2017; 12:734-43; Appel et al., Am J Med 1987; 83:877-85; Maroz et al., Am J Med Sci 2013; 346:319-23). The most aggressive form of LN is characterized by infiltration of neutrophils, fibrinoid necrosis, fibrocellular crescent formation and rupture of basement membranes (Weening et al., J Am Soc Nephrol 2004; 15:241-50; Markowitz et al., Curr Opin Nephrol Hypertens 2009; 18:220-5; Kiremitci et al. ScientificWorldJournal 2014; 2014:1-10). Therefore, the processes that lead to neutrophil infiltration into glomeruli are logical targets for therapy.

During homeostasis, the endothelium acts as a modulator of inflammatory responses such as cellular chemotaxis, rolling, adhesion and transmigration into tissue. When activated by inflammatory factors during pathology, the endothelium expresses chemokines and cellular adhesion molecules (Bendall et al., Antioxid Redox Signal 2014; 20:3040-77; Atehortúa et al., Mediators Inflamm 2017; 2017:1-12). Endothelial cell dysfunction (ECD), the clinical and cellular manifestation of an activated endothelium, associates with SLE disease activity (Pramanik et al., Indian J Rheumatol 2011; 6:178-84). It has been reported that endothelial nitric oxide synthase (eNOS) is essential for modulating the formation of crescentic and necrotic glomerular lesions in a murine model of LN (Gilkeson et al., PLoS One 2013; 8:e64650). Here, it is examined whether factors in lupus serum would lead to glomerular ECD in a LN disease activity-dependent manner. Therefore, the first goal of the current study was to determine the effects of lupus serum on human glomerular endothelial function as assessed by neutrophil adhesion to and migration towards serum-treated human renal glomerular endothelial cells (HRGECs). The second goal was to determine potential factors and pathways leading to these cellular ECD phenotypes.

The methods used in these experiments are now described.

### Subjects

In a prospective longitudinal cohort, patients with SLE were evaluated during standard of care visits. SLE Disease Activity Index (SLEDAI) scores (Romero-Diaz et al., Arthritis Care Res 2011; 63:S37-46) were determined at each visit. Serum collected at each standard of care visit was stored at −80° C. Inclusion criteria from this larger cohort into this study were 1) presence of four or more American College of Rheumatology (ACR) diagnostic criteria for SLE (Hochberg, Arthritis Rheum 1997; 40:1725), 2) serum stored from two or more longitudinal, paired visits in which the SLEDAI indicating higher and lower disease activity differed by a score of ≥4 and 3) no cardiovascular disease by SLE International Collaborating Clinics Damage Index elements (Gladman et al., Arthritis Rheum 1996; 39:363-9). Patients with SLE were then sorted into the following groups: 1) SLE with no nephritis (no ACR renal criteria and no visits with a urine protein/creatinine ratio>0.5 g/g) and no hypertension (HTN), 2) SLE with no nephritis but with HTN by history and 3) SLE with nephritis (active at one visit by SLEDAI renal elements or biopsy proven) and HTN. SLE demographic data are summarized in Table 5. HTN was defined by the clinician as values>140/90 mm Hg for patients without renal disease and as >130/80 for patients with LN, prior to treatment with antihypertensive medications. SLE patient clinical data are summarized in Table 6 and medications are summarized in Table 7. Healthy controls, matched for age, sex and race, were selected if they had no cardiovascular disease by history and had a negative Connective Tissue Disease Screening Questionnaire (Karlson et al., Ann Epidemiol 1995; 5:297-302). Controls were divided into those with and without HTN. Control subject clinical data are summarized in Table 8. Additional healthy controls without connective tissue disease or cardiovascular risk factors/events were selected from a convenience population as donors for prospective neutrophil isolation.

TABLE 5
Subject demographics

| | | | | | SLE |
|---|---|---|---|---|---|
| | | Control | | SLE | LN |
| | Control | HTN | SLE | HTN | HTN |
| Cohort size (n) | 5 | 5 | 5 | 5 | 3 |
| Age (mean | 40 ± 12 | 54 ± 7 | 36 ± 6 | 47 ± 14 | 40 ± 10 |
| years ± SD) | | | | | |
| Female sex (%) | 100 | 100 | 100 | 100 | 100 |
| African-American (%) | 60 | 100 | 80 | 40 | 66 |

TABLE 6
Clinical Characteristics - lupus subjects

| | SLE | | SLE HTN | | SLE LN HTN | |
|---|---|---|---|---|---|---|
| | (mean ± SD) | | (mean ± SD) | | (mean ± SD) | |
| | Inactive | Active | Inactive | Active | Inactive | Active |
| SLEDAI | 2.0 ± 2.0 | 9.0 ± 1.4 | 2.0 ± 2.8 | 10.4 ± 1.5 | 0.6 ± 1.1 | 8.0 ± 2.0 |
| Anti-dsDNA antibody | 15 ± 18 | 66 ± 131 | 44 ± 88 | 43 ± 88 | 14 ± 15 | 17 ± 25 |
| Urine protein/ | 0.08 ± 0.02 | 0.16 ± 0.15 | 0.16 ± 0.16 | 0.18 ± 0.09 | 0.27 ± 0.17 | 3.36 ± 4.35 |
| creatinine ratio | | | | | | |
| C3 complement | 102 ± 20 | 100 ± 42 | 114 ± 14 | 106 ± 7 | 114 ± 2 | 105 ± 23 |
| (mg/dL) | | | | | | |
| C4 complement | 15 ± 10 | 12 ± 7 | 20 ± 11 | 18 ± 14 | 22 ± 9 | 25 ± 9 |
| (mg/dL ± SD) | | | | | | |
| Antiphospholipid | 0% | 0% | 20% | 20% | 0% | 0% |
| syndrome (% of total | | | | | | |
| patients) | | | | | | |
| Systolic blood | 117 ± 11 | 120 ± 9 | 140 ± 21 | 154 ± 20 | 112 ± 12 | 122 ± 18 |
| pressure (mm Hg) | | | | | | |
| Diastolic blood | 73 ± 8 | 75 ± 11 | 88 ± 21 | 97 ± 21 | 75 ± 5 | 74 ± 13 |
| pressure (mm Hg) | | | | | | |
| BMI | 30 ± 15 | 31 ± 15 | 29 ± 7 | 29 ± 7 | 31 ± 6 | 30 ± 5 |
| Blood glucose | 95 ± 18 | 87 ± 6 | 105 ± 15 | 100 ± 25 | 79 ± 10 | 85 ± 13 |
| (mg/dL) | | | | | | |
| Total cholesterol | 136 ± 6 | 203 ± 55 | 138 ± 28 | 153 ± 42 | 227 ± 6 | 239 ± 114 |
| (mg/dL) | | | | | | |
| LDL (mg/dL) | 77 ± 5 | 125 ± 23 | 72 ± 28 | 80 ± 25 | 149 ± 14 | 165 ± 90 |
| HDL (mg/dL) | 45 ± 4 | 62 ± 28 | 51 ± 16 | 59 ± 21 | 46 ± 17 | 109 ± 65 |
| Triglycerides (mg/dL) | 71 ± 10 | 76 ± 23 | 74 ± 32 | 77 ± 25 | 109 ± 65 | 141 ± 38 |

TABLE 7
Medications - lupus subjects

| | SLE | | SLE HTN | | SLE LN HTN | |
|---|---|---|---|---|---|---|
| | Inactive (%) | Active (%) | Inactive (%) | Active (%) | Inactive (%) | Active (%) |
| On immunosuppressives | 40 | 60 | 80 | 80 | 100 | 100 |
| On NSAIDs | 0 | 20 | 40 | 80 | 67 | 67 |
| On ACE inhibitors | 0 | 20 | 80 | 60 | 67 | 100 |
| On prednisone | 40 | 80 | 40 | 20 | 33 | 33 |
| Prednisone >10 mg | 0 | 20 | 0 | 0 | 0 | 33 |
| On Plaquenil | 80 | 100 | 100 | 100 | 67 | 67 |

TABLE 8
Clinical Characteristics - control subjects

| | Control | Control HTN |
|---|---|---|
| | (mean ± SD) | (mean ± SD) |
| Systolic blood pressure (mm Hg) | 114 ± 24 | 143 ± 13 |
| Diastolic blood pressure (mm Hg) | 63 ± 9 | 81 ± 2 |
| BMI | 30 ± 12 | 46 ± 14 |
| Blood glucose (mg/dL) | 102 ± 36 | 105 ± 17 |
| Total cholesterol (mg/dL) | 181 ± 19 | 209 ± 40 |
| LDL (mg/dL) | 118 ± 19 | 136 ± 35 |
| HDL (mg/dL) | 64 ± 5 | 54 ± 12 |

### HRGEC Cell Culture

Primary human renal glomerular endothelial cells (HRGECs) (Cat #4000, ScienCell, Carlsbad, Calif., USA) were cultured according to manufacturer's protocol. Briefly, HRGECs were cultured in fibronectin-coated flasks in complete growth media (Cat #1001, Endothelial Cell Medium (ECM)) supplemented with 5% fetal bovine serum (FBS), 1% endothelial cell growth supplement and 1% penicillin/streptomycin solution (ScienCell). Cells were incubated at 37° C. in a humidified atmosphere in the presence of 5% CO₂ and subcultured on reaching 90% confluency. Once confluent, cells were rinsed in Dulbecco's phosphate buffered saline (DPBS). Then 10 mL of DPBS with 1 mL 0.25% trypsin/EDTA (ScienCell) solution was added. Cells were incubated at 37° C. for 2 min or until cells were rounded. Trypsin/EDTA and cell solution was added to a conical with 5 mL FBS. Flask was incubated for another 2 min; 10 mL tyrpsin neutralization solution (TNS) solution (ScienCell) was then added to flask to collect remaining cells, and then transferred to conical with cells and FBS. Centrifuge cells at 1000 rpm for 5 min. Cells were counted and checked for viability (>90%) using Trypan Blue. Cells were then resuspended in complete growth media. HRGECs were used at passage 3-4. Based on our cell culture experience, this passage number was selected for its retention of the endothelial cell phenotype (based on CD31 staining, data not shown) and for enhanced cell viability.

### Neutrophil Isolation

Blood from healthy, non-hypertensive controls without cardiovascular disease was collected into K₂EDTA tubes (BD) at room temperature. Less than 1 hour after blood was collected, immunomagnetic negative bead selection was performed using EasySep Neutrophil Isolation kit (StemCell Technologies, Cambridge, Mass., USA) and Easy 50 magnet (StemCell Technologies) according to manufacturer's protocol. The typical neutrophil content (CD66b+CD16+) of the final isolated fraction is 94.0%±3.7% according to manufacturer, and our lab confirmed purity by Wright-Giemsa stain. Phosphate-buffered saline (PBS) without Ca²⁺/Mg²⁺ (Thermo Fisher Scientific, Waltham, Mass., USA) supplemented with 1 mM EDTA (VWR Life Science, Radnor, Pa., USA) was added to the RapidSpheres. After isolation of neutrophils, viability>90% was confirmed via Trypan Blue Solution and cells were immediately used for chemotaxis and adhesion assays. Assays were completed within 3 hours of isolation to ensure continued viability of neutrophils.

### Neutrophil Labelling

Neutrophils were labelled with 3 μM Calcein AM solution (Thermo Fisher Scientific) per the manufacturer's instructions (Gibco, Thermo Fisher Scientific). Calcein AM is a cell-permeant dye used to determine cell viability, and in live cells the non-fluorescent Calcein AM is converted to a green fluorescent form by intracellular esterases. Viability of cells (>90%) is further confirmed using Trypan blue. For adhesion assays, the dyed cells were passed through a 40 μm sterile filter to remove clumps, and washed in RPMI-1640 without phenol red.

### Neutrophil Migration Assay

HRGECs were plated at 4×10⁴ cells per well of a 48-well fibronectin-coated plate in complete growth media at 37° C. with 5% CO₂. On confluence, cells were washed three times in ECM. Cells were then cultured with 10% serum (SLE or control) in ECM for 3 hours and then washed three times in ECM. Cells were serum starved overnight in ECM. The resulting conditioned media (CM) was then collected and passed through a 0.2 μm pore membrane to remove any cellular debris and frozen at −80° C. for later use.

Transwell membrane chambers with 3 μm pores from a 96-well Migration Plate (Corning, Corning, N.Y., USA) were equilibrated in RPMI for 1 hour at 37° C. Then RPMI was removed. CM or negative (CM from serum-free treatment)/positive (10⁻⁷ M interleukin (IL)-8 (R&D Systems, Minneapolis, Minn., USA)) controls were added to the 96-well black-walled receiver wells (Corning); 1.5×10⁵ neutrophils in ECM were then added to the upper chamber of each insert and incubated for 1 hour to allow for neutrophil migration toward the conditioned medium. Cells that migrated through the membrane into the CM were then labelled with Calcein AM and incubated for 1 hour at 37° C. as described above. Fluorescence intensity was then measured from the bottom of the plate at 485 nm excitation, 520 nm emission using a fluorescence microplate reader (Biotek HT Synergy). A standard curve of Calcein AM-labelled neutrophils (using 1.5×10⁵, 1.0×10⁵, 5.0×10⁴, 2.5×10⁴, 1.25×10⁴, 0.625×10⁴ and 0 neutrophils) was created and fluorescence intensity was quantified on the microplate reader. The linear equation was then calculated and fluorescence intensity of samples could then be input into the linear equation to determine neutrophil cell number. The fluorescence intensity of migrated neutrophils in the lower chamber was measured as described (Jones Buie et al., Lupus Sci Med 2019; 6:e000294). All values were normalized to untreated controls.

### Cytokine Quantification

CM was collected as indicated above for neutrophil chemotaxis migration assay. HRGECs were plated at 4×10⁴ cells per well of a 48-well fibronectin-coated plate in complete growth media at 37° C. with 5% CO₂. On confluence, cells were washed three times in ECM. Cells were then cultured with 10% SLE serum (from patients with paired inactive and active disease visits, n=7) in ECM for 3 hours and then washed three times in ECM. Cells were serum starved overnight in ECM. The resulting CM was then collected and passed through a 0.2 μm pore membrane to remove any cellular debris. CM was stored at −80° C. after being split into separate vials to be used for either migration assays or cytokine quantification, so that cytokine levels in CM could be directly correlated with the ability of the same CM to promote neutrophil chemotaxis. After collection, CM was stored at −80° C. then shipped on dry ice to Eve Technologies for analysis. Human Cytokine Array 42-Plex with IL-18 (Eve Technologies, Alberta, Canada) and Human Supplemental Biomarker Array 10-Plex (Eve Technologies) were used to quantify cytokine concentrations. Correlation analysis with chemotaxis was then assessed. Cytokines of interest were confirmed via ELISA (R&D Systems; RayBiotech, Peachtree Corners, Ga., USA).

### Neutrophil Adhesion Assay

HRGECs were cultured in 48 well fibronectin-coated plates at 80 000 cells per well in complete growth media until confluent. HRGECs were washed, serum starved for 1 hour in ECM supplemented with 0.5% FBS, washed again. Treatments consisting of ECM only, 2.5% serum (lupus or control) in ECM or 100 ng/mL TNF-alpha (Thermo Fisher Scientific) (positive control) in ECM were added to the HRGECs, and cells underwent incubation for 3 hours at 37° C. HRGECs were then washed three times with RPMI-1640 (without phenol red); 6×10⁵ Calcein AM-labelled neutrophils were added to each well of the treated HRGECs. Cells were incubated for 20 min at 37° C. and washed five times with PBS (without Ca²⁺/Mg²⁺). Fluorescence intensity was quantified before the first wash and after the fifth wash to determine percent cell adherence, with all values then normalized to untreated controls. Fluorescence intensity was measured from the top of the plate at 485 nm excitation, 520 nm emission using a fluorescence microplate reader (Biotek HT Synergy).

### Statistical Analysis

All samples were assayed in triplicate, and statistical analysis was performed using GraphPad Prism Software (V.8.4) or SPSS V.24.0. Depending on the distribution of the data, unpaired t-test, paired t-test or one-way analysis of variance with Tukey's multiple comparisons were used to compare groups, while Spearman's or Pearson's correlation testing was performed to determine associations between variables. A generalized estimating equation was used to determine the association between cytokines and migration, accounting for two repeated measures in the same subject (during active and inactive disease). Both within-subject variables (presence of disease activity) and independent variables (cytokine levels in the conditioned medium) were modelled with neutrophil migration as the response variable.

### Pathways Analysis

To identify possible pathways leading to and resulting from cytokine expression, highly correlated cytokines expressed by lupus-serum-stimulated endothelial cells were identified. Reactome pathways analysis was performed (Fabregat et al., Nucleic Acids Res 2016; 44:D481-7). Overrepresentation analysis was performed to determine over representation of pathways related to the selected cytokines. Probability scores were produced and corrected for false discovery rate (FDR) using the Benjamani-Hochberg method. Results were filtered for pathways described in HomoSapiens. Only pathways with FDR p values<0.05 were selected and described.

The results of these experiments are now described.

SLE Serum Promotes Neutrophil Chemotaxis to Glomerular Endothelial Cells, which is Enhanced in Patients with Hypertension, Lupus Nephritis and Disease Activity

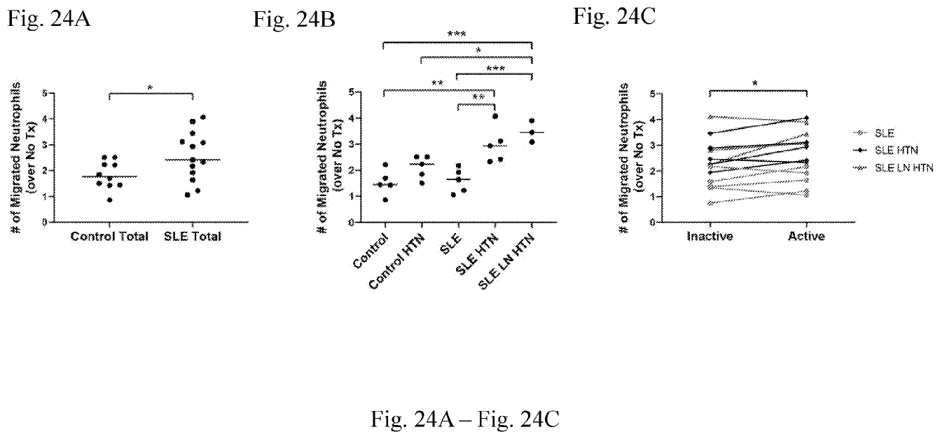

Chemotaxis of inflammatory cells towards an activated endothelium is necessary for inflammation in affected tissues. In order to determine whether serum from patients with SLE impacts endothelial function, the ability of serum-treated HRGECs to induce chemotaxis of neutrophils was assessed. In these experiments, HRGECs were treated with serum, washed and serum starved, then CM from subsequent culture was collected for use in the neutrophil chemotaxis assays. CM from HRGECs treated with lupus serum induced greater neutrophil migration over that of treatment with control serum (FIG. 24A). When examining specific clinical phenotypes, CM from cells treated with serum from patients with SLE and HTN and SLE with LN and HTN promoted greater neutrophil chemotaxis than treatment with control serum or SLE without HTN serum (FIG. 24B). The effect of serum from paired inactive and active disease visits was then compared with determine whether SLE activity-related factors promote neutrophil chemotaxis. CM from HRGECs treated with serum from patients during active disease visits (mean=2.56, SD=0.96) induced greater chemotaxis than that from paired inactive disease visits (mean=2.26, SD=0.91) (t=2.46, df=12, p=0.03) (FIG. 24C).

Cytokines Secreted by SLE Serum-Exposed HRGECs Correlate with Neutrophil Chemotaxis

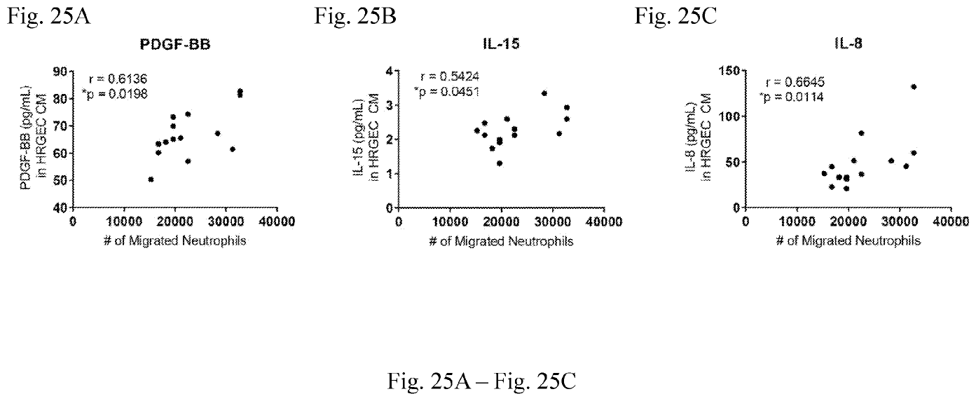

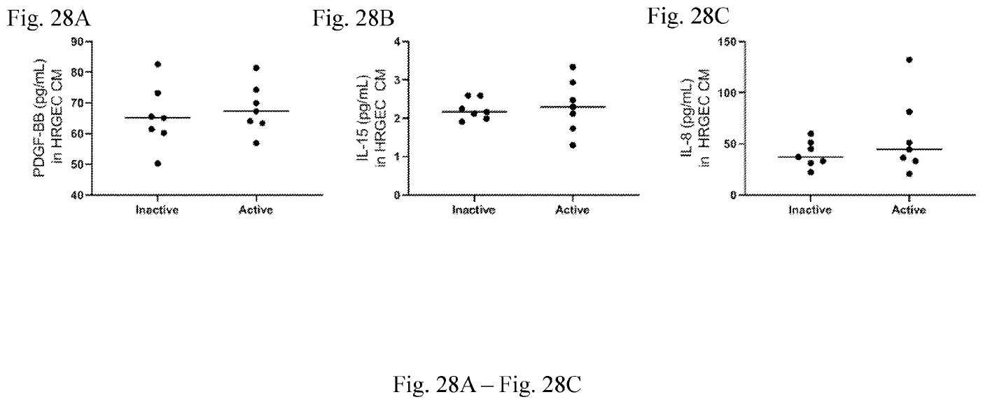

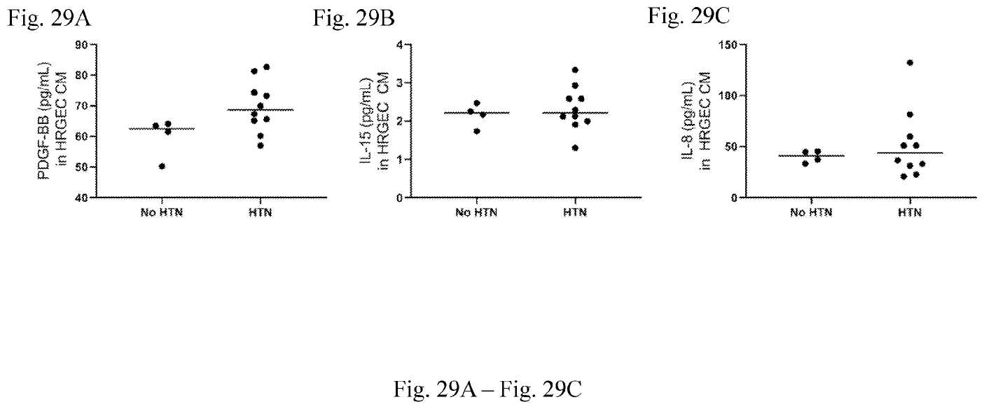

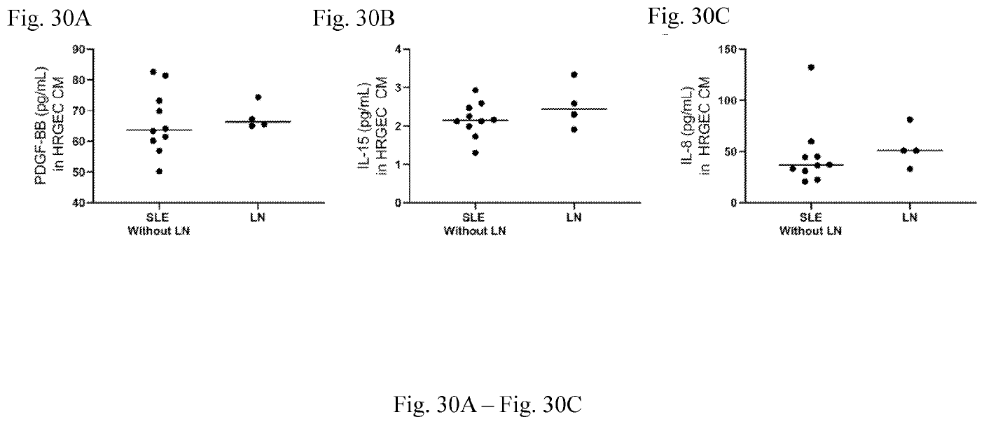

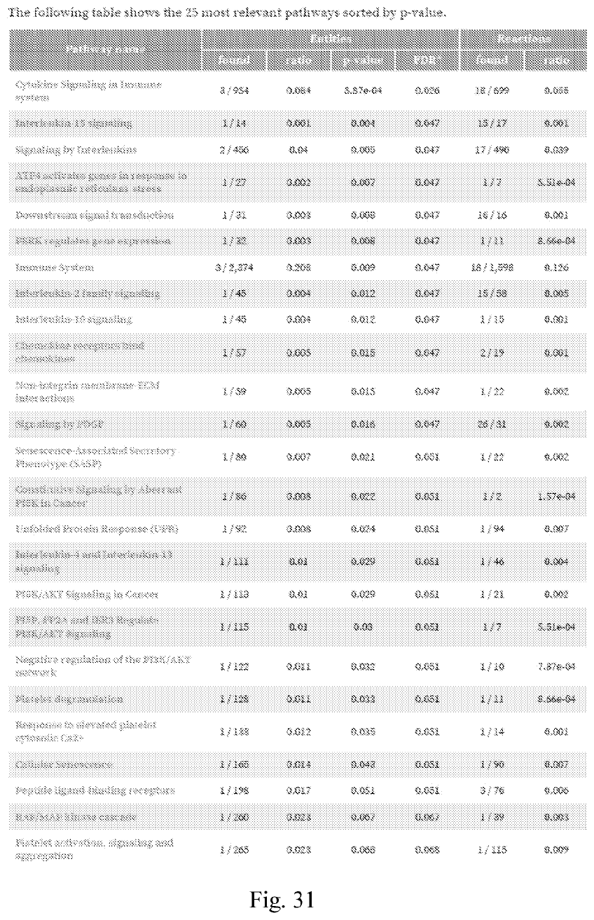

To explore which factors are released by serum-activated endothelial cells that may contribute to neutrophil migration, the concentration of candidate factors in the CM was analyzed by a multiplex array. The levels of those factors were correlated with the migration of neutrophils towards the same CM. Levels of platelet-derived growth factor-BB (PDGF-BB) (FIG. 25A), IL-15 (FIG. 25B) and IL-8 (FIG. 25C) all positively correlated with neutrophil migration. In FIG. 31, it was shown that disease activity, HTN and renal involvement led to differences in neutrophil chemotaxis; however, differences between these groups in the cytokine profiles were not found (FIG. 28-FIG. 30).

### Pathways Common to Highly Correlated Induced Factors are Important in Oxidative Stress and Endoplasmic Reticulum Stress

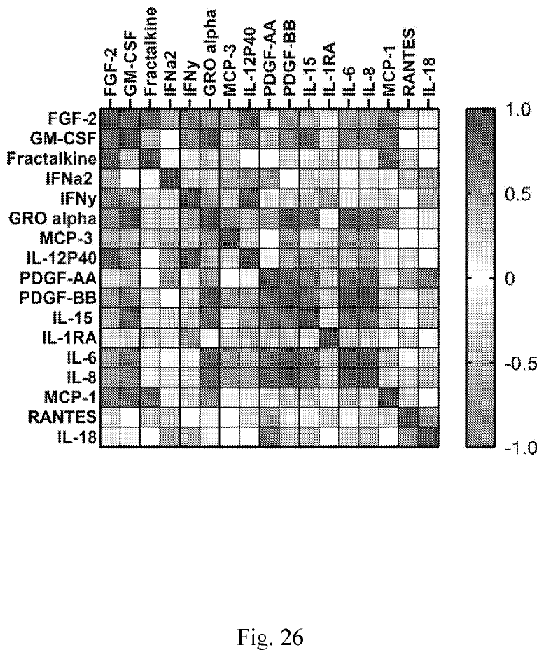

To suggest common pathways induced by SLE serum during disease activity, it was determined which secreted factors in the CM from serum-treated endothelial cells were highly correlated with each other (FIG. 26). PDGF-BB positively correlated with IL-8 and IL-6, with IL-8 and IL-6 positively correlating with each other. Growth-regulated oncogene alpha (GRO-alpha or CXCL1) positively correlated with granulocyte macrophage colony stimulating factor, PDGF-BB and IL-6 (Table 9). To understand further the functional relationships of some of these factors, the Reactome Knowledgebase was used to ascertain which pathways related to IL-8, PDGF-BB and IL-15 may be involved in neutrophil migration (Jassal et al., Nucleic Acids Res 2020; 48:D498-503). The most significant pathways were intuitive and included: immune system, cytokine signalling, IL-2 family signalling (including IL-15 signalling), signalling by PDGF, non-integration membrane-ECM interactions, chemokine receptors and downstream signal transduction. However, a few were of interest that were less intuitive, including: activating transcription factor 4 (ATF4), protein kinase R-like endoplasmic reticulum kinase (PERK) regulation of gene expression and IL-10 signalling (see FIG. 31).

TABLE 9
Correlation between cytokines expressed
by HRGECs stimulated with lupus serum

| Cytokine 1 | Cytokine 2 | r value | P value |
|---|---|---|---|
| PDGF-BB | IL-8 | 0.96 | 4.91E−05 |
| PDGF-BB | IL-6 | 0.95 | 1.08E−04 |
| IL-8 | IL-6 | 0.89 | 0.001 |
| GRO-alpha | GM-CSF | 0.86 | 0.002 |
| GRO-alpha | PDGF-BB | 0.86 | 0.003 |
| GRO-alpha | IL-6 | 0.83 | 0.005 |

### SLE Serum Promotes Neutrophil Adhesion to HRGECs

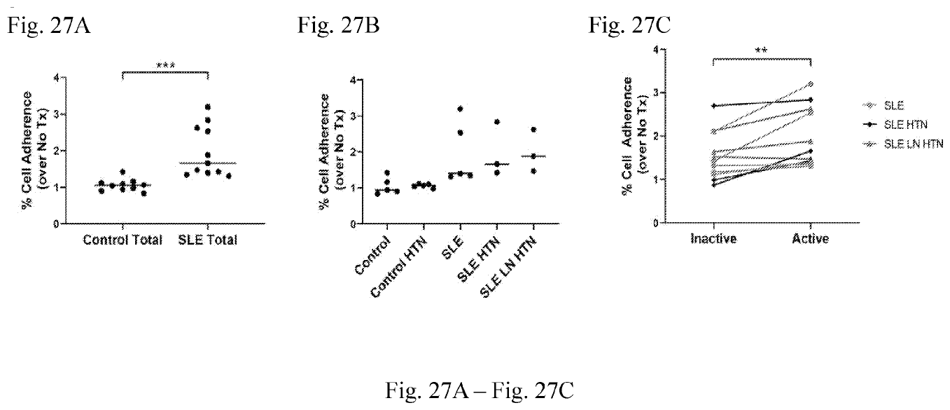

One of the hallmarks of endothelial dysfunction is adhesion of circulating neutrophils to the endothelium. The impact of lupus serum on endothelial dysfunction was determined by examining neutrophil adhesion to washed HRGECs after treatment with either control or lupus serum. HRGECs treated with lupus serum increased neutrophil adhesion over that of control serum (FIG. 27A). While trends were present, no significant difference was seen between control, control HTN, SLE, SLE HTN and SLE LN HTN serum treatment (FIG. 27B). Endothelial cells treated with serum from patients during active disease visits (mean=1.97, SD=0.69) induced greater neutrophil adhesion than those treated with serum from the same patients during paired visits with inactive disease (mean=1.543, SD=0.56) (t=3.48, df=10, p=0.006) (FIG. 27C).

### SLE Serum Factors Induce an Inflammatory Response

This study demonstrated that factors in SLE serum can induce an inflammatory response that enhances neutrophil migration and adhesion in human glomerular endothelial cells. Neutrophil migration was shown to increase with SLE HTN and LN HTN compared with control, and also increased with disease activity, while neutrophil adhesion increased with disease activity. Chemokine production (IL-8, IL-15 and PDGF-BB) by SLE and LN serum-stimulated HRGECs associated with migration of neutrophils to the same medium. Taken together, these findings suggest that glomerular endothelial cells play an active role in the inflammatory response in lupus and LN, and that unique factors in the serum induce this response.

A heterogeneity of factors in SLE serum may lead to this response. Several reports suggest receptor-mediated and redox signalling mechanisms lead to endothelial dysfunction (Mai et al., J Hematol Oncol 2013; 6:61; Sumiyoshi et al., Cardiovasc Res 2008; 80:463-70; Jones Buie et al., Am J Med Sci 2014; 348:168-75; Stark et al., Faseb J 2018; 32:945-56; Urbanski et al., PLoS One 2018; 13:e0206814; Dorosz et al., Inflamm 2015; 2015:1-16; Atehortúa et al., Arthritis Res Ther 2019; 21:34). Further, it has been published that eNOS is uncoupled with addition of lupus serum (Jones Buie et al., Lupus Sci Med 2019; 6:e000294). This uncoupling leads to production of reactive oxygen species in an animal model of LN and in LN glomerular tissue (Gilkeson et al., PLoS One 2013; 8:e64650; Oates et al., Lupus 2013; 22:1361-70). Therefore, reduction oxidation (redox) signalling may be a common intracellular mechanism in endothelial cell activation. Factors contained in microparticles and immune complexes also lead to endothelial cell activation (Atehortúa et al., Arthritis Res Ther 2019; 21:34). Isolated chemokines known to be associated with LN can, in combination, induce expression of inflammatory cytokines and growth factors in immortalized glomerular endothelial cells (Dimou et al., Sci Rep 2019; 9:8348).

HTN was associated with a serum-induced chemotaxis of neutrophils to HRGECs. HTN itself leads to endothelial dysfunction. Angiotensin signalling via the angiotensin II receptor induces NADPH oxidase-dependent reactive intermediate production in endothelial cells and reduces the modulating effect of eNOS (Nickenig et al., Circulation 2002; 105:393-6) by an uncoupling of the eNOS homodimer (Landmesser et al., J Clin Invest 2003; 111:1201-9). These combined findings support the hypothesis that endothelial dysfunction induced by HTN and lupus are either synergistic or share common mechanisms.

This study suggests several mechanisms for serum-induced neutrophil migration that are biologically plausible in SLE. PDGF-BB is chemotactic for neutrophils and monocytes and may act in to induce vascular injury in LN (Cai et al., Kidney Blood Press Res 2019; 44:643-55). While the literature on expression of functional PDGF receptors on neutrophils is conflicting (Qu et al., J Immunol 1995; 154:4133-41), endothelial cells express PDGFRB, which signals through phosphatidylinositol 3-kinase activation (Wang et al., Cancer Res 1999; 59:1464-72). In turn, PDGF-BB can act in an autocrine fashion to stimulate endothelial production of CCL2/monocyte chemotactic protein-1 (MCP-1) (Au et al., Am J Pathol 2009; 175:294-302). IL-8, on the other hand, is chemotactic to neutrophils through neutrophil expression of CCR1 and CCR2 (Miyabe et al., Nat Rev Rheumatol 2019; 15:731-46). IL-8 also induces neutrophil oxidative burst and extracellular traps formation (Mao et al., Biomark Med 2018; 12:1291-302). Urine levels of this factor are associated with lack of response to therapy in human LN (Wolf et al., Arthritis Rheumatol 2016; 68:1955-63), and a polymorphism of IL-8 is associated with poor outcome in African-Americans (Rovin et al., Kidney Int 2002; 62:261-5). IL-15 acts as a chemokine to several cell types including neutrophils, which express the IL-15Rα chain (Girard et al., Clin Immunol Immunopathol 1998; 88:232-40). Levels of IL-15 are elevated in humans with lupus (Aringer et al., Rheumatology 2001; 40:876-81).

This study suggests that LN serum may activate pathways known to be involved in oxidative stress and endoplasmic reticulum (ER) stress. During ER redox imbalance, ER stress can induce dissociation of HSP90 from PERK, which decreases HSP90 stability and causes the dissociation of eNOS from HSP90, leading to eNOS uncoupling (Lee et al., Free Radic Biol Med 2017; 113:26-35). Furthermore, PERK can phosphorylate and activate ATF4 to induce expression of anti-oxidative genes (Cullinan et al., J Biol Chem 2004; 279:20108-17; Cullinan et al., Mol Cell Biol 2003; 23:7198-209; Dey et al., J Clin Invest 2015; 125:2592-608). ATF4 is activated in ER stress in endothelial cells and in turn induces expression of chemokines such as IL-8 and MCP-1 (Gargalovic et al., Proc Natl Acad Sci USA 2006; 103:12741-6; Gargalovic et al., Arterioscler Thromb Vasc Biol 2006; 26:2490-6) in response to oxidized lipids. Thus, the PERK/ATF4 axis plays an important role in cellular stress conditions, including oxidative stress which greatly impacts the functionality of the endothelium. Lastly, IL-10 is known to inhibit nitric oxide production, inhibit endothelial cell differentiation and repair and may enhance the negative effect of interferon-alpha on endothelial function (Cates et al., Rheumatology 2015; 54:1114-23).

Of note, low-density lipoprotein (LDL) was higher in some patients with active disease. While these differences did not achieve statistical significance (p=0.37 for total cholesterol and p=0.32 for LDL), they are consistent with reports by Durcan et al in which LDL levels correlated with disease activity (Durcan et al., J Rheumatol 2016; 43:745-50). Because oxidized LDL was not measured, it cannot be determined if LDL may have induced endothelial dysfunction through oxidized LDL receptors.

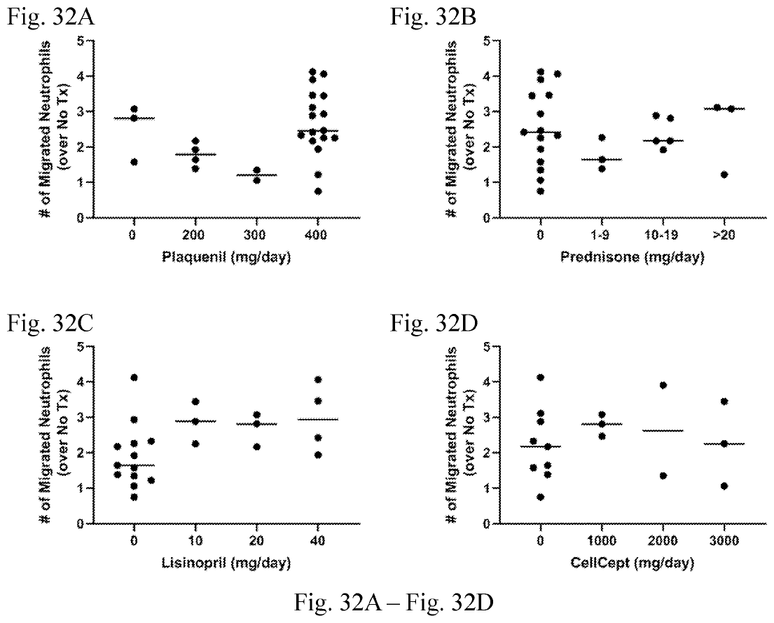

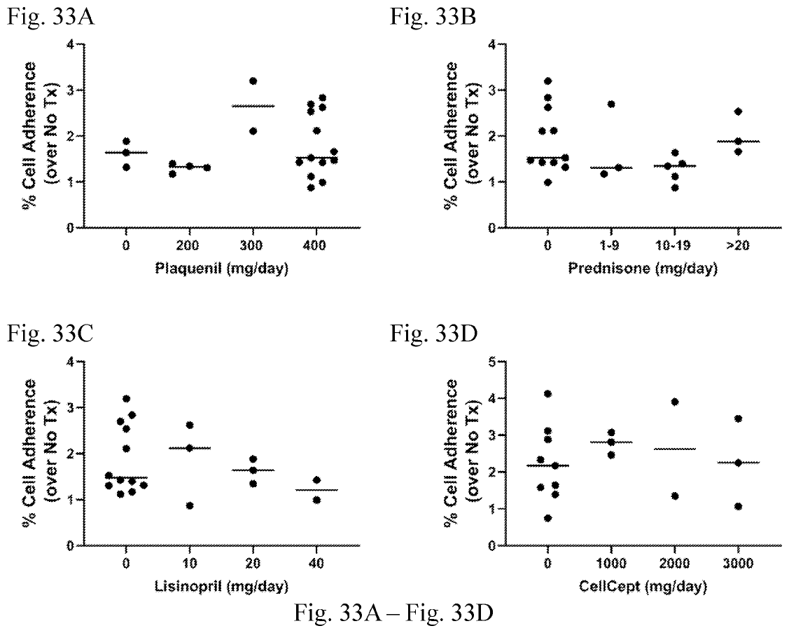

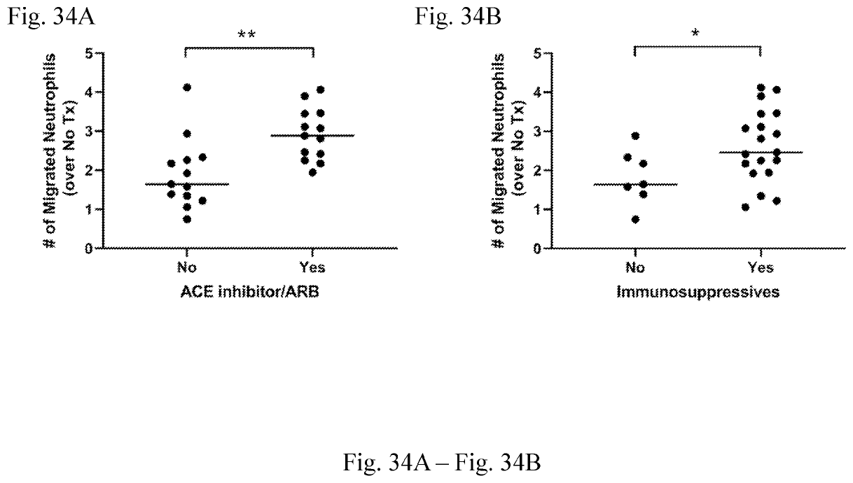

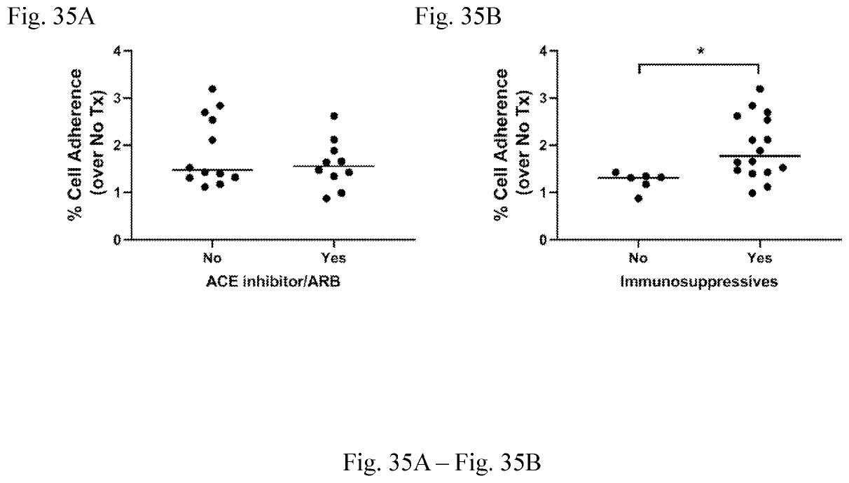

Patients in this study were prescribed a variety of medications (Table 7). No difference in either neutrophil migration or adhesion was found when examining dosing effects of Plaquenil, Prednisone, Lisinopril or CellCept (FIG. 32 and FIG. 33). However, when examining use of immunomodulatory medications (as a group) or ACE inhibitors/angiotensin receptor blockers, increased neutrophil migration with use of both medication groups and increased neutrophil adhesion for immunomodulatory medications was found (FIG. 34 and FIG. 35) This is the opposite effect of what was anticipated and may indicate a bias from indication. It also suggests factors in serum that are important for adhesion and migration of neutrophils to the endothelium may not be controlled by these medications. Further, while this study focused on SLE, diabetic and hypertensive renal disease are also known to be associated with endothelial dysfunction that improves with therapy targeting eNOS dysfunction (Pannirselvam et al., Br J Pharmacol 2003; 140:701-6; Keller et al., Oxid Med Cell Longev 2018; 2018:1-10; Thida et al., Am J Hypertens 2010; 23:569-74).

This study established that factors in the serum of patients with lupus and LN induce an endothelial cell inflammatory phenotype in renal glomerular endothelial cells, which is manifested by neutrophil adhesion and migration. It also demonstrates the effect of disease activity on this phenotype using paired visit serum samples from individual patients during inactive and active disease states. This study demonstrates associations between migration of neutrophils and serum-induced chemokine production in glomerular endothelial cells. Common pathways associated with these factors are important in ER stress, oxidative stress and uncoupling of eNOS. The findings from this study reinforce the importance of glomerular endothelial cells in trafficking neutrophils to sites of inflammation through generation of pathogenic chemotactic factors. They further provide the rationale for studying redox-induced pathways and ER stress as mechanisms for glomerular inflammation in LN.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition for treating or preventing a chronic inflammatory autoimmune disease or disorder comprising a stimulator of nitric oxide (NO) production.

2. The composition of claim 1, wherein the stimulator of NO production is a small molecule chemical compound.

3. The composition of claim 2, wherein the small molecule chemical compound is selected from the group consisting of 2-amino-6-[-2-hydroxypropanoyl]-7,8-dihydro-1H-pteridin-4-one (L-Sepiapterin), tetrahydrobiopterin (BH4), nitroglycerin (GTN), isosorbide dinitrate (ISDN), isosorbide mononitrate (IS-5N), amyl nitrite, sodium nitroprusside (SNP), pentaerythrityltetranitrate (PETN), s-nitrosoglutathione, nicorandil and molsidomine, and salts thereof.

4. The composition of claim 1, wherein the composition further comprises an immunosuppressive agent.

5. The composition of claim 1, wherein the disease or disorder is selected from the group consisting of systemic lupus erythematosus (SLE), lupus nephritis, autoimmunity-related cardiovascular disease (CVD) and atherosclerosis, hypertension, systemic sclerosis, celiac disease, inflammatory bowel disease, type 1 diabetes, glomerulonephritis, Crohn's disease, psoriatic arthritis, and rheumatoid arthritis.

6. A method for treating or preventing a chronic inflammatory autoimmune disease or disorder in a subject in need thereof, the method comprising administrating to the subject a composition comprising a stimulator of nitric oxide (NO) production.

7. The method of claim 6, wherein the stimulator of NO production is a small molecule chemical compound.

8. The method of claim 6, wherein the small molecule chemical compound is selected from the group consisting of 2-amino-6-[-2-hydroxypropanoyl]-7,8-dihydro-1H-pteridin-4-one (L-Sepiapterin), tetrahydrobiopterin (BH4), nitroglycerin (GTN), isosorbide dinitrate (ISDN), isosorbide mononitrate (IS-5N), amyl nitrite, sodium nitroprusside (SNP), pentaerythrityltetranitrate (PETN), s-nitrosoglutathione, nicorandil and molsidomine, derivatives thereof, and salts thereof.

9. The method of claim 6, further comprises administering an immunosuppressive agent.

10. The method of claim 6, wherein the disease or disorder is selected from the group consisting of SLE, lupus nephritis, autoimmunity-related CVD and atherosclerosis, hypertension, systemic sclerosis, celiac disease, inflammatory bowel disease, type 1 diabetes, glomerulonephritis, Crohn's disease, psoriatic arthritis, and rheumatoid arthritis.

11. The method of claim 6, wherein the disease or disorder is active.

12. The method of claim 6, wherein the method comprises administering the stimulator of NO production to a subject having inactive disease, whereby the method prevents the disease from becoming active.

13. The method of claim 6, wherein the stimulator of NO production is L-Sepiapterin.

14. The method of claim 13, wherein the L-Sepiapterin is administered at a dosage of between 5 and 50 mg/kg daily.

15. The method of claim 14, wherein the L-Sepiapterin is administered at a dosage of 20 mg/kg daily.

16. The method of claim 6, wherein the subject is a human.
